(19) United States
(12) Patent Application Publication
(10) Pub. No.: US 2024/0024439 A1
(43) Pub. Date: Jan. 25, 2024

(54) ADMINISTRATION OF ANTI-TUMOR VACCINES

(71) Applicants: IOGENETICS, LLC, Madison, WI (US); ASTHRA, LLC, Pacific Palisades, CA (US)

(72) Inventors: Jane Homan, Marshall, WI (US); Robert D. Bremel, Marshall, WI (US); Santosh Kesari, Santa Monica, CA (US)

(21) Appl. No.: 18/256,242

(22) PCT Filed: Dec. 7, 2021
(86) PCT No.: PCT/US2021/062147

§ 371 Date: Jun. 7, 2023

Related U.S. Application Data
(60) Provisional application No. 63/122,192, filed on Dec. 7, 2020.

(51) Int. Cl. A61K 39/00; A61P 35/00; G01N 33/574; C12Q 1/6886

(52) U.S. Cl. CPC A61K 39/0011; A61P 35/00; G01N 33/57484; C12Q 1/6886; A61K 2039/575; G01N 2333/70539

(57) ABSTRACT

The present invention relates to methods for stimulating the immune response to a tumor by implementing a two-phase vaccination strategy in which a first vaccination comprises epitopes from tumor associated antigens or epitopes which embody amino acid mutations commonly associated with the cancer that is administered before tissue diagnosis, and a second vaccination which comprises personal neoepitopes, the design of which is unique to that subject and is identified based on comparative sequencing of normal tissue and tumor tissue obtained by biopsy.

### FIELD OF THE INVENTION

The present invention relates to methods for stimulating the immune response to a tumor by implementing a two-phase vaccination strategy in which a first vaccination comprises epitopes from tumor associated antigens or epitopes which embody amino acid mutations commonly associated with the cancer that is administered before tissue diagnosis, and a second vaccination which comprises personal neoepitopes, the design of which is unique to that subject and is identified based on comparative sequencing of normal tissue and tumor tissue obtained by biopsy.

### BACKGROUND OF THE INVENTION

Cancer arises as the combination of an array of genomic, proteomic and immunologic events which make every cancer a unique challenge. Mutational events at the genomic level are manifest in changed protein functions that give rise to the classic features of cancer: dysregulation comprising cell proliferation, evasion of growth suppressors, invasiveness and metastasis, ongoing cell replication and resistance to apoptosis, increased vascularity and dysregulation of cell energetics [1]. However, tumors also exhibit a further array of consequences of mutations in their evasion of the immune response which could otherwise limit progression. The present invention addresses how to marshal the immune response to directly limit invasiveness by limiting the actions of some tumor associated proteins, to limit progression from initial diagnosis until more definite treatments weeks-months later in current standard paradigms, and to reduce the immune evasion but stimulating tumor specific cytotoxic T cells that can bring about the elimination of cancer cells. A goal of the present invention is to combine early stimulation of the immune response to cancer with the stimulation of a tumor specific response. In the first instance early stimulation is achieved by a combination of epitopes in tumor associated antigens and/or in proteins with mutations which are very commonly found in association with a particular clinical presentation. In the second instance a more tumor specific response is achieved by recognizing that the mutational landscape in each tumor is unique and personal to that patient. This is the result of the overlay of the mutations which have occurred and continue to occur as the tumor progresses, and the immunosurveillance which is determined by the MHC binding characteristics of the peptides comprising the mutations, the MHC alleles carried by the patient, and the particular repertoire of T cells that the individual patient carries. This results in a unique array of neoepitopes which characterize the immunomic profile of a given tumor and which serve as tumor specific targets.

As tumorigenesis progresses within a given patient the array of mutations diversifies to adopt epitope patterns unique to that individual. Targeting the immune response to such unique neoepitopes requires identification of the mutations that differentiate tumor cells form normal tissue and selection or design of peptides, or their encoding nucleic acids, which embody the tumor specific mutation and can present it to stimulate the T cells of that individual.

### SUMMARY OF THE INVENTION

The present invention provides a two-phase immunotherapeutic intervention strategy for treatment of a subject with a clinical diagnosis of cancer. In one embodiment the strategy comprises a first intervention, comprising administration of a first round of vaccination in which the antigens administered to the subject are peptides, or the nucleic acids which encode them, derived from one or both of two groups of proteins. The first group of proteins are tumor associated antigens which are not mutated, but which are known to be commonly upregulated in the type of cancer with which the subject is diagnosed, or to exert an influence favoring the progression of that type of tumor. The second group of proteins from which peptides administered in the first round of vaccination may be drawn are the most commonly mutated proteins associated with the type of cancer with which the subject is diagnosed.

Peptides selected from the second group encompass the common mutations found in the proteins. Following the first round of vaccination, the second intervention comprised in the invention is a second round of vaccination. The second round of vaccination is a personalized vaccination comprising a set of peptides, or the nucleic acids which encode them, that embody the amino acids shown to be mutated in that tumor by means of comparative sequencing of the proteins in the tumor and in normal tissue. The amino acids shown to be mutated are placed within the T cell exposed positions. The amino acids in the MHC groove exposed position, which are those positions not exposed to the T cells, are then substituted with alternative amino acids to achieve a desired binding affinity to the subject's MHC alleles. Thus, an array of alternative peptides is selected which is administered as a personalized vaccination, either as peptides or the nucleic acids that encode them.

Peptides selected from the proteins known to be commonly mutated are selected for the first vaccination so as to place the commonly mutated amino acids in one of the T cell exposed positions when the protein is bound in a MHC molecular groove. The amino acids in the MHC groove exposed position of these peptides, which are those positions not exposed to the T cells, may then substituted with alternative amino acids to achieve a desired binding affinity to the subject's MHC alleles.

In a further embodiment, the alternative peptides included in the personalized vaccination are selected from those mutated proteins which are shown to be expressed in the tumor, as evidenced by the ratio of copy numbers of the gene product detected in RNA relative to the copies of that gene detected in DNA. In preferred embodiments the gene product is detected in at least 10% of the RNA transcribed from that gene locus. It is also preferred that the alternative peptides are selected from mutants that are detected in at least 10% of the tumor DNA.

In a preferred embodiment, the first round of vaccination is administered to the subject prior to any surgical intervention. In a further preferred embodiment, the HLA alleles of the subject are determined as an aid to the selection of peptides included in the first round of vaccination. In the second round of vaccination a preferred embodiment includes five or more different peptides based on the demonstrated mutations found in the tumor proteins.

The present invention includes the design of immunogens which target either CD8+ T cells responsive to peptides bound in MHC I alleles or CD4+ T cells responsive to peptides bound in MHC II alleles. In the first of these instances, for targeting CD8+ T cells, the preferred embodiment is that the peptide is 8-10 amino acids in length, whereas in the second instance peptides of 13-20 amino acids are the preferred embodiment to target CD4+ T cells.

In yet other embodiments peptides of 8-35 amino acids may be deployed to target both CD8+ and CD4+ T cells following endopeptidase processing by antigen presenting cells.

The peptides, or their encoding nucleic acids, are designed and selected to be competitive with other peptides from the same protein and to exceed the binding affinity of 85% of the peptides in the originating proteins. In yet other embodiments the peptides are selected to exceed the binding affinity of 95% of peptides in the source protein. In preferred embodiments the peptides selected have a binding affinity to the MHC alleles of the subject of less than 200 nanomolar, in yet further embodiments a higher binding affinity of less than 50 nanomolar is provided.

While the present invention provides a method to design the second round of vaccination as a personalized vaccine by inclusion of alternative amino acids to optimize the binding affinity to MHC, in some embodiments it may also comprise natural peptides that occur in the tumor proteins where the binding affinity of the peptides is appropriate.

In some instances, each round of vaccination may comprise only one application of each group of peptides, In preferred embodiments each round of vaccination comprises multiple applications of the peptides or their encoding nucleic acids.

The present invention pertains to a strategy for immunotherapeutic intervention in a tumor drawn from the group of solid tumors including, but not limited to, those affecting tissues of the nervous system, lung, breast, pancreas, liver, skin, prostate, genito-urinary tract, hematopoietic system, gastro-intestinal, endocrine system, musculoskeletal or any other tissue affected by a tumor. In some preferred embodiments the methods provided by the invention are applied to the treatment of brain tumors, including but not limited to glioma, glioblastoma, neuroblastoma, meningioma, schwannoma, and metastases to the brain from other sites in the body. The first round of vaccination is selected according to the clinical diagnosis. In the case of a brain tumor, the peptides included the first round of vaccination may be drawn from the group comprising vascular endothelial growth factor receptors, vimentin, Wilms tumor protein or any other tumor associated protein common to that type of brain tumor. These examples are illustrative and not considered limiting. The invention provides sequences of peptides from vascular endothelial growth factor receptors, vimentin and Wilms tumor protein which are examples of suitable vaccinal peptides from these proteins.

The invention further provides that the first round of vaccination may comprise peptides, or their encoding nucleic acids, that comprise the most common mutations characteristic of that clinically diagnosed tumor. To that end the present invention provides sequences for peptides that embody the T cell exposed motifs of the most common mutations of epidermal growth factor receptor (EGFR), a common deleterious splice variant thereof, EGFRvIII, isocitrate dehydrogenase (IDH) mutant R132H, and histone variants H3.3 associated with gene instability mutant K27M. In yet other embodiments, peptide sequences are provided that encompass the most common mutation of the serine/threonine-protein kinase B-raf (BRAF) V600E. For each the invention identifies the T cell exposed motifs of interest and provides examples of peptides which are designed to bind optimally to exemplar MHC alleles. As many different alternative peptides can be designed to achieve the desired MHC binding affinity while maintaining the T cell exposed motif constant, the peptides provided are considered as non-limiting illustrations of the methods.

In some embodiments, the tumor associated protein is a viral protein, where such virus is an oncogenic virus such as a papillomavirus, retrovirus, polyomavirus or herpesvirus. In other instances the viral protein maybe drawn from the group of viruses which may be upregulated in the tumor cells and thus provide a target to direct a T cell response to that tumor cell. In particular embodiments such viruses include, but are not limited to, human papillomaviruses and human cytomegalovirus.

In addition to providing a method that is a strategy for immunotherapeutic intervention, the present invention also provides particular synthetic peptides with diverse HLA binding that serve as immunogens to direct a T cell response to VEGFR1 and VEGFR2, vimentin and WT1. Of particular note here is that these peptides are selected based on their high binding affinity to a multiplicity of MHC alleles, facilitating their use in subjects which have not been HLA typed. Such peptides are provided to bind to MHC I and MHC II alleles and with a binding affinity of exceeding 50 nanomolar and exceeding 200 nanomolar. In particular embodiments the peptides derived from VEGFR1 and VEGFR2, vimentin and WT1 are shorter, being of 8-10 amino acids, whereas in yet other embodiments they are of 13-20 amino acids and in yet other instances are up to 30 amino acids in length. As is well known to those skilled in the art, the peptides may also be delivered by means of the nucleic acids which encode them. Many routes of administration are suitable. In some embodiments the peptides are administered parenterally, in some instances by intradermal or subcutaneous delivery. In yet other embodiments administration of one or more doses of vaccine is orally. The vaccine composition may comprise an adjuvant. Various delivery vehicles may be utilized such that in one embodiment the peptides or nucleic acids encoding them may be delivered by a lipid drug delivery system drawn from the group comprising, but not limited to, lipid nanoparticles, emulsions, self-emulsifying drug delivery systems, nanocapsules and liposomes or by a viral vector, which may comprise, but is not limited to, elements of a retroviral vector, a poxvirus, an adenovirus, or other virus vector delivery system. In yet other embodiments in lieu of administering the vaccine directly to the subject, antigen presenting cells are harvested for the subject and these are contacted with the vaccinal peptides of interest in vitro, prior to returning these to the subject, or exposing the antigen presenting cells to T cells also drawn from the subject, before autologous reinfusion.

It is anticipated that in some preferred embodiments the immunotherapeutic strategy described in the present invention will be accompanied by other immunotherapeutic interventions such as but not limited to the treatment with check point inhibitors.

A further embodiment of the present invention addresses the monitoring of the immune response the subject mounts to any one of the epitopes embodied in the first or second round of vaccination. Thus, a further application of the present invention is in companion diagnostics in which the peptides identified herein are embodied into a diagnostic test.

In some further preferred embodiments, the present invention provides methods of treatment of a subject clinically presenting with a tumor comprising: administering a first round of vaccination, wherein the vaccination comprises an array of one or more peptides, or nucleic acids encoding the peptides, selected from the group consisting of non-mutated peptides derived from tumor associated antigens appropriate to tumors of the type diagnosed and peptides commonly found to be mutated in tumors of the type diagnosed; and administering a second round of personalized vaccination, wherein the second round of vaccination comprises the following steps: obtaining a biopsy of the tumor and of normal tissue from the subject, and obtaining sequences for DNA, RNA and proteins in the biopsy; identifying proteins from the biopsy containing mutated amino acids and the peptide comprising each of the mutated amino acids; determining T cell exposed motifs which comprise mutated amino acids in each of the proteins; determining the predicted binding affinity to the subject's MHC alleles of peptides which comprise each of the T cell exposed motifs, or a subset thereof; generating an array of alternative peptides not present in the tumor, wherein each peptide in the array comprises the amino acids of one of the T cell exposed motifs, and in which the amino acids not within the T cell exposed motif are substituted to change the predicted MHC binding affinity; selecting a group of one or more selected peptides from the array of alternative peptides which have a desired predicted binding affinity for one or more of the subject's MHC alleles; and synthesizing the group of one or more selected peptides, or nucleic acids encoding the selected peptides and administering the selected peptides, or their encoding nucleic acids, as the second round of vaccination.

In some preferred embodiments, the methods further comprise selecting the peptides commonly found to be mutated in tumors so as to position the mutated amino acid in a T cell exposed position and substituting one or more of the amino acids not in a T cell exposed position to provide a desired binding affinity to one or more of the MHC alleles of the subject.

In some preferred embodiments, the methods further comprise: a) determining the fraction of the DNA in the tumor biopsy which encodes each of the mutated amino acids and the fraction of RNA transcribed from that gene locus and expressing the mutated amino acids; and b) selecting a sub array of the alternative peptides from the proteins in the biopsy which are present in at least 10% of the DNA in the biopsy and expressed in at least 10% of the RNA transcribed from that gene locus in the biopsy.

In some preferred embodiments, the method further comprise determining the MHC alleles of the subject prior to the first round of vaccination.

In some preferred embodiments, the first round of vaccination is administered prior to surgical intervention.

In some preferred embodiments, the MHC alleles are MHC type I alleles and the T cell response is a CD8+ response. In some preferred embodiments, the MHC alleles are MHC type II alleles and the T cell response is a CD4+ response. In some preferred embodiments, the MHC alleles are a combination of MHC type I alleles and MHC type II alleles. In some preferred embodiments, the MHC I allele in the first round of vaccination is not A0201 or A2402.

In some preferred embodiments, the peptides, or nucleic acids encoding them, administered in the first round or the second round are 8 to 10 amino acids long. In some preferred embodiments, the peptides, or nucleic acids encoding them, administered in the first round or the second round are 13-20 amino acids long. In some preferred embodiments, the peptides, or nucleic acids encoding them, administered in the first round or the second round are 8-35 amino acids long.

In some preferred embodiments, the group of one or more selected peptides, or the nucleic acids encoding them, administered in the second round of vaccination comprises at least 5 unique peptides not present in the proteins sequenced in the tumor.

In some preferred embodiments, the desired predicted binding affinity to the one or more of the subject's MHC alleles of selected peptides in the second round of vaccination exceeds 85% of the binding affinity of all peptides in the tumor protein that comprises the mutated amino acid. In some preferred embodiments, the desired predicted binding affinity to the one or more of the subject's MHC alleles of selected peptides in the second round exceeds 95% of the binding affinity of all peptides in the tumor protein that comprises the mutated amino acid. In some preferred embodiments, the desired predicted binding affinity to the one or more of the subject's MHC alleles of selected peptides in the second round is less than 50 nanomolar. In some preferred embodiments, the desired predicted binding affinity to the one or more of the subject's MHC alleles of selected peptides in the second round is less than 200 nanomolar.

In some preferred embodiments, the first and/or second rounds of vaccination further comprise administering peptides, or the nucleic acids encoding them, which occur naturally in a tumor protein.

In some preferred embodiments, the first and second rounds of vaccination each comprise more than a single application of the array of peptides, or the nucleic acids encoding them. In some preferred embodiments, each round of vaccination comprises 3 or more applications of the array of peptides, or the nucleic acids encoding them.

In some preferred embodiments, the tumor is a tumor of a tissue selected from the group consisting of nervous system, lung, breast, pancreas, liver, skin, prostate, genito-urinary tract, hematopoietic system, gastro-intestinal, endocrine system, and musculoskeletal tissues.

In some preferred embodiments, the tumor of the nervous system tissue is selected from the group consisting of glioma, glioblastoma, neuroblastoma, meningioma, schwannoma, and metastases to the brain from other sites.

In some preferred embodiments, the non-mutated peptides administered in the first round of vaccination are selected from the group consisting of VEGFR, WT-1, and vimentin. In some preferred embodiments, the first round of vaccination utilizes peptides that are selected from the group consisting of SEQ ID NOs: 1-118 and combinations thereof.

In some preferred embodiments, the first round of vaccination further includes mutated peptides that are selected from the group consisting of EGFR, H3.3, and IDH. In some preferred embodiments, the mutated peptides comprise T cell exposed motifs selected from the group consisting of SEQ ID NOs: 154-183, 284-288, 294-298, 344-348, 392-396, and 441-446 and combinations thereof. In some preferred embodiments, the EGFR is the variant EGFRvIII. In some preferred embodiments, the mutated proteins from EGFR comprise the T cell exposed motifs selected from the group consisting of SEQ ID NOs: 284-288 and combinations thereof. In some preferred embodiments, the T cell exposed motifs are flanked by groove exposed motifs that do not occur naturally and in which amino acids have been substituted to enhance MHC binding.

In some preferred embodiments, the first round of vaccination further includes mutated peptides that are drawn from the protein group comprising KIAA1549-BRAF fusion and BRAF. In some preferred embodiments, the mutated peptides comprise the T cell exposed motifs selected from the group consisting of SEQ ID NOs: 468-472 and 512-514 and combinations thereof.

In some preferred embodiments, the tumor is a lung tumor. In some preferred embodiments, the non-mutated peptides administered in the first round of vaccination are selected from the group consisting of CEA, MAGE-A1, MAGE-A3, MAGE-A4, MSLN, PRAME, TERT, HER2, MUC1, BIRC5, STEAP1, SOX2, NY-ESO-1 and combinations thereof. In some preferred embodiments, the first round of vaccination further includes mutated peptides that are selected from the group consisting of KRAS, EGFR, PIK3CA, TP53, BRAF, EML4 ALK and combinations thereof.

In some preferred embodiments, the tumor of the skin is a melanoma. In some preferred embodiments, the non-mutated peptides administered in the first round of vaccination are selected from the group consisting of PMEL, MAGE family, MART, MAGEA3, NY-ESO-1 and combinations thereof. In some preferred embodiments, the first round of vaccination further includes mutated peptides that are selected from the group consisting of BRAF, NRAS, IDH, MAK2K1 and combinations thereof.

In some preferred embodiments, the tumor is a prostate tumor. In some preferred embodiments, the non-mutated peptides administered in the first round of vaccination are selected from the group consisting of PSMA, SSEA-4, MAGE-C2, PSA, NANOG and combinations thereof. In some preferred embodiments, the first round of vaccination further includes mutated peptides that are selected from the group consisting of SPOP, TP53, PIK3CA, IDH and combinations thereof.

In some preferred embodiments, the tumor is a breast tumor. In some preferred embodiments, the non-mutated peptides administered in the first round of vaccination are selected from the group consisting of MUC 1, CEA, ERBB2, MAGE, NY-ESO1, TACA and combinations thereof. In some preferred embodiments, the first round of vaccination further includes mutated peptides that are selected from the group consisting of ST6GALNAC6, MAGEA12, KDM5B, TEX15, ERBB2, ERBB3, CEA, MYC, TP53, MKI67, NME1, PRDX6, EIF5A, PARK7, HSPB1, PIK3CA, HSPA14 proteins and combinations thereof.

In some preferred embodiments, the tumor associated antigen is a viral protein. In some preferred embodiments, the viral protein is selected from the group consisting of herpesviruses and papillomaviruses.

In some preferred embodiments, the array of one or more peptides, or nucleic acids encoding the peptides administered in the first round comprises one or more synthetic peptides or nucleic acids encoding the peptides derived from VEGFR1 or VEGFR2 wherein the synthetic peptides bind one or more MHC alleles and the synthetic peptides are selected from the group consisting of SEQ ID NOs: 1-28 and combinations thereof. In some preferred embodiments, the array of one or more peptides, or nucleic acids encoding the peptides administered in the first round comprises one or more synthetic peptides or nucleic acids encoding the peptides derived from vimentin wherein the synthetic peptides bind one or more MHC alleles and the peptides are selected from the group consisting of SEQ ID NOs: 29-103 and combinations thereof. In some preferred embodiments, the array of one or more peptides, or nucleic acids encoding the peptides administered in the first round comprises one or more synthetic peptides or nucleic acids encoding the peptides derived from WT-1 wherein the synthetic peptides bind one or more MHC alleles and the peptides are selected from the group consisting of SEQ ID NOs: 104-118 and combinations thereof. In some preferred embodiments, the peptides bind one or more MHC I alleles. In some preferred embodiments, the peptides are 8-10 amino acids. In some preferred embodiments, the MHC I allele is not A0201 or A2402. In some preferred embodiments, the peptides bind one or more MHC II alleles. In some preferred embodiments, the peptides are 15-18 amino acids. In some preferred embodiments, the predicted binding affinity of the peptides to the one or more MHC is less than 20 nanomolar. In some preferred embodiments, the predicted binding affinity of the peptides to the one or more MHC is less than 50 nanomolar. In some preferred embodiments, the predicted binding affinity of the peptides to the one or more MHC is less than 200 nanomolar.

In some preferred embodiments, at least 2 peptides that bind to MHC I alleles or nucleic acids encoding the peptides that bind to MHC I alleles are selected for synthesis and/or coadministration to a subject in the second round of vaccination. In some preferred embodiments, at least 5 peptides that bind to MHC I alleles or nucleic acids encoding the peptides that bind to MHC I alleles are selected for synthesis and/or coadministration to a subject in the second round of vaccination. In some preferred embodiments, at least 10 peptides that bind to MHC I alleles or nucleic acids encoding the peptides that bind to MHC I alleles are selected for synthesis and/or coadministration to a subject in the second round of vaccination. In some preferred embodiments, at least 15 peptides that bind to MHC I alleles or nucleic acids encoding the peptides that bind to MHC I alleles are selected for synthesis and/or coadministration to a subject in the second round of vaccination. In some preferred embodiments, at least 20 peptides that bind to MHC I alleles or nucleic acids encoding the peptides that bind to MHC I alleles are selected for synthesis and/or coadministration to a subject in the second round of vaccination. In some preferred embodiments, the allele is not A0201 or A2402. In some preferred embodiments, at least 2 peptides that bind to MHC II alleles or nucleic acids encoding the peptides that bind to MHC II alleles are selected for synthesis and/or coadministration to a subject in the second round of vaccination. In some preferred embodiments, at least 5 peptides that bind to MHC II alleles or nucleic acids encoding the peptides that bind to MHC II alleles are selected for synthesis and/or coadministration to a subject in the second round of vaccination. In some preferred embodiments, at least 10 peptides that bind to MHC II alleles or nucleic acids encoding the peptides that bind to MHC I alleles are selected for synthesis and/or coadministration to a subject in the second round of vaccination. In some preferred embodiments, at least 15 peptides that bind to MHC II alleles or nucleic acids encoding the peptides that bind to MHC I alleles are selected for synthesis and/or coadministration to a subject in the second round of vaccination. In some preferred embodiments, at least 20 peptides that bind to MHC II alleles or nucleic acids encoding the peptides that bind to MHC II alleles are selected for synthesis and/or coadministration to a subject in the second round of vaccination. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., at least 5 peptides that bind MHC I alleles and at least 5 peptides that bind MHC II alleles, and so on).

In some preferred embodiments, from 2 to 100 peptides that bind to MHC I alleles or nucleic acids encoding the peptides that bind to MHC I alleles are selected for synthesis and/or coadministration to a subject in the second round of vaccination. In some preferred embodiments, from 5 to 100 peptides that bind to MHC I alleles or nucleic acids encoding the peptides that bind to MHC I alleles are selected for synthesis and/or coadministration to a subject in the second round of vaccination. In some preferred embodiments, from 10 to 100 peptides that bind to MHC I alleles or nucleic acids encoding the peptides that bind to MHC I alleles are selected for synthesis and/or coadministration to a subject in the second round of vaccination. In some preferred embodiments, from 15 to 100 peptides that bind to MHC I alleles or nucleic acids encoding the peptides that bind to MHC I alleles are selected for synthesis and/or coadministration to a subject in the second round of vaccination. In some preferred embodiments, from 20 to 100 peptides that bind to MHC I alleles or nucleic acids encoding the peptides that bind to MHC I alleles are selected for synthesis and/or coadministration to a subject in the second round of vaccination. In some preferred embodiments, the allele is not A0201 or A2402. In some preferred embodiments, from 2 to 100 peptides that bind to MHC II alleles or nucleic acids encoding the peptides that bind to MHC II alleles are selected for synthesis and/or coadministration to a subject in the second round of vaccination. In some preferred embodiments, from 5 to 100 peptides that bind to MHC II alleles or nucleic acids encoding the peptides that bind to MHC II alleles are selected for synthesis and/or coadministration to a subject in the second round of vaccination. In some preferred embodiments, from 10 to 100 peptides that bind to MHC II alleles or nucleic acids encoding the peptides that bind to MHC I alleles are selected for synthesis and/or coadministration to a subject in the second round of vaccination. In some preferred embodiments, from 15 to 100 peptides that bind to MHC II alleles or nucleic acids encoding the peptides that bind to MHC I alleles are selected for synthesis and/or coadministration to a subject in the second round of vaccination. In some preferred embodiments, from 20 to 100 peptides that bind to MHC II alleles or nucleic acids encoding the peptides that bind to MHC II alleles are selected for synthesis and/or coadministration to a subject in the second round of vaccination. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., from 5 to 100 peptides that bind MHC I alleles and from 5 to 100 peptides that bind MHC II alleles, and so on).

In some preferred embodiments, from 2 to 50 peptides that bind to MHC I alleles or nucleic acids encoding the peptides that bind to MHC I alleles are selected for synthesis and/or coadministration to a subject in the second round of vaccination. In some preferred embodiments, from 5 to 50 peptides that bind to MHC I alleles or nucleic acids encoding the peptides that bind to MHC I alleles are selected for synthesis and/or coadministration to a subject in the second round of vaccination. In some preferred embodiments, from 10 to 50 peptides that bind to MHC I alleles or nucleic acids encoding the peptides that bind to MHC I alleles are selected for synthesis and/or coadministration to a subject in the second round of vaccination. In some preferred embodiments, from 15 to 50 peptides that bind to MHC I alleles or nucleic acids encoding the peptides that bind to MHC I alleles are selected for synthesis and/or coadministration to a subject in the second round of vaccination. In some preferred embodiments, from 20 to 50 peptides that bind to MHC I alleles or nucleic acids encoding the peptides that bind to MHC I alleles are selected for synthesis and/or coadministration to a subject in the second round of vaccination. In some preferred embodiments, the allele is not A0201 or A2402. In some preferred embodiments, from 2 to 50 peptides that bind to MHC II alleles or nucleic acids encoding the peptides that bind to MHC II alleles are selected for synthesis and/or coadministration to a subject in the second round of vaccination. In some preferred embodiments, from 5 to 100 peptides that bind to MHC II alleles or nucleic acids encoding the peptides that bind to MHC II alleles are selected for synthesis and/or coadministration to a subject in the second round of vaccination. In some preferred embodiments, from 10 to 50 peptides that bind to MHC II alleles or nucleic acids encoding the peptides that bind to MHC I alleles are selected for synthesis and/or coadministration to a subject in the second round of vaccination. In some preferred embodiments, from 15 to 50 peptides that bind to MHC II alleles or nucleic acids encoding the peptides that bind to MHC II alleles are selected for synthesis and/or coadministration to a subject in the second round of vaccination. In some preferred embodiments, from 20 to 50 peptides that bind to MHC II alleles or nucleic acids encoding the peptides that bind to MHC II alleles are selected for synthesis and/or coadministration to a subject in the second round of vaccination. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., from 5 to 50 peptides that bind MHC I alleles and from 5 to 50 peptides that bind MHC II alleles, and so on).

In some preferred embodiments, the peptides or nucleic acids encoding the peptides in the second round of vaccination have a combination of 2 or more or mutations selected from the group consisting of a missense mutation, an insertion mutation, a deletion mutation, an in-frame nucleotide mutation or out-of-frame nucleotide mutation.

In some preferred embodiments, the peptides or nucleic acids encoding the peptides are administered parenterally. In some preferred embodiments, the parenteral delivery is intradermal or subcutaneous. In some preferred embodiments, the peptides or nucleic acids encoding the peptides are administered orally.

In some preferred embodiments, the peptides or nucleic acids encoding the peptides are administered in a vaccine comprising an adjuvant.

In some preferred embodiments, the peptides or nucleic acids encoding the peptides are administered in vaccine via a viral vector.

In some preferred embodiments, the peptides or nucleic acids encoding the peptides are administered in a vaccine comprising a lipid drug delivery system.

In some preferred embodiments, the first or second vaccination is followed by a checkpoint inhibitor or other immunotherapeutic intervention to enhance T cell activity.

In some preferred embodiments, the methods further comprise a step of analyzing for adverse reactions based on T cell expose motif match in the human proteome.

In some preferred embodiments, the methods of the present invention further comprise harvesting antigen presenting cells and contacting the harvested cells in vitro with the one or more of the peptides, or nucleic acids encoding the same peptides identified in the methods described above. In some preferred embodiments, the methods further comprise reinfusing the antigen presenting cells into the subject from whom they were drawn. In some preferred embodiments, the methods further comprise contacting the antigen presenting cells which have been contacted by the peptides with T cells drawn from the subject and reinfusing the T cells into the subject.

In some preferred embodiments, the present invention provides a diagnostic test for monitoring the immune response to vaccination with any of the methods of claims 1 to 79 comprising a capture reagent selected from the group consisting of peptides encoded by SEQ IDs 1-537 and combinations thereof.

In some preferred embodiments, the present invention provides a vaccine comprising one or more synthetic peptides or nucleic acids encoding the synthetic peptides derived from VEGFR1 or VEGFR2 wherein the synthetic peptides bind one or more MHC alleles and the synthetic peptides are selected from the group consisting of SEQ ID NOs: 1-28 and combinations thereof. In some preferred embodiments, the present invention provides a vaccine comprising one or more synthetic peptides or nucleic acids encoding the synthetic peptides derived from vimentin wherein the synthetic peptides bind one or more MHC alleles and the peptides are selected from the group consisting of SEQ ID NOs: 29-103 and combinations thereof. In some preferred embodiments, the present invention provides a vaccine comprising one or more synthetic peptides or nucleic acids encoding the synthetic peptides derived from WT-1 wherein the synthetic peptides bind one or more MHC alleles and the peptides are selected from the group consisting of SEQ ID NOs: 104-118 and combinations thereof.

In some preferred embodiments, the peptides in the vaccine bind one or more MHC I alleles. In some preferred embodiments, the peptides do not bind MHC I alleles A0201 or A2402 at less than 200 nM. In some preferred embodiments, the peptides are 8-10 amino acids. In some preferred embodiments, the peptides bind one or more MHC II alleles. In some preferred embodiments, the peptides are 15-18 amino acids. In some preferred embodiments, the predicted binding affinity of the peptides to the one or more MHC alleles is less than 20 nanomolar. In some preferred embodiments, the predicted binding affinity of the peptides to the one or more MHC alleles is less than 50 nanomolar. In some preferred embodiments, the predicted binding affinity of the peptides to the one or more MHC alleles is less than 200 nanomolar. In some preferred embodiments, the vaccines further comprise a pharmaceutically acceptable carrier. In some preferred embodiments, the vaccines further comprise an adjuvant.

In some preferred embodiments, the present invention provides a vaccination regimen comprising administering a vaccine as described above to a subject with cancer.

In some preferred embodiments, the present invention provides a vaccine as described above for use in treating cancer or tumor in a subject. In some preferred embodiments, the tumor is a tumor of a tissue selected from the group consisting of nervous system, lung, breast, pancreas, liver, skin, prostate, genito-urinary tract, hematopoietic system, gastro-intestinal, endocrine system, and musculoskeletal tissues. In some preferred embodiments, the tumor of the nervous system tissue is selected from the group consisting of glioma, glioblastoma, neuroblastoma, meningioma, schwannoma, and metastases to the brain from other sites.

### DESCRIPTION OF THE FIGURES

FIG. 1: Overview of the immunotherapeutic strategy.

FIG. 2: VEGFR1: Overview of MHC binding, B cell epitopes and topology. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The kblu line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 3: VEGFR2: Overview of MHC binding, B cell epitopes and topology. Legend as for FIG. 1.

FIG. 4: Vimentin Overview of MHC binding, B cell epitopes and topology. Legend as for FIG. 1.

FIG. 5: Wilms tumor protein WT1 Overview of MHC binding, B cell epitopes and topology. Legend as for FIG. 1.

FIG. 6: EGFR and EGFRvIII. Overview of MHC binding, B cell epitopes and topology. Legend as for FIG. 1.

FIG. 7: Few MHC I alleles bind naturally at each of the five unique TCEM positions I each common EGFR mutant. The figure highlights those binding at better than 1 SD units below the mean for the protein (approx. 500 nm).

FIG. 8: Isocitrate dehydrogenase 1 (IDH1 mutant R132H). Overview of MHC binding, B cell epitopes and topology. Legend as for FIG. 1.

FIG. 9: Histone 3.3 Overview of MHC binding, B cell epitopes and topology. Legend as for FIG. 1.

FIG. 10: BRAF Overview of MHC binding, B cell epitopes and topology. Legend as for FIG. 1.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (on the world wide web at ebi.ac.uk/interpro). Human proteome is also understood to include those proteins and antigens thereof which may be over-expressed in certain pathologies or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general, “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response or may result in down regulation or immunosuppression. Thus, the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. The B cell epitope may be evaluated by predicting the binding of across a series of 9-mer windows, the core pentamer then is the central pentamer of the 9-mer window

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally.

As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, an “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or‘Q’. The suffix‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off-rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “Ka”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷M⁻¹ to 2×10⁶M⁻¹.

As used herein, the terms “weak binder” and “weak binding” and “low affinity” refer to a binding pair or describe a binding pair that have an affinity of less than 2×10⁶M⁻¹ (equivalent to a dissociation constant of 500 nM Kd)

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to enhance the immunological response, depending on the host species, including but not limited to Freund's (complete and incomplete), mineral gels such as aluminum hydroxide, surface active substances such as lysolecithin, Lipid A analogues (e.g. poly I:C), pluronic polyols, polyanions, peptides, oil emulsions, CpG, C type lectin ligands, CD1d ligands (e.g. α-galactosylceramide), squalene, squalene emulsions, liposomes, imidazoquinolines (e.g. imiquimod), keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.). A “subject affected by cancer” is a cancer patient.

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1, 2, 3, 9). In the case of MHC-II molecules two formats of GEM are most common comprising amino acids (−3, 2, −1, 1, 4, 6, 9, +1, +2, +3) and (−3, 2, 1, 2, 4, 6, 9, +1, +2, +3) based on a 15-mer peptide with a central core of 9 amino acids numbered 1-9 and positions outside the core numbered as negative (N terminal) or positive (C terminal).

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“T-cell exposed motif” (also where abbreviated TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“Isoform” as used herein refers to different forms of a protein which differ in a small number of amino acids. The isoform may be a full-length protein (i.e., by reference to a reference wild-type protein or isoform) or a modified form of a partial protein, i.e., be shorter in length than a reference wild-type protein or isoform.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances, such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs.

Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is one which is completely missing in the human proteome or present in up to only five instances in the human proteome.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

“Immunoglobulinome” as used herein refers to the total complement of immunoglobulins produced and carried by any one subject.

As used herein the term “repertoire” is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion. Mutated amino acid as used herein thus refers to either a substitution of one amino acid for another or to the novel juxtaposition of two amino acids as the result of an insertion, deletion, splice variant or fusion. Hence, the EGFR splice variant vIII is considered a “mutated amino acid” herein.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non-limiting example, a programmed death protein of which there are multiple isoforms.

As used herein “immunotherapy intervention” is used to describe any deliberate modification of the immune system, including but not limited to, through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed on the world wide web at uniprot.org/docs/cdlist.

As used herein “tumor associated antigens” “TAA” and “tumor associated antigen proteins” refers to proteins which are upregulated in a tumor, but not necessarily mutated relative to that protein in normal tissue. Such tumor associated antigens comprise those that are common to many cases of the same type of cancer and those that are unique to the individual subject. Some tumor associated antigens are derived from viruses such as, but not limited to, herpesviruses, papillomaviruses, retroviruses and polyomaviruses.

As used herein “presentome” refers to the peptides bound in MHC and presented on the surface of antigen presented cells. Mass spectroscopy detects some but not all peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

“Tumor specific antigen” or “tumor specific epitope” is used herein to designate an epitope or antigen that differentiates a mutated tumor protein from its unmutated wildtype homologue. Thus, a neoantigen is one type of tumor specific antigen.

As used herein “driver mutations” are those which arise very early in tumorigenesis and are causally associated with the early steps of cell dysregulation. Driver mutations are shared by all clonal offspring arising from the initial tumor cells and offer some additional fitness benefit to the clonal line within its microenvironment. In contrast “passenger mutations” are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver” and “passenger” are terms largely interchangeable with “trunk” and “branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations. A bespoke peptide or bespoke vaccine is also referred to herein as a “personalized peptide”, “personalized peptide vaccine”, “personalized neoepitope vaccine” or “personalized vaccine”.

As used herein “TCGA” refers to The Cancer Genome Atlas (one the world wide web at cancer.gov/about-nci/organization/ccg/research/structural-genomics/tcga).

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Representative autoimmune diseases include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis, Bullous Pemphigoid, Castleman's disease, Celiac disease, Cogan syndrome, Cold agglutinin disease, Crohn's Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis.

“Antigen presenting cell” as used herein refers to cells which are capable of presentation of peptides to T cells bound to MHC molecules. This includes but is not limited to the so called “professional” antigen presenting cells comprising, but not limited to, dendritic cells, B cells, and macrophages, but also the so called non-professional antigen presenting cells which carry MHC molecules.

“Cognate” as used herein in the context of a T cell refers to the T cell capable of binding a particular epitope.

“Vaccination round” or “round of vaccination” as used herein refers to a vaccination regimen which may comprise one or more administrations of the same one or more immunogens. Hence, a primary immunization followed by one or more boosters with the same antigen is considered a “round” of vaccination.

“Surgical intervention” as used herein in the context of a tumor refers to any intervention by a surgeon which may result in the resection of tissue. It does not refer to simple needle biopsies conducted in an outpatient clinic.

“Genome Data Commons” as used herein refers the National Cancer Institute database maintained at the University of Chicago and on the world wide web at portal.gdc.cancer.gov/ and gdc.cancer.gov/.

“Mutational landscape” is a term used herein to describe the total array of mutations found in any one individual cancer subject or characteristic of a particular type of cancer.

“Lipid drug delivery system” or LDDS as used herein is a generic term which encompasses lipid nanoparticles, emulsions, self-emulsifying drug delivery systems, nanocapsules and liposomes, wherein molecules of a drug active product is encased or partially encased in lipid.

“HUGO” as used herein refers to the Human Genome Organisation Gene Nomenclature Committee at the European Bioinformatics Institute (one the world wide web at genenames.org) which assigns a name and an approved gene symbol to each gene. Examples of HUGO gene names included herein are EGFR (Epidermal growth factor receptor), H3.3 or H33 (Histone H3.3), IDH (isocitrate dehydrogenase), BRAF (Serine/threonine-protein kinase B-raf), TP53 (Cellular tumor antigen p53), PTEN (Phosphatidylinositol 3,4,5-trisphosphate 3-phosphatase and dual-specificity protein phosphatase), ERBB2 (Receptor tyrosine-protein kinase erbB-2), PIK3CA (Phosphatidylinositol 4,5-bisphosphate 3-kinase catalytic subunit alpha isoform), and KRAS (GTPase KRas). Other examples which are found in fusion proteins mentioned herein are KIAA1549-BRAF (UPF0606 protein KIAA1549 fused to Serine/threonine-protein kinase B-raf) and EML4-ALK (Echinoderm microtubule-associated protein-like 4 fused to ALK tyrosine kinase receptor).

### DESCRIPTION OF THE INVENTION

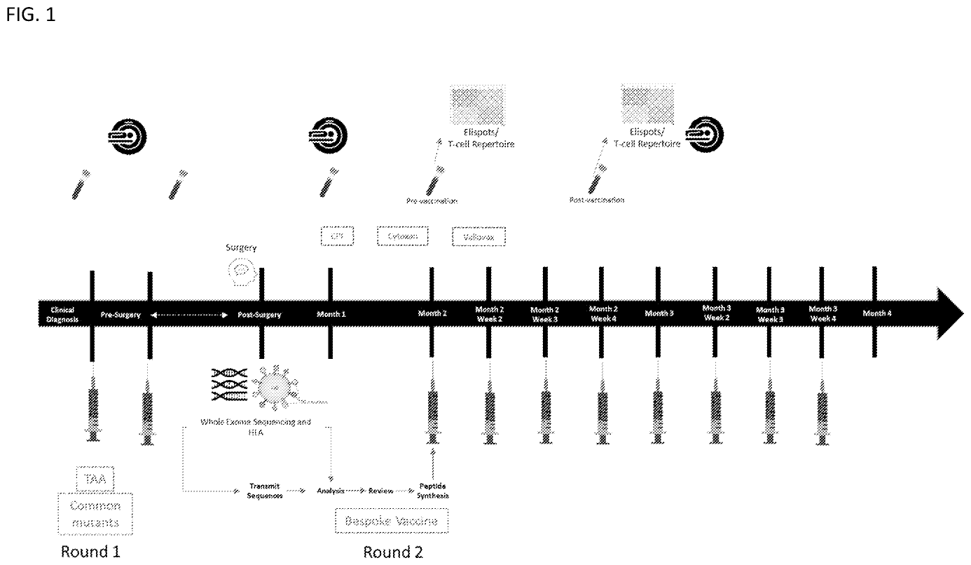

This invention provides a method for stimulating the immune response to a tumor by implementing a two-phase vaccination strategy as shown in FIG. 1. A first vaccination comprises either or both of epitopes from tumor associated antigens, or epitopes which embody amino acid mutations commonly associated with the clinically diagnosed cancer, wherein the first vaccination is administered before tissue diagnosis. The first vaccination intervention, in preferred embodiments, is conducted prior to other immunotherapeutic interventions once there is a clinical or imaging diagnosis, before any surgery. A second vaccination comprises personal neoepitopes, the design of which is unique to that subject and it is designed based on comparative sequencing of normal tissue and tumor tissue obtained by biopsy. In preferred embodiments the second vaccination follows surgical intervention to resect the tumor or obtain a biopsy and identification of actionable mutations in proteins expressed in the tumor. Each of the vaccinations may comprise multiple administrations of that vaccinal composition.

The stimulation of the immune system is achieved either by means of stimulation of dendritic cells or T cells in vitro followed by administration of these cells to a patient, or by means of administration directly to the subject of a vaccine in which peptides, or their encoding nucleic acids, have been selected or designed to ensure an appropriate level of binding affinity to the particular cancer patient's MHC alleles. The first and second rounds of vaccinations can be administered with a variety of adjuvants and other immunostimulatory agents, not limited to checkpoint inhibitors, radiation, chemotherapy and targeted drugs, and may be administered by any of a number of routes.

In preferred embodiments the HLA alleles of the subject are determined prior to the first round of vaccination. Once the HLA alleles of the subject are known, in preferred embodiments the design of the peptides, or their encoding nucleic acids, from the common mutations may be modified to achieve a desired binding affinity to the alleles. This is achieved by maintaining the mutated amino acids withing the T cell exposed motif, while substituting amino acids in the groove exposed motif to achieve the desired binding affinity. The same process of optimization of binding affinity may be conducted for the personalized peptides of the second vaccination.

While cancer arises as the result of gene mutations which produce dysregulation of growth [1], it is also the consequence of immune evasion. Immune evasion may be the result of exhaustion or suppression in the tumor microenvironment [2]. Immune evasion may also arise because of the absence of cognate tumor-specific T cells in the T cell repertoire. Indeed, our prior characterization of tumor mutations indicates that mutations are often concealed from T cells by poor, or at less non-competitive, binding to MHC molecules and preferential location in pocket or groove exposed positions where they are not able to engage a T cell receptor (See, e.g., PCT US2020/037206, incorporated by reference herein in its entirety). In addition, tumor mutations often give rise to rare amino acid sequence motifs, not otherwise present in the human proteome, which are more likely to lie outside the subject's repertoire of cognate T cells. Immunotherapy interventions, such as checkpoint inhibitors and other immunotherapeutic agonists [3] which enhance and activate T cells responses have brought a significant change to the prognosis of cancer patients. However, checkpoint inhibitors are still limited in their efficacy to about 25-30% of cases [4]. Efficacy is higher in subjects and tumor types with a higher number of mutations [5, 6] where the probability of unleashing an effective tumor specific cognate T cell is higher. Checkpoint inhibitors activate the T cells that a subject already has, but if a cognate T cell clone is absent and thus cannot be upregulated, a checkpoint inhibitor may be limited in efficacy. Thus, it is a desirable goal to increase the number of T cell clones which are directed to epitopes on the tumor cells, and ideally to do this as early as possible in case management. The present invention is directed to this goal of maximizing tumor specific T cell clones, and in particular active CD8+ cytotoxic T cells, as early and rapidly as possible in the course of disease. Maturation and establishment of memory of CD8+ T cells is only completed with the help of CD4+ T helper cells [7]. Therefore, each antigen directed to stimulate CD8+ cells is ideally accompanied by an antigen that will stimulate a close companion CD4+ cell clonal line.

The approach described herein therefore addresses the phased combination of two therapeutic strategies: firstly, early intervention based on clinical diagnosis, and secondly, personalization of neoepitope vaccines following tumor exome sequencing and comparison of tumor vs normal gene mutations.

### Early Intervention

As the sophistication of sequencing of tumors has advanced, the gene mutations most commonly associated with certain cancers have become better understood. Although common driver mutational patterns are now better recognized, each cancer is different and adopts a pattern of additional mutations that is “personal” to the particular subject. This is a combination not only of the stochastic nature of mutations, particularly passenger mutations occurring as the cancer develops, but also the immune pressure created according to the subject's particular HLA alleles and the unique composition of that subject's T cell repertoire and hence the gaps in that repertoire which may facilitate the survival of a cancer cell carrying a particular mutation or array of mutations. However, full characterization of the tumor mutations, their protein coding consequences and the level of expression of such proteins, cannot be evaluated without access to a biopsy. Such a biopsy is obtained through a surgical procedure at a time point determined by clinical progression. Completing exome sequencing of DNA and RNA from a biopsy is relatively rapid with current techniques but may still lead to a lapse of a few weeks before a personalized vaccine plan can be designed following collection of a biopsy, during which time the tumor continues to grow unchecked. Thus, it is desirable to jumpstart the immune response earlier in anticipation of a complete personalized vaccine at a later date. Furthermore, the surgical intervention itself may result in greater exposure to T cells of epitopes derived from damaged tissue. Thus, priming T cells to the degree possible prior to surgery is desirable to enhance the number of T cell clones cognate to the tumor epitopes as early as possible.

Two groups of tumor epitopes are useful for early stimulation: common mutations found in the cancers of the particular clinical presentation and certain non-mutated tumor associated antigens.

### Mutations Common to the Clinical Presentation

While cancer mutations arise stochastically, certain mutations have emerged as characteristic of certain types of cancer. These are typically in driver genes which determine the pathway of dysregulation of the cancer. As such mutations are indeed tumor specific and are not found in normal tissue, it is possible to target them with relatively low risk of directing an autoimmune response to the corresponding normal protein homolog. The utilization of short peptide immunogens further reduces the possibility of an off-target reaction by minimizing the number of T cell exposed motifs to those essential to generate the tumor-specific response sought. This is the case both to avoid adverse targeting of the homolog normal protein, but also to avoid targeting unrelated proteins which may carry, and present, the same T cell exposed motif. As discussed in Example 11 a method has been developed (See, e.g., PCT US2020/037206, incorporated herein by reference in its entirety) for review of potential off-target autoimmune responses, allowing risk benefit analysis, and mitigating the risk of autoimmunity.

A number of mutations that are highly characteristic of certain cancer types can thus be considered for targeting upon initial clinical presentation. Selected peptides embodying such common mutations, or their encoding nucleic acids, can be applied as a vaccine with a high probability that they will be present but with minimal risk of adverse consequences if they are not present. In order to render a tumor specific response and minimize off target reactions it is necessary to select the peptide antigens so as to have the mutant amino acids exposed to the T cell receptor, i.e. with the mutant amino acids in the T cell exposed motifs. Peptide vaccines, or vaccines comprising peptides encoded in nucleic acid or vectors, directed to the most common mutated proteins, and designed for MHC binding with each HLA allele in mind, can be prepared and held “ready to go” for early intervention as cases present for initial clinical evaluation.

Examples of common mutations which are suitable for such early intervention use are as follows. These are illustrative examples and thus considered non limiting.

In glioblastoma, over half of the cases carry aberrant epidermal growth factor receptor (EGFR). Approximately 30% show upregulated EGFRvIII, a splice variant lacking exons 2-7 [3, 8]. In addition, about 25% cases have mutations in the extracellular domain of EGFR including A289V/D/T, R108K, and G598D. Conversely in lung cancer the most common mutation of EGFR is L858R. While prior efforts have been made to target the EGFRvIII by vaccination [9], these adopted a strategy designed to stimulate antibody dependent cell mediated cytotoxicity, and by linking the EGFR target peptide to keyhole limpet hemocyanin, a very strong and dominant T helper epitope which is likely to out-compete the formation of tumor specific T cells. In one embodiment therefore, described in Example 4 and 5, we provide examples of epitope peptides for exemplar HLA alleles which can target the tumor specific motifs of EGFRvIII and the most common mutations in EGFR.

Midline gliomas are frequently characterized by mutation of histone H3 variant proteins and in particular a K27M mutation of H3.3. While peptide approaches have been proposed, these have been restricted to subjects carrying HLA A0201 and one particular peptide RMSAPSTGGV [10, 11] (see also U.S. Pat. No. 10,441,644). While this peptide has a moderately high predicted binding affinity to A0201, the location of the mutant methionine at position 2 means that this amino acid is preferentially hidden in a pocket position of the MHC groove (groove exposed position) and thus unlikely to stimulate a T cell response that will effectively differentiate tumor and normal tissue. In the present invention, by using modifications of the groove exposed motif of peptides in which the mutant methionine is exposed in the T cell exposed motif, we identify other peptides which are capable of directing a CD8+ response to the tumor and for other HLA alleles.

A very common missense mutation in isocitrate dehydrogenase R132H is often found in gliomas and glioblastomas as well as several other cancers, and is associated with hypermethylation leading to genetic instability which may trigger other mutations [12]. Mutations in isocitrate dehydrogenase enzyme isoform 1 (IDH1) and, to a lesser extent in isoform 2 (IDH2) genes have been identified in a large proportion of diffuse astrocytomas (70-90%), oligodendrogliomas (69-94%), oligoastrocytomas (78-100%), and secondary glioblastomas (82-88%). Peptides have been identified in the vicinity of the mutation in IDH which can stimulate CD4+ cells and produce a tumor suppressive effect [13, 14] (see also U.S. Ser. No. 10/161,940). In the present invention we identify tumor specific epitopes in IDH R132H that can target an array of CD8+ cells in subjects of differing HLA alleles and also provide examples of personalized peptides with altered groove exposed motifs to optimize binding for particular HLAs.

Two mutations are highly characteristic of pediatric low-grade gliomas: KIAA1549-BRAF and BRAFV600E. In one study these together accounted for 68% of pediatric low-grade gliomas [15]. At present only two forms (long/short) of fusion of KIAA1549 to BRAF are described, providing unique neoepitopes at the fusion junction. BRAF mutations at position 600 are common not only in this cancer but in many others, including but not limited to thyroid, skin (melanoma) many adenocarcinomas and others. BRAF600 mutations are among the most common recorded at the Genome Data Commons, especially V600E, but also at lower frequency V600G, and V600M. The present invention provides peptides which can be utilized to target tumor specific T cell exposed motifs harboring such common BRAF mutations.

The above non-limiting examples are provided to indicate that based on clinical presentation, a probable occurrence of certain common mutations can be determined. This can be further validated by imaging studies with radiolabeled tracers (e.g. EGFR) and molecular- and immuno-diagnostics conducted on blood/CSF/urine/saliva, etc. (liquid biopsies), needle aspirates, or bone marrow samples prior to access to a tumor biopsy (for instance for IDH mutations, EGFR).

### Tumor Associated Antigens Relevant to Clinical Presentation

Immune responses are recognized to a number of proteins that are upregulated in association with tumors or which may control the progression of tumors. A large number of tumor associated antigens have been identified and ranked according to their utility as tumor target antigens for immunotherapy [16]. Among the most prominent tumor associated antigens with immunotherapeutic potential are WT1, MUC1, LMP2, HPV E6 E7, EGFRvIII, HER-2/neu, MAGE A3, p53, NY-ESO-1, PSMA, GD2, CEA, MelanA/MART1, Ras-mutant, gp100, Proteinase3 (PR1), Bcr-abl, Tyrosinase, Survivin, PSA, hTERT, Sarcoma translocation breakpoints, EphA2, PAP, ML-IAP, AFP, EpCAM, ERG (TMPRSS2 ETS fusion gene), NA17, PAX3, ALK, Androgen receptor, Cyclin B1, Polysialic Acid, MYCN, RhoC, TRP-2, GD3, Fucosyl GM1, Mesothelin, PSCA, MAGE A1, sLe(a), CYP1B1, PLAC1, GM3, BORIS, TBXT (brachyury), BCAN, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, VEGFR2, MAD-CT-1, FAP, PDGFR-beta, MAD-CT-2, and Fos-related antigen 1. These tumor associated antigens (TAA) were among the first cancer immunogens to be explored, albeit with limited success [17-19]. In some instances, there was insufficient understanding of the precise nature of MHC allele-specific presentation. In other cases, the epitopes were not well characterized. Many trials of TAA vaccination preceded the era of checkpoint inhibitors. There are, however, examples of beneficial effects of vaccination with tumor associated antigens [18, 20-23]. In the present invention we provide a method for combining the vaccination with certain epitope peptides from tumor associated antigens with subsequent vaccination with a personalized cancer vaccine.

While any of the above listed tumor associated antigens offer opportunities to target cancer cells and in the present invention are considered targets for the first round of vaccination, some particular examples of actionable tumor associated antigens which are suitable for such early intervention use are as follows. These are illustrative examples and thus considered non limiting.

Vascular endothelial growth factor receptor 1 (VEGFR1) and Vascular endothelial growth factor receptor 2 (VEGFR2), acting as receptors for vascular endothelial growth factors A and B, play a key role in the regulation of angiogenesis, cell migration, and cancer cell invasion. The benefit of reducing vascularization of tumors has been shown by bevacizumab, a monoclonal antibody to VEGFA. Similarly inducing an immune response to VEGFR 1 and 2 has been successful in reducing tumorigenesis in multiple cancers and in mitigating choroidal vascularization [20, 21, 24] (see also U.S. Pat. No. 8,975,229). However, these studies have focused exclusively on two HLA alleles A0201 and A2402 and do not teach how to apply immunization to VEGFR to patients of other alleles. A Salmonella vectored VEGFR protein has also been successfully delivered orally to reduce progression of pancreatic cancer [25]. The present invention provides VEGFR peptides which can be applied more broadly to other subjects with other HLA alleles. In some preferred embodiments such administration is early in the clinical course of the cancer and is followed by a personalized neoepitope vaccination.

Vimentin is a filamentous cytoskeleton protein expressed in most mesenchymal cells. Autoantibodies to post translationally citrullinated vimentin have been linked to rheumatoid arthritis and sarcoidosis. Autoantibodies to vimentin are also documented in systemic lupus erythematosus [26]. In adenocarcinoma cancer cells, including gliomas, glioblastomas and melanoma, vimentin adopts a different structural configuration which exposes an ectodomain, which has served as the target of the therapeutic antibody pritumumab [27-29]. The B cell conformational epitope is not found on normal cells, although the sequence of vimentin remains unchanged. As T cell epitopes are not subject to the same structural constraints as antibody epitopes, it follows that the T cell epitopes of vimentin offer a therapeutic vaccine target. In the present invention, therefore, peptides comprising T cell epitopes in the ectodomain of vimentin are among those provided as an early intervention. In the examples cited below we provide predicted T cell epitopes in vimentin for multiple HLA alleles including both MHC I and MHC II.

Wilms tumor antigen (WT1) is expressed in many cancers, having originally being identified in a childhood renal cancer, Wilms tumor. In addition to renal cancer, WT1 is recognized in many different hematologic cancers, lung cancer, pancreatic cancer, melanoma and a variety of adenomas and adenocarcinomas. It encodes a zinc finger transcription factor that is critical to cell differentiation and it is considered an oncogene [30, 31].

WT-1 has long been recognized as a tumor associated antigen with therapeutic potential [16, 30], however while peptides from this protein have been used in vaccine studies of pancreatic and hematologic cancers, they have been restricted only to those peptides which bind to A0201, A2402 and A0206. Peptides with wider applicability across a greater diversity of the population have not been evaluated [30, 32-34]. In addition a peptide with MHC II binding capable of providing T cell help was identified [35]. In the present invention we provide examples of peptides with broader MHC binding and with suitability for vaccination of individuals with a broad diversity of MHC I and MHC II alleles. While there are multiple isoforms of WT1, the examples below show, it is possible to identify epitopes in conserved regions with broad allele binding.

Tumor associated antigens also include viral antigens. In some particular embodiments the first round of vaccination may include epitope peptides, or their encoding nucleic acids, derived from oncogenic viruses including, but not limited to, human papillomaviruses and herpesviruses. The latter may include epitopes from, but not limited to human herpesviruses 1 and 2 (herpes simplex virus), human herpesvirus 4 (Epstein Barr virus) and human herpesvirus 5 (human cytomegalovirus) [36]. Certain viruses are known to be frequently upregulated in certain cancers, for example human cytomegalovirus in glioblastoma. Whether this is a driver or a consequence of the tumorigenesis is not clear, but the presence of the CMV antigens provide a way to target cancer cells [37, 38]. While such viruses may play a role in tumorigenesis and share common antigens which can be targeted early in the clinical course as provided here in a first round of vaccination, the resultant tumors each have diverse mutations which are then targeted in a second personalized vaccination strategy in round two as described below.

### Personalized Cancer Vaccines

There is increasing evidence that a variety of T cell immunotherapies can be successful in halting the progression of cancer [39].

Tumor-specific antigens comprise both those common to many cancers, and those which are unique to any single patient and which may change over the life of a tumor. Generally, the higher the mutational load, the more infiltrating T cells and the more inflamed a tumor, the greater probability there is of a check-point inhibitor leading to a successful T cell driven elimination of the tumor cells. Mutational load tends to differ between cancer types; some such as melanoma and colorectal cancers have a high mutational frequency. Others such as glioblastoma are notoriously low in mutational numbers.

Several recent publications have reported promising, but mixed, results in the development of personalized vaccines for melanoma [40, 41], lung cancer [42, 43] and glioblastoma [44, 45]. These have employed from 1 to 20 different neoantigens. Increasing the number of neoepitopes incorporated in a vaccine allows for a multipronged attack on the tumor using multiple alleles and multiple antigens derived from different proteins. Mutations continue to arise in tumors as they develop, with antigens gained or lost in the process. There may also be heterogeneity of mutations within a tumor and the mutational landscape may not be fully reflected in the sequencing of a biopsy. Hence a high number of cytotoxic “hits” is desirable rather than depending on only one or two antigen targets [46]. A goal of the present invention is to maximize the number of tumor specific epitopes which can be targeted by T cells responding to peptides presented by a particular patient's alleles.

The goal of T cell immunotherapy has been primarily to activate CD8+ cytotoxic T cells which will target tumor cells, but also to stimulate CD4+ T helper cells to enhance CD8+ responses. Stimulation of CD4+ T helper cells may also enhance B cell responses. Selection of peptides for use as neoepitopes has followed several paths. As a starting point, given the diversity of the human genome, it is desirable to compare sequences of proteins in tumor biopsies with a normal tissue from the same patient [47]. However, reference human genomes are frequently used as comparators to determine mutation sites. Practitioners have then used several approaches to select peptides for use, or for encoding in RNA or DNA for administration. In some instances peptides have been selected based on mass spectroscopy [48, 49]; in yet others predictive algorithms, most often NetMHC Pan [50], was used to select peptides [40, 41, 45]. In one instance, both approaches were reported, but in this particular case none of the mutated peptides were detected by mass spectroscopy [44].

Mutations in cancers include those which are unique to a specific patient. Some are patient specific driver mutations, arising as the root cause of cell dysregulation. Others arise as branch or passenger mutations, which are sequelae to an earlier trunk or driver mutation. Such mutations may continue to evolve throughout the tumor progression. There are also a number of mutations which are found commonly at the same positions in the same proteins, some of which occur repeatedly across many cancer types [51-55]. The Cancer Genome Atlas documents many proteins which are found to share mutations across multiple cancer types (on the world wide web at cancer.gov/about-nci/organization/ccg/research/structural-genomics/tcga). Some of these are simple amino acid substitutions arising from single nucleotide mutations; others involve amino acid duplications. In other cases, the mutations give rise to insertions and deletions (indels) and missense sequences. Gene fusions are another source of novel tumor specific motifs. Where these mutations are shared across many cancers, a set of peptides can be designed for each patient HLA allele which will allow stimulation of T cells to specifically target tumor cells with cytotoxic T cells and/or T helper cells.

In the case of indels and missense mutations, when these result in an in-frame downstream sequence they may provide a target-rich sequence, but every patient is unique and so selection of vaccine peptides for these must be handled as a personalized design effort. In some embodiments consistent indels are found repeatedly in many cancers.

In the present invention, a second round of vaccination is based on information gathered following collection of a biopsy of the tumor and a comparative sample of normal tissue. In some preferred embodiments, mutated proteins in the biopsy samples are identified by sequencing the genome, proteome, or transcriptome of cells from the biopsy, in other words obtaining DNA and RNA sequences and deducing protein sequences. The present invention is not limited to any particular method of obtaining sequences of proteins mutated in a biopsy. A variety of sequencing methods are readily available to those of ordinary skill in the art.

In some preferred embodiments, the present invention utilizes nucleic acid sequencing techniques. The nucleic acid sequences are preferably converted in silico to protein sequences from the identification of mutated amino acids and peptides comprising the mutated amino acids.

In some embodiments, the sequencing is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing. In some embodiments, the sequencing is parallel sequencing of partitioned amplicons (PCT Publication No: WO2006084132 to Kevin McKeman et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotiter plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, sequencing is nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, sequencing is HeliScope by Helicos BioSciences (Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb to 100 Gb generated per run. The read-length is 100-300 base pairs. The accuracy for homopolymer repeats of 5 repeats in length is ˜98%. The benefits of ion semiconductor sequencing are rapid sequencing speed and low upfront and operating costs.

In some embodiments, sequencing is the technique developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety. Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In other preferred embodiments, the present invention utilizes protein sequencing techniques. In some embodiments, proteins may be sequenced by Edman degradation. See. e.g., Edman and Begg (1967). “A protein sequenator”. Eur. J. Biochem. 1 (1): 80-91; Alterman and Hunziker (2011) Amino Acid Analysis: Methods and Protocols. Humana Press. ISBN 978-1-61779-444-5. In other embodiments, mass spectrometry techniques are utilized to sequence proteins. See, e.g., Shevchenko et al., (2006) “In-gel digestion for mass spectrometric characterization of proteins and proteomes”. Nature Protocols. 1 (6): 2856-60; Gundry et al., (2009) “Preparation of proteins and peptides for mass spectrometry analysis in a bottom-up proteomics workflow” Current Protocols in Molecular Biology. Chapter 10: Unit 10.25.

Best practices in identifying tumor specific mutations have been described [56] although variations of the approach described are valid. In preferred embodiments described herein MHC binding, cathepsin cleavage patterns, and patterns of T cell exposed frequency are determined by mathematical algorithms previously described, see. e.g., PCT US2011/029192, PCT US2012/055038, US2014/01452, PCT US2020/037206, U.S. Pat. Nos. 10,706,955 and 10,755,801, each of which is incorporated by reference herein in its entirety.

Tumor Fraction and RNA Expression.

In order to effectively target an immune response to any individual tumor-specific mutation, a minimum of two initial conditions must be fulfilled: The mutation must be present in the DNA of a sufficient fraction of tumor cells and the DNA encoding the mutation must be transcribed into RNA and expressed as a protein. The tissue fraction comprising the mutant DNA can vary with the precision of resection of the biopsy and the relative composition of tumor to stromal tissue. It may also vary between metastases. In some instances, the fraction of the tumor biopsy comprising the mutation may be very high, represented in over 35% or over 50% of all cell DNA. In other instances, it may be lower, from 1-2% to 10%. In preferred embodiments the targeted mutations are selected from those mutant genes represented in at least 10% of the tumor DNA.

The RNA expression is an indicator of whether the gene is transcribed and hence actually targetable as a protein. Bulk RNA content of the tumor is enumerated and for each protein is normalized for the number of total reads of RNA detected in the biopsy sample and the length of the RNA transcript as a metric for gene expression. The number of RNA transcripts varies widely between proteins and overall the bulk RNA frequencies can be described as a log-normal distribution. If the gene is being expressed by both parental chromosomes, the relative expression of the normal and mutant allele for a mutated proteins should be correlated to the DNA mutation frequency. Allele specific expression has been shown to occur in tumors. Such a situation would be manifest with the parental chromosomes being expressed at differential rates and would lead to RNA mutant frequencies that differ from the frequency in the DNA. The mutation:normal ratio of expressed RNA compared to the DNA in the tumor fraction is an indicator of this. In some embodiments the DNA mutation occurs in only one chromosome and if expression of the protein is occurring solely or predominantly from the other chromosome, the mutant protein is not expressed or is under represented, rendering it an ineffective target.

To effectively target a tumor specific mutation it is necessary to establish that the protein is indeed being expressed from the parental chromosome containing the mutant gene. Methods for determination of the RNA fraction are described in Example 12 below. Thus in preferred embodiments peptides comprising mutant amino acids are selected from those proteins that are expressed and for which the RNA mutant: normal fraction is at least 10% of the corresponding DNA tumor:normal fraction.

### Considerations in Selection of Tumor Specific Antigens

Based on comparative sequencing of tumor biopsy and normal tissue a personalized set of neoepitopes can be defined that can form a personalized vaccine applied in the second round of vaccination. Some consideration in developing such a personalized vaccine are as follows.

### T Cell Exposed Motifs

The goal of stimulating a cytotoxic T cell response to a tumor is to specifically and differentially destroy the tumor cells while leaving normal cells intact. It follows that to drive a T cell response specific to the cancer, the T cell receptor must recognize an epitope unique to the tumor. Thus, the mutated amino acid must be located in the exposed pentameric motif exposed to the T cell receptor. When a mutated amino acid is located in a pocket or groove exposed motif, it may or may not affect binding affinity, but it is hidden from the T cell receptor and cannot elicit tumor-specific T cell responses. In some instances, the natural binding affinity of the mutated peptide and its neighboring peptides in the affected protein may give rise to better binding in positions which do not expose the mutated amino acid. In some cases, so-called neoepitope peptides have been selected which do not, in fact, differentiate tumor and normal T cell exposed motifs [42, 57]. In the present invention we seek to maximize use of the T cell exposed motifs containing mutant amino acids, and hence focus the T cell response on these differentiating epitopes, and likewise subsequent expansion of this response as the result of administration of checkpoint inhibitors.

### Peptide Binding Affinity

Many investigators have considered how to identify peptides in mutated tumor proteins which bind to a patient's MHC alleles. Some have employed mass spectrometry to identify the “presentome” of peptides bound and presented to T cells [48]. However, this has the bias of identifying very high affinity peptides. In some cases, the peptides containing mutant amino acids were never detected by mass spectroscopy [44].

It is not clear that the highest binding peptides are those which will actually generate the best cytotoxic T cell response. Indeed, evidence in other settings suggests that this is not the case and that an intermediate binding affinity may be most effective in stimulating a T cell response and good memory T cells [58]. Low affinity peptides may initiate a CD8+ response but this is not sustained [59]. Furthermore, also drawing on experience in an anti-microbial setting, an active interferon gamma response is also needed to trigger the development of T memory cells [60]. Strength of T cell receptor—pMHC binding may be a factor in determining whether the T cell response to a tumor leads to T cell exhaustion and tolerance [2].

Analysis of the predicted MHC binding of peptides comprising mutations among proteins documented in the TCGA shows no statistical difference in overall predicted binding affinity between mutant and wildtype homolog. However, for TCEM I there is a significant impact when the mutant amino acid lies in positions 2 or 9 of a 9 mer. Overall, based on analysis of proteins with mutations recorded in TCGA, the MHC I binding affinity of the peptides containing the T cell exposed motif which become mutated is very low; about 22 μM, which is more than 40× lower than the 500 nM that is the consensus T cell stimulatory level. This indicates that such peptides are overall not highly likely to naturally elicit an effective and sustained cytotoxic T cell response and memory.

In one embodiment, the present invention enables the design of peptides presenting the T cell exposed motif of interest with a range of MHC binding affinities, allowing for selection of very high affinity binders or intermediate binding affinity to the alleles of a particular patient with the goal of stimulating and effective cytotoxic response. This is achieved by modification of amino acids in the groove exposed motifs, without changing the T cell exposed motifs to achieve a desired binding for one or more of a subject's MHC alleles.

### Frequency Characteristics of Peptides Generated by Mutations in Cancer

Comparison of the frequency distribution of the T cell exposed motifs in peptides comprising mutations (for TCEM I cognate for MHC I molecules), among those documented in the TCGA, reveals that those comprising mutated amino acids are motifs that occur less commonly in the human proteome than their wildtype homologues. Overall the mutant peptides are biased towards those that are rare or even completely absent in the human proteome; the comparator here being all T cell exposed motif in all peptides of all isoforms of human proteins, approximately 88,000 proteins. The mutational event that inserts a new amino acid in the T cell exposed motif consistently produces T cell exposed motif that are much rarer as compared to the wildtype T cell exposed motif. (See, e.g., PCT US2020/037206, incorporated herein by reference in its entirety). It was also noted that when the frequency category of the T cell exposed motif comprising mutated acids in tumors are compared to the frequency of occurrence in the human immunoglobulinome, they correspond on average to the immunoglobulin frequency category FC20; indicating that on average the T cell exposed motif amino acid motifs would be found in 1 in 2²⁰ immunoglobulin variable regions (less than 1 in a million B cell clonal lines). This is 1000-fold below the mean frequency in immunoglobulin variable regions; another indicator that tumor T cell exposed motif are uncommon and that there may be a low frequency of cognate T cells.

### Cross Presentation of MHC I and II Binding Peptides

While the primary focus is on stimulating a cytotoxic T cell response, driven by CD8+ T cells, such a response is enhanced and helped by the simultaneous stimulation of a CD4+ T helper response. This may be particularly important to the development of a population of memory T cells which can ensure ongoing surveillance and elimination of cancer cells. In some instances, a naturally occurring T helper response may be driven from the native mutated protein. In the present invention we also describe how a tumor specific T helper response can be stimulated by peptides designed to have a high binding affinity to the patient's MHC II alleles and to target T cell exposed motifs which comprise the mutated amino acid. Therefore, in one embodiment the invention provides for designing 15mer peptides by maintaining the TCEM II and varying the flanking sequences.

### Maximizing Targeting of Mutations and Stimulation of Cytotoxic T Cell Responses in a Personalized Vaccine

In some embodiments, to optimize the second phase personalized vaccination and similarly to maximize targeting of the selected common mutations of the first phase of vaccination, the present invention provides methods to maximize the utilization of available tumor specific antigens to generate effective cytotoxic T cell response that can bring about elimination of the tumor cells. This is achieved by identifying the T cell exposed motif containing the mutant amino acids and generating an array of peptides which combine these T cell exposed motifs with an array of different flanking amino acids of varying predicted binding affinity to enable selection of appropriate high binding peptides (See. e.g., PCT US2020/037206, which is incorporated by reference herein in its entirety).

In the case of TCEM I located in a 9-mer comprising 5 exposed amino acids flanked by 4 groove exposed amino acids, for each T cell exposed motif there is a maximum of 20⁴ or 160,000 possible variant amino acid combinations in the groove exposed position. In some embodiments, an array of 1000 peptides is created by random amino acid substitution in the groove exposed positions, in other embodiments an array of 10,000 peptides is likewise created, and in further embodiments a 50,000 peptide array is created. In the case of TCEM II to create peptides binding differentially to MHC II, we consider a 15 mer in which exposed positions 2, 3, 5, 7, 8 or −1, 3, 5, 7, 8 are kept constant, as all other amino acids in the peptide that are presumed to be involved in the binding affinity are changed by random substitution to create arrays of 1000, 5,000 or 10,000 peptides. In both cases the array sizes cited here are examples that are considered non limiting.

In each case, both MHC I and MHC II, the TCEM is maintained identical to that in the mutated peptides in the native mutated protein and all TCEM which comprise each mutated amino acid are selected as the basis for generation of binding variants.

In further steps embodied in this invention, the initial array of peptides generated by amino acid substitution is then filtered to remove any duplicate peptides, and in some preferred embodiments peptides predicted to be of low solubility are removed by assigning a score to the polarity of their constituent amino acids. The peptides are then selected to be suitable for the specific patient and his/her combination of MHC I and MHC II alleles. In preferred embodiments all alleles are typed, including MHC I A, MHC I B, MHC I C, and MHC II DRB, DP and DQ loci. In one embodiment, the predicted affinity of the peptides in the native mutant protein is reviewed to determine the probability that a particular peptide would be bound by one or more of the patient's MHC alleles, albeit with a low affinity, and hence presented for T cell recognition. As the goal is to stimulate or “train” T cells to target the specific mutated T cell exposed motifs (TCEM) in the tumor, these must be exposed to T cell recognition to enable targeting of tumor cells. In one embodiment we identify each of the TCEM-allele combinations in each native mutant protein which binds with an affinity greater than the mean for the comprising protein. Such TCEM are targetable by T cells which are also specific to that MHC allele histotope. TCEM-allele combinations which have a predicted binding affinity above the mean are set aside as unlikely to ever be presented. For this subset of “presentable” TCEM-allele combinations, we then assess the array of randomly generated peptides, filtered for binding and solubility, and identify a peptide for each TCEM-allele combination with a desired predicted binding affinity. In some embodiments, the peptide with maximum predicted binding affinity for each allele may be chosen. This may be a peptide that binds at 2.5 or 3 or more standard deviation units below the mean for peptides in the protein (i.e., higher affinity). Such a high binding peptide would be comparable to those detected as part of the presentome by mass spectroscopy and equivalent to approximately <20 nM to 100 nM, depending on the protein context. In preferred embodiments however, peptides are chosen with high, but not excessive predicted binding affinity, keeping in mind the probability that this may be more likely to stimulate an effective cytotoxic response and memory and mitigate against T cell exhaustion. Such a binding affinity may be from 1-2 standard deviation units below the mean for peptides in the protein, typically equivalent to 100-500 nM. Overall, the invention embodies the ability to select for a desired binding affinity and can be considered “tunable” to that selected binding affinity for each patient allele.

In some embodiments the novel peptides are used in vitro to stimulate dendritic cells or T cells. In some embodiments such cells are of autologous source, in yet other embodiments they are obtained from allele-matched donors. Stimulated cells are then administered to the cancer patient to passively provide an active T cell population or to provide dendritic cells presenting the TCEM of interest which can stimulate T cells in the patient. In yet other embodiments the peptides are used as components of a peptide vaccine. In yet other embodiments the peptides are applied as a fusion with antibody sequences. In further embodiments the peptides may be encoded in RNA or DNA for administration.

In some embodiments, the frequency classification of the TCEM in the human proteome is noted. In further embodiments the frequency classification of the TCEM in the human immunoglobulinome is noted. In both cases this is achieved by reference to a precomputed reference database comprising over 88,000 human proteins including multiple isoforms and over 35 million unique human immunoglobulin variable regions. Based on this, in some embodiments peptides comprising rare TCEM are identified for priority use.

In desired embodiments, therefore, the process described above yields a unique array of peptides for a particular patient, enabling stimulation of T cells targeting the maximum possible TCEM specific to that patient's tumor-specific mutations and mutated proteins, by presentation of peptides of selected binding affinity in each of the known alleles the patient carries, and the peptides further selected to be soluble. This is a panel of peptides which can then be deployed to stimulate T cells in vivo and in vitro by application in a number of different formats.

TCEMs comprise 5 amino acids, or 20⁵=3.2 million possible configurations. T cell receptor polyspecificity is well recognized [61]. Any neoantigen carries with it the risk of generating an off-target T cell targeting of a self-protein with potential adverse consequences, which may be magnified by immunomodulatory interventions such as checkpoint inhibitors. Prior developers of neoantigen vaccines have not addressed this aspect. In a further embodiment of the present invention therefore, TCEM are identified which comprise mutated amino acids and which are bound and presented in the patient's alleles, and are therefore identified as candidates for targeting with T cells stimulated by highly bound peptides. The stimulation of T cells targeting these peptides, when enhanced by high binding affinity neoantigens and potentially further boosted by a checkpoint inhibitor blockade could potentially give rise to self-protein targeting. In one embodiment, therefore, a “call list” of such TCEM is cross-correlated with the reference data set of the human proteome to identify all human proteins carrying the TCEM. These proteins are reviewed to determine the predicted binding affinity of the peptide in which the TCEM occurs for each of the patient's known alleles. If the human proteome carries that TCEM and the patient alleles would bind the contextual peptide at a moderate or high affinity (which may be considered to be an affinity at less than 1 standard deviation below the mean for the comprising protein, although this range is not considered limiting) then the protein carrying the TCEM is added to an advisory list. In preferred embodiments the protein is identified by its Uniprot identifier or identifiers linking it to other reference databases. In preferred embodiments the advisory list is reviewed to further identify proteins where deficiencies or blockades are associated with known pathologies, and to identify proteins which are of critical function and non-redundant. Such proteins may not be suitable for inclusion in a neoantigen vaccine and may be added to a caution list. However, the advisory and caution lists only identify potential sources of adverse reactions and must be weighed against the progression and severity of the cancer. Given the degree of inherent polyspecificity, the advisory list is typically quite extensive. Many proteins are shielded by anatomic or cellular location, some may be considered redundant, or may be considered an acceptable tradeoff to overcoming cancer. However, this embodiment allows an informed decision to be made regarding possible adverse effects in neoantigen selection.

### Design of Personalized Neoantigen Vaccines

In a preferred embodiment the present invention allows the design of a personalized immunotherapeutic intervention designed for each cancer patient based on their HLA alleles and particular set of mutations for application in the second vaccination. In some applications of this embodiment the mutations are unique to one patient. This intervention becomes feasible as soon as sequencing of a tumor biopsy and HLA typing is available and can be applied as a second round of vaccination as described herein. In some embodiments the process of sequencing a biopsy may be repeated several times in the course of treatment and the selection of peptides updated based on detection of new mutations. In some preferred embodiments the invention provides an immunotherapy solution for patients who have few proteins with known mutations, for example, but not limited to, glioblastoma patients, who would otherwise be limited to only one neoantigen per protein and possibly no neoantigens with appropriate HLA binding. The preferred embodiment of the present invention is to provide the maximum number of T cell stimulating peptides which will result in targeting of every possible TCEM in which the mutant amino acid occurs and by utilizing every possible HLA. In a further embodiment of the invention the peptides are down-selected to those which will target TCEM presented in vivo and those which are less likely to cause adverse targeting of other human proteins. In an extension of this preferred embodiment, the selected stimulatory peptides may be grouped to provide a series of vaccinations or treatments which allow the utilization of all available alleles the patient carries, while not causing competition for peptide presentation in any one group of peptides.

In some embodiments the selected peptides are applied to dendritic cells in vitro which are then administered to the patient to stimulate T cells. In yet other embodiments the selected peptides are applied in vitro to stimulate a population of T cells which are administered to the patient. In yet other embodiments the peptides, or nucleic acids encoding them are administered directly to the patient in one or more groups spaced over time.

### Utilization of Designed Peptides

In some embodiments the present invention will yield an array of many peptides suitable for enhancing the CD8+ response of a particular patient to his/her mutated tumor proteins and a list of many peptides suitable for enhancing a CD4+ helper response to these proteins. In some particular embodiments the number of peptides designed to bind MHC and stimulate T cells in a particular patient may be up to 5, in others it is about 20, in yet others it is over 100 and in yet others over 200 peptides. In some embodiments the peptide array will include those which bind to 1 allele, 2 alleles or up to 6 MHC I alleles and others which bind 1, 2 or up to 6 MHC II alleles. In order to optimize the application of the peptides and maximize the use of binding alleles while minimizing competition for binding at any single administration, a further embodiment of the present invention is to prioritize and group the peptides for sequential administration. In a preferred embodiment the peptides may be grouped into subgroups of about 5, in other embodiments subgroups of about 10 are preferred, and in yet other embodiments subgroups of about 20 are preferred and in further embodiments larger groups are preferred. The subgroups may combine both MHC I and MHC II binding peptides. Some peptides may be repeated in several subgroups. In some embodiments where vaccination regimens comprise sequential administration of a subset of selected peptides, each peptide administration may be followed by check point inhibitor treatment. In some embodiments, consideration is given to whether particular TCEM encompassed in the peptides in each group are rare or common TCEM in the human proteome or immunoglobulinome. In some preferred embodiments priority is given to inclusion of peptides that comprise rare TCEM. In each instance where a peptide is mentioned above, this may also refer to the application of a nucleic acid encoding the peptide. In preferred embodiments peptides that have TCEM matches in certain human proteins are excluded from consideration, where stimulating a T cell response which may target the human proteins may result in an adverse effect. In yet another embodiment, where transcription levels of the mutated proteins in a tumor are known, peptides may be prioritized based on their transcription level to increase the chance of successful targeting of tumor cells.

### Many Delivery Formations

Many delivery formulations are suitable for administration of both the first and second rounds of vaccinations described herein. In some embodiments the delivery formulation adopted may be the same for both first and second rounds of vaccinations. In other embodiments different delivery formulations may be chosen for tumor associated antigens, for epitopes targeting common mutations of the clinical presentation, and yet different formulations and delivery routes chosen for the personalized neoepitopes of the second round of vaccination

The suitable delivery formulations for neoepitope and tumor associated antigen vaccines, include but are not limited to peptide vaccines, antibody-antigen fusion proteins, DNA or RNA encoding antigens, and particulate vaccines. Neoantigens have been administered directly to subjects or have served to prime dendritic cells or stimulate T cells in vitro for administration of such cells to the subject. The dendritic cells or T cells have included those of autologous or of donor origin. Any of these delivery formulations may be used for delivery of peptides designed by the present invention.

In some embodiments of the present invention the epitope peptides, or their encoding nucleic acids, may be administered parenterally. In yet other embodiments the peptides or their encoding nucleic acids may be delivered intradermally or subcutaneously. In some embodiments intradermal administration may be achieved by needle injection. In preferred embodiments intradermal administration may be provided by micro needle patch or array. In yet further embodiments the microneedle patch or array may deliver multiple different peptides or encoding nucleic acids thereof. In yet other embodiments some doses of each round of vaccination may be delivered orally.

In some embodiments of the present invention the peptides comprising the selected epitopes may be delivered as a nucleic acid, in some embodiments the nucleic acid may be RNA, in others DNA. In further embodiments the peptides may be delivered by a virus vector, which may comprise elements of a retroviral vector, a poxvirus, an adenovirus, or other virus vector delivery system. In further embodiments the delivery of nucleic acid or peptide may be accomplished by a lipid drug delivery system comprising, but not limited to, one of the following: nanoparticles, emulsions, self-emulsifying drug delivery systems, nanocapsules and liposomes, wherein molecules of a drug active product is encased or partially encases in lipid.

In some embodiments the selected peptides may be administered as a fusion to a moiety which favors formation of nanoparticles. Examples of such moieties include but are not limited to leucine multimers (polyleucine), unnatural hydrophobic amino acids, or liposomes. The peptide of interest may be attached to its fusion partner by a linker. In some instances, the linker is cleavable. The cleavable linker may be one or more lysine or arginine residues, or a cathepsin cleavable linker.

In some embodiments the selected peptides or their encoding nucleic acids may be delivered with an adjuvant. Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

In some embodiments epitope peptides stimulating a response to tumor associated antigens have been selected to allow binding to an array of different HLA alleles and such vaccines may be administered without determination of the HLA alleles of the subject. However, in a preferred embodiment the HLAs of the subject are determined prior to administration of the first round of vaccination. For the second round of vaccination, having knowledge of the patient's HLA alleles is a prerequisite to designing a bespoke peptide vaccine. Several approaches to HLA typing may be employed, including PCR, and such testing is widely available. The HLA can be derived from the whole exome sequence at the same time as analysis of the tumor mutations in the biopsy.

The present invention provides an immunotherapeutic strategy to maximize the immune response to a tumor by combining vaccinations comprising epitopes derived from tumor associated antigens, epitopes that are arise from mutations commonly found in cancers of a particular clinical presentation, and personalized combinations of neoepitopes designed to address the particular mutational landscape of one individual subject, based sequencing the exome of a tumor biopsy from that person. In preferred embodiments, this is a staged approach with the first round of vaccination occurring prior to surgical intervention and a second round following surgical intervention and biopsy. However, in situations where the tumor is not respectable due to its location, and in instances where the personal tumor mutational landscape can be determined by sequencing tissues accessible without surgery the two phases may proceed without a surgical intervention.

### EXAMPLES

### Example 1: Epitope Peptides in VEGFR

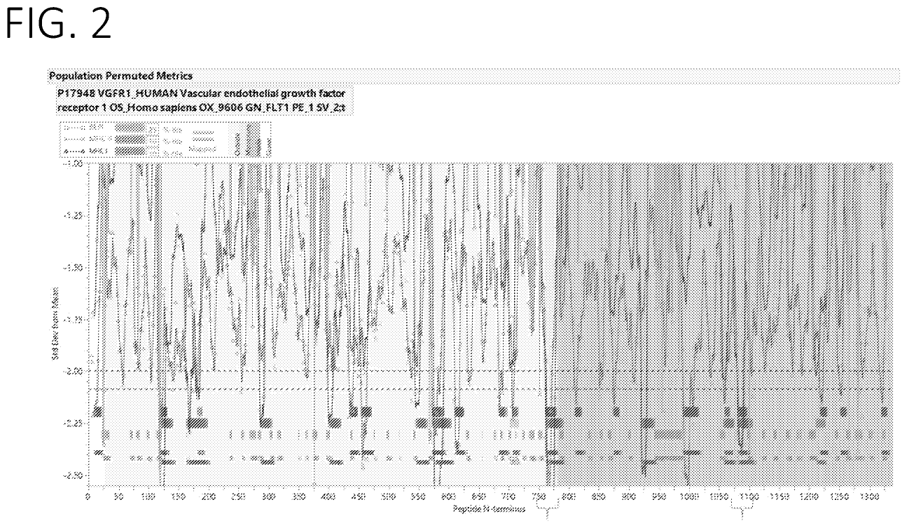

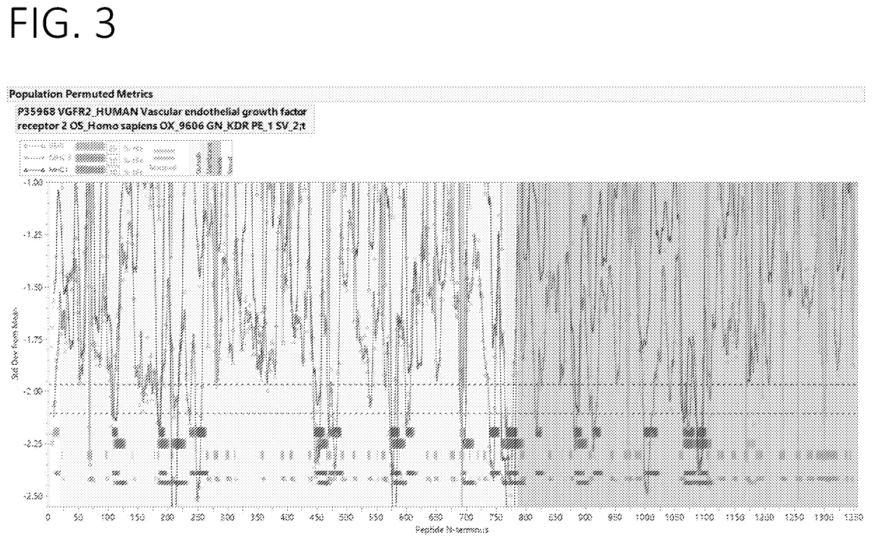

Vascular endothelial growth factor receptor 1 (VEGFR1), also known as FLT1, is characterized by the sequence P17948 (uniprot.org) and also occurs in at least 8 other isoforms produced by alternative splicing. In selecting an epitope peptide therefore, it is important to identify sequences which are conserved. VEGFR1 is a tyrosine-protein kinase that acts as a cell-surface receptor for VEGFA, VEGFB and PGF, and plays an essential role in the development of embryonic vasculature, the regulation of angiogenesis, cell survival, cell migration, macrophage function, chemotaxis, and cancer cell invasion. Vascular endothelial growth factor receptor 2 (VEGFR2), also known as KDR, is similarly a tyrosine-protein kinase, but which acts as a cell-surface receptor for VEGFA, VEGFC and VEGFD. FIGS. 2 and 3 provide an overview of the highest MHC I and MHC II binding region in the protein. Tables 1 and 2 provide peptide sequences selected based on their predicted affinity to 31 MHC I A and 31 MHC I B and 8 MHC I C alleles and 23 MHC II DRB alleles.

TABLE 1
Selected VEGFR1 and VEGFR2 MHC I epitope peptides and their binding

| | | SEQ | | SEQ | | | | | | | | | | | | | | | | |
|---|---|---|---|---|---|---|---|---|---|---|---|---|---|---|---|---|---|---|---|---|
| cura- | | ID | | ID | A | A | A | A | A | A | A | A | A | A | A | A | A | A | A | |
| tion | | NO: | 9-mer | NO: | 0101 | 0201 | 0202 | 0203 | 0206 | 0211 | 0212 | 0216 | 0217 | 0219 | 0250 | 0301 | 0801 | 1101 | 2301 | |
| VGFR1 | VAATLFWLLL | 1 | VAATLFWLL | 5 | -1.75 | -2.32 | -1.57 | -0.95 | -1.93 | -1.04 | 0.40 | -0.15 | -1.30 | -0.76 | -0.08 | -1.07 | -1.20 | -1.28 | -1.28 | |
| VGFR1 | | | AATLFWLLL | 6 | -1.31 | -1.79 | -1.21 | -0.93 | -1.29 | -0.48 | 0.97 | -0.15 | -0.61 | 0.05 | 0.02 | -1.08 | -1.54 | -1.46 | -1.10 | |
| VGFR1 | VLLWEIFSLG | 2 | VLLWEIFSL | 7 | 0.38 | -3.59 | -2.03 | -1.70 | -1.92 | -1.58 | -1.65 | -1.46 | -0.61 | -2.13 | -0.96 | -0.44 | -3.29 | -0.33 | -2.55 | |
| VGFR1 | | | LLWEIFSLG | 8 | -0.52 | -3.16 | -2.39 | -2.50 | -3.43 | -2.43 | -2.31 | -2.29 | -0.19 | -2.43 | -1.54 | -0.52 | -1.33 | 0.08 | -0.68 | |
| VGFR2 | VIAMFFWLLL | 3 | VIAMFFWLL | 9 | -0.80 | -2.78 | -2.21 | -1.56 | -1.94 | -1.66 | -2.04 | -1.20 | -1.36 | -1.72 | -1.20 | -1.23 | -1.37 | -1.16 | -2.27 | |
| VGFR2 | | | IAMFFWLLL | 10 | -0.96 | -2.48 | -1.52 | -1.29 | -2.32 | -0.63 | 0.41 | 0.26 | -1.57 | -0.42 | -0.17 | -1.10 | -2.67 | -1.43 | -2.07 | |
| VGFR2 | DVWSFGVLLW | 4 | DVWSFGVL | 11 | -0.20 | -2.19 | -1.64 | -1.51 | -1.25 | -1.92 | -1.51 | -0.24 | -1.67 | -1.18 | -0.07 | -1.66 | -0.96 | -1.01 | -1.45 | |
| | | | L | | | | | | | | | | | | | | | | | |
| VGFR2 | | | VWSFGVLL | 12 | -0.69 | 2.72 | -1.46 | -0.62 | -0.48 | 0.15 | -0.14 | 0.01 | -0.28 | -0.13 | -0.33 | -0.87 | -0.27 | -1.31 | -2.77 | |
| | | | W | | | | | | | | | | | | | | | | | |
| cura- | | | | | A | A | A | A | A | A | A | A | A | A | A | A | A | A | A | A |
| tion | | | 9-mer | | 2402 | 2403 | 2501 | 2601 | 2602 | 2603 | 2902 | 3001 | 3002 | 3101 | 3201 | 3301 | 6801 | 6802 | 6901 | 8001 |
| VGFR1 | VAATLFWLLL | 1 | VAATLFWILL | 5 | -1.89 | -0.85 | -0.62 | -0.27 | -2.39 | -0.78 | -1.36 | 0.64 | 0.98 | -0.36 | -0.94 | -0.30 | -0.24 | -1.67 | -2.30 | -1.41 |
| VGFR1 | | | AATLFWLLL | 6 | -1.40 | -1.18 | -0.55 | -0.61 | -2.24 | -0.62 | -0.92 | -0.95 | 0.85 | -0.54 | -1.18 | -0.44 | -0.10 | -1.36 | -1.46 | -1.30 |
| VGFR1 | VLLWEIFSLG | 2 | VLLWEIFSL | 7 | -2.21 | -2.58 | -0.16 | 0.14 | -1.56 | -0.42 | -1.05 | 0.34 | 1.10 | -0.43 | -2.22 | -0.30 | 0.00 | -0.79 | -1.82 | -0.94 |
| VGFR1 | | | LLWEIFSLG | 8 | -0.40 | 0.39 | 0.13 | -0.95 | -0.14 | -0.30 | -0.47 | -1.67 | -0.02 | -0.78 | -0.10 | -0.29 | 0.45 | -1.26 | -1.84 | 0.00 |
| VGFR2 | VIAMFFWLLL | 3 | VIAMFFWLL | 9 | -2.18 | -2.07 | 0.12 | -1.17 | -1.43 | -0.28 | -1.46 | 0.29 | 1.97 | -0.86 | -1.66 | -0.67 | -0.51 | -1.68 | -1.92 | -1.64 |
| VGFR2 | | | IAMFFWLLL | 10 | -1.64 | -1.14 | -0.79 | -0.86 | -2.38 | -0.73 | -1.37 | -0.94 | -0.02 | -1.00 | -1.18 | -0.93 | -0.81 | -1.54 | -2.03 | -1.95 |
| VGFR2 | DVWSFGVLLW | 4 | DVWSFGVL | 11 | -1.27 | -1.41 | -2.19 | -1.98 | -0.67 | -0.33 | -1.17 | -1.74 | 0.25 | -0.65 | -2.76 | -0.97 | -0.52 | -2.19 | -2.38 | -1.87 |
| | | | L | | | | | | | | | | | | | | | | | |
| VGFR2 | | | VWSFGVLL | 12 | -2.95 | -3.69 | -0.37 | 0.52 | -0.43 | 1.25 | -2.48 | 1.39 | 0.69 | -0.24 | -0.81 | -1.22 | -1.27 | 1.10 | 0.83 | -2.22 |
| | | | W | | | | | | | | | | | | | | | | | |
| cura- | | | | | B | B | B | B | B | B | B | B | B | B | B | B | B | B | B | |
| tion | | | 9-mer | | 0702 | 0801 | 0802 | 0803 | 1501 | 1502 | 1503 | 1509 | 1517 | 1542 | 1801 | 2703 | 2705 | 3501 | 3801 | |
| VGFR1 | VAATLFWLLL | 1 | VAATLFWLL | 5 | -1.31 | -1.32 | -0.22 | 0.05 | 0.04 | 0.13 | -0.32 | 0.31 | -2.07 | 0.68 | -0.39 | 0.54 | 0.27 | -1.73 | 0.19 | |
| VGFR1 | | | AATLFWLLL | 6 | -1.33 | -2.11 | 0.46 | -1.46 | 0.19 | -0.12 | 0.70 | 1.50 | -2.06 | 1.30 | -0.20 | 0.11 | 0.03 | -1.11 | 0.23 | |
| VGFR1 | VLLWEIFSLG | 2 | VLLWEIFSL | 7 | -1.19 | -2.77 | -0.68 | 0.30 | -1.05 | 1.05 | 0.14 | -1.19 | -1.27 | 0.38 | -1.27 | -0.59 | 0.31 | -1.11 | 0.96 | |
| VGFR1 | | | LLWEIFSLG | 8 | -0.18 | -1.65 | 0.76 | -1.02 | -1.25 | -0.67 | -0.50 | -0.23 | 0.26 | -0.40 | 0.27 | -0.53 | -0.08 | -0.53 | -0.27 | |
| VGFR2 | VIAMFFWLLL | 3 | VIAMFFWLL | 9 | -0.65 | -2.10 | -0.99 | -1.02 | 0.23 | -0.34 | 0.47 | 0.15 | -0.74 | 1.07 | -0.98 | -0.64 | 0.20 | -0.80 | -0.29 | |
| VGFR2 | | | IAMFFWLLL | 10 | -1.59 | -2.59 | 0.30 | -0.89 | -0.55 | -0.73 | -0.01 | 0.23 | -2.62 | 0.85 | -0.76 | -0.03 | -0.21 | -2.36 | -0.39 | |
| VGFR2 | DVWSFGVLLW | 4 | DVWSFGVL | 11 | -1.72 | -0.90 | 0.49 | -0.42 | -1.36 | -1.49 | -0.32 | -0.80 | -2.50 | 0.58 | -0.65 | -0.48 | -0.36 | -0.49 | -0.70 | |
| | | | L | | | | | | | | | | | | | | | | | |
| VGFR2 | | | VWSFGVLL | 12 | 0.39 | 0.16 | -0.89 | 0.69 | 0.01 | -0.06 | -2.60 | 1.02 | -0.35 | 1.63 | -1.38 | -0.30 | 0.40 | -1.57 | -0.80 | |
| | | | W | | | | | | | | | | | | | | | | | |
| | | SEQ | | SEQ | | | | | | | | | | | | | | | | |
| cura- | | ID | | ID | B | B | B | B | B | B | B | B | B | B | B | B | B | B | | |
| tion | | NO.: | 9-mer | NO.: | 3901 | 4001 | 4002 | 4402 | 4403 | 4501 | 4506 | 4601 | 4801 | 5101 | 5301 | 5401 | 5701 | 5801 | | |

| VGFR1 | VAATLFWLLL | 1 | VAATLFWLL | 5 | -1.60 | -0.37 | -1.28 | -0.88 | -1.10 | -0.84 | 0.58 | 0.84 | -1.57 | -2.66 | -2.17 | -0.57 | -2.65 | -2.96 |
|---|---|---|---|---|---|---|---|---|---|---|---|---|---|---|---|---|---|---|
| VGFR1 | | | AATLFWLLL | 6 | -0.37 | 0.01 | -1.00 | -0.60 | -0.81 | -0.20 | 0.73 | 1.03 | -1.10 | -2.38 | -1.11 | -0.58 | -2.51 | -2.16 |
| VGFR1 | VLLWEIFSLG | 2 | VLLWEIFSL | 7 | -0.72 | 0.01 | -0.99 | -0.25 | -0.86 | -0.41 | 0.24 | -0.33 | 0.61 | -1.24 | -0.99 | -0.10 | -0.59 | -0.48 |
| VGFR1 | | | LLWEIFSLG | 8 | 0.15 | -0.03 | -0.66 | 0.69 | -0.27 | -0.37 | -0.34 | -0.89 | -0.48 | -1.26 | 0.01 | -2.14 | 0.17 | 0.55 |
| VGFR2 | VIAMFFWLLL | 3 | VIAMFFWLL | 9 | -1.34 | -0.15 | -1.03 | -0.52 | -1.43 | -1.22 | -0.43 | 0.57 | -0.86 | -1.02 | -1.36 | -0.42 | -0.20 | -0.80 |
| VGFR2 | | | IAMFFWLLL | 10 | -2.10 | -1.43 | -1.65 | -0.73 | -1.31 | -0.46 | 0.62 | -0.48 | -2.88 | -3.69 | -1.89 | -2.26 | -3.30 | -2.59 |
| VGFR2 | DVWSFGVLLW | 4 | DVWSFGVLL | 11 | -2.04 | -0.17 | -1.13 | 0.31 | -1.62 | -1.46 | 0.29 | -0.90 | -4.41 | -0.48 | -1.15 | 0.17 | -1.96 | -1.44 |
| VGFR2 | | | VWSFGVLLW | 12 | 0.07 | 0.26 | -0.61 | -0.40 | -1.61 | 0.12 | 0.90 | 0.10 | 0.31 | -0.63 | -1.96 | 1.11 | -1.12 | -1.26 |
| cura- | | | | | C | C | C | C | C | C | C | C | | | | | | |
| tion | | | 9-mer | | 0303 | 0401 | 0501 | 0602 | 0702 | 1203 | 14002 | 1502 | | | | | | |
| VGFR1 | VAATLFWLLL | 1 | VAATLFWLL | 5 | -1.07 | -1.27 | -0.21 | 1.93 | 2.86 | 0.79 | 1.66 | -1.31 | | | | | | |
| VGFR1 | | | AATLFWLLL | 6 | -0.33 | 0.16 | -0.35 | 2.16 | 2.29 | 0.61 | 3.06 | -0.92 | | | | | | |
| VGFR1 | VLLWEIFSLG | 2 | VLLWEIFSL | 7 | 0.23 | -0.03 | 1.54 | 2.02 | -0.15 | 1.83 | -0.28 | 1.62 | | | | | | |
| VGFR1 | | | LLWEIFSLG | 8 | 0.59 | -0.94 | 1.05 | 0.69 | 1.02 | -2.50 | -2.06 | -0.05 | | | | | | |
| VGFR2 | VIAMFFWLLL | 3 | VIAMFFWLL | 9 | 0.44 | 0.41 | 0.27 | 2.05 | 1.52 | 1.88 | -0.11 | 0.74 | | | | | | |
| VGFR2 | | | IAMFFWLLL | 10 | -0.54 | -0.68 | 0.24 | 1.89 | 2.57 | 0.69 | 1.17 | -1.47 | | | | | | |
| VGFR2 | DVWSFGVLLW | 4 | DVWSFGVLL | 11 | 0.90 | -1.51 | 0.70 | 1.43 | 1.03 | -0.44 | -0.31 | -2.41 | | | | | | |
| VGFR2 | | | VWSFGVLLW | 12 | 0.36 | -1.17 | 0.97 | 2.39 | -0.08 | 2.11 | -0.66 | 3.31 | | | | | | |

NOTE
Binding is shown in standard deviation units comparing all peptides within the protein. While actual affinity varies between proteins a value of -1 SD equates to 100-200 nanomolar

TABLE 2
Selected VEGFR1 and VEGFR2 MHC II epitope peptides and their binding

| | | SEQ | | SEQ | | | | | | | | | | | | |
|---|---|---|---|---|---|---|---|---|---|---|---|---|---|---|---|---|
| | | ID | | ID | DRB1 | DRB1 | DRB1 | DRB1 | DRB1 | DRB1 | DRB1 | DRB1 | DRB1 | DRB1 | DRB1 | DRB1 |
| curation | | NO.: | peptide | NO.: | 0101 | 0301 | 0401 | 0404 | 0405 | 0701 | 0801 | 0802 | 0901 | 1001 | 1101 | 1201 |
| VGFR1 | ATLFWLLLTLFI | | ATLFWLLLTL | 17 | -1.31 | -2.35 | -1.87 | -2.80 | -3.50 | -1.31 | -3.60 | -2.86 | -2.05 | -2.57 | -2.90 | -3.23 |
| | RKMKR | | FIRKM | | | | | | | | | | | | | |
| VGFR1 | | 13 | TLFWLLLTLFI | 18 | -1.62 | -2.29 | -2.50 | -3.55 | -3.37 | -1.84 | -3.00 | -0.92 | -1.46 | -1.22 | -2.83 | -2.11 |
| | | | RKMK | | | | | | | | | | | | | |
| VGFR1 | | | LFWLLLTLFI | 19 | -1.41 | -2.45 | -1.77 | -2.82 | -2.76 | -2.61 | -3.15 | -2.12 | -2.10 | -1.38 | -2.94 | -2.71 |
| | | | RKMKR | | | | | | | | | | | | | |
| VGFR1 | KSDVWSYGVL | | KSDVWSYGV | 20 | -0.84 | -1.30 | -1.33 | -2.12 | -1.78 | -1.92 | -0.97 | -0.74 | -1.98 | -0.46 | -0.51 | -2.61 |
| | LWEIFSL | | LLWEIF | | | | | | | | | | | | | |
| VGFR1 | | 14 | SDVWSYGVL | 21 | -1.63 | -0.81 | -0.84 | -1.38 | -1.91 | -1.33 | -1.25 | -0.18 | -1.91 | -1.02 | -0.86 | -1.75 |
| | | | LWEIFS | | | | | | | | | | | | | |
| VGFR1 | | | DVWSYGVLL | 22 | -0.74 | 0.40 | -1.06 | -1.14 | -1.81 | -1.64 | -1.77 | -0.44 | -1.00 | -2.10 | -0.86 | -1.88 |
| | | | WEIFSL | | | | | | | | | | | | | |
| VGFR2 | GTAVIAMFFWL | | GTAVIAMFF | 23 | -0.37 | -0.66 | -1.11 | -1.95 | -1.61 | -0.61 | -2.40 | -1.95 | -1.73 | -2.05 | -0.94 | -2.89 |
| | LLVIIL | | WLLLVI | | | | | | | | | | | | | |
| VGFR2 | | 15 | TAVIAMFFWL | 24 | -0.47 | -0.79 | -1.07 | -2.78 | -2.50 | -1.62 | -2.79 | -2.51 | -1.19 | -1.54 | -1.78 | -2.94 |
| | | | LLVII | | | | | | | | | | | | | |
| VGFR2 | | | AVIAMFFWLL | 25 | -0.21 | -0.71 | -1.40 | -2.71 | -2.26 | -1.32 | -2.78 | -3.47 | -2.02 | -2.72 | -2.05 | -3.04 |
| | | | LVIIL | | | | | | | | | | | | | |
| VGFR2 | DVWSFGVLLW | | DVWSFGVLL | 26 | -0.58 | -0.02 | -1.30 | -1.56 | -1.91 | -1.68 | -1.85 | -0.93 | -1.23 | -2.19 | -0.99 | -1.93 |
| | EIFSLGA | | WEIFSL | | | | | | | | | | | | | |
| VGFR2 | | 16 | VWSFGVLLW | 27 | -1.21 | -0.05 | -1.52 | -2.17 | -0.99 | -1.23 | -2.28 | -0.42 | -0.84 | -0.22 | -1.41 | -1.62 |
| | | | EIFSLG | | | | | | | | | | | | | |
| VGFR2 | | | WSFGVLLWE | 28 | -0.61 | 0.05 | -0.90 | -2.13 | -1.69 | -1.33 | -2.18 | -2.51 | -1.89 | -2.13 | -1.82 | -2.17 |
| | | | IFSLGA | | | | | | | | | | | | | |

| | | | | | DRB1 | DRB1 | DRB1 | DRB1 | DRB1 | DRB3 | DRB3 | DRB3 | DRB4 | DRB4 | DRB5 |
|---|---|---|---|---|---|---|---|---|---|---|---|---|---|---|---|
| curation | | | peptide | | 1301 | 1302 | 1454 | 1501 | 1602 | 0101 | 0202 | 0301 | 0101 | 0103 | 0101 |
| VGFR1 | ATLFWLLLTLFI | | ATLFWLLLTL | 17 | -3.33 | -1.99 | -3.65 | -2.61 | -1.74 | -2.41 | -3.87 | -2.85 | -3.27 | -3.57 | -3.70 |
| | RKMKR | | FIRKM | | | | | | | | | | | | |
| VGFR1 | | 13 | TLFWLLLTLFI | 18 | -2.67 | -1.88 | -2.88 | -3.27 | 0.21 | -1.52 | -2.94 | -2.94 | -2.85 | -2.55 | -3.50 |
| | | | RKMK | | | | | | | | | | | | |
| VGFR1 | | | LFWLLLTLFI | 19 | -3.04 | -1.29 | -2.72 | -2.68 | -0.42 | -0.65 | -2.76 | -1.94 | -2.20 | -3.22 | -3.13 |
| | | | RKMKR | | | | | | | | | | | | |
| VGFR1 | KSDVWSYGVL | | KSDVWSYGV | 20 | -0.88 | -2.37 | -1.85 | -1.47 | -1.41 | -1.56 | -2.20 | -2.28 | -1.25 | -1.04 | -1.01 |
| | LWEIFSL | | LLWEIF | | | | | | | | | | | | |
| VGFR1 | | 14 | SDVWSYGVL | 21 | -1.50 | -0.66 | -2.04 | -1.96 | -1.28 | -1.67 | -2.16 | -1.54 | 1.62 | -1.07 | -0.77 |
| | | | LWEIFS | | | | | | | | | | | | |
| VGFR1 | | | DVWSYGVLL | 22 | -1.54 | -1.89 | -2.67 | -1.78 | 0.31 | -0.50 | -1.57 | -1.80 | -1.99 | -1.01 | -0.83 |
| VGFR2 | GTAVIAMFFWL | | GTAVIAMFF | 23 | -2.38 | -2.29 | -2.65 | -1.52 | -0.25 | -2.67 | -1.31 | -2.24 | -1.55 | -1.56 | -0.46 |
| | LLVIIL | | WLLLVI | | | | | | | | | | | | |
| VGFR2 | | 15 | TAVIAMFFWL | 24 | -2.67 | -1.69 | -2.91 | -1.21 | -0.07 | -3.04 | -1.59 | -3.06 | -2.64 | -2.49 | -0.64 |
| | | | LLVII | | | | | | | | | | | | |
| VGFR2 | | | AVIAMFFWLL | 25 | -2.76 | -1.46 | -3.27 | -1.03 | -0.78 | -2.90 | -1.94 | -2.63 | -3.41 | -2.13 | -0.89 |
| | | | LVIIL | | | | | | | | | | | | |
| VGFR2 | DVWSFGVLLW | | DVWSFGVLL | 26 | -1.54 | -2.15 | -2.47 | -1.92 | 0.11 | -0.69 | -1.72 | -2.00 | -2.03 | -1.19 | -0.90 |
| | EIFSLGA | | WEIFSL | | | | | | | | | | | | |
| VGFR2 | | 16 | VWSFGVLLW | 27 | -1.82 | -0.69 | -2.07 | -1.99 | 0.58 | -0.74 | -0.28 | -2.21 | -1.54 | -2.04 | -1.44 |
| VGFR2 | | | WSFGVLLWE | 28 | -1.57 | -0.67 | -2.15 | -0.91 | 0.37 | -0.70 | -0.96 | -0.69 | -1.72 | -2.14 | -1.03 |
| | | | IFSLGA | | | | | | | | | | | | |

NOTE
Binding is shown in standard deviation units comparing all peptides within the protein. While actual affinity varies between proteins a value of -1 SD equates to 100-200 nanomolar

### Example 2: Epitope Peptides in Vimentin

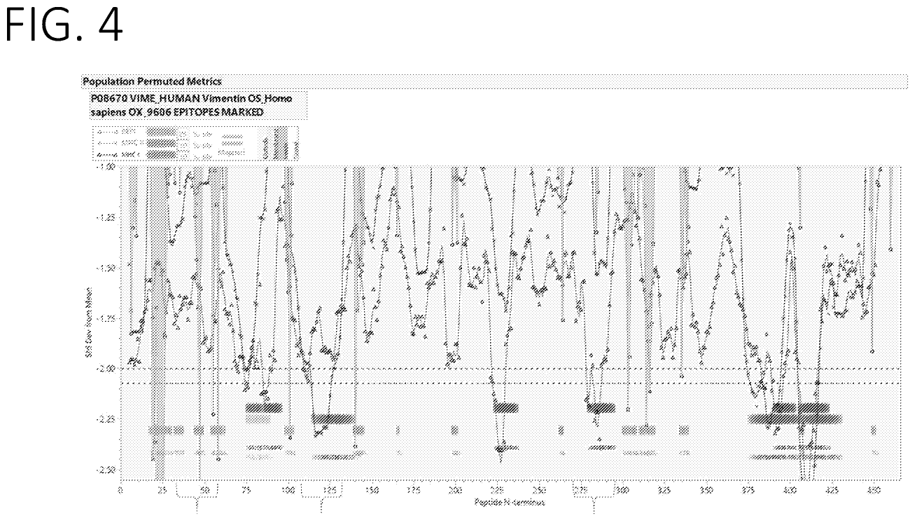

Vimentin is found in one dominant isoform represented by the sequence P08670 in Uniprot. Possible additional isoforms have been identified. FIG. 4 provides an overview of the high MHC II and MHC I binding regions of vimentin with locations of selected sequences identified. Tables 3 and 4 provide epitope peptides of vimentin which are predicted to bind multiple MHC I and MHC II alleles. A combination of two or more MHC I epitopes may be applied to provide broad coverage, or a peptide may be selected based on the known HLA alleles of the subject. In preferred embodiments an MHC I epitope peptide is administered contemporaneously with a MHC II peptide that overlaps it and also binds to the alleles of the individual.

TABLE 3
Selected vimentin MHC I epitope peptides and their binding

| | SEQ | | SEQ | | | | | | | | | | | | | | | | |
|---|---|---|---|---|---|---|---|---|---|---|---|---|---|---|---|---|---|---|---|
| | ID | | ID | A | A | A | A | A | A | A | A | A | A | A | A | A | A | A | A |
| peptide | NO.: | 9-mer | NO.: | 0101 | 0201 | 0202 | 0203 | 0206 | 0211 | 0212 | 0216 | 0217 | 0219 | 0250 | 0301 | 0801 | 1101 | 2301 | 2402 |
| SLYASSPGGV | 29 | SLYASSPGG | 41 | 1.51 | -2.41 | -1.17 | -2.05 | -1.09 | -2.58 | -2.88 | -2.39 | 0.94 | -2.56 | -1.00 | -0.40 | -0.67 | 0.30 | -0.67 | -0.02 |
| | | LYASSPGGV | 42 | 1.31 | -0.32 | -2.34 | -2.42 | -2.41 | -1.77 | -2.58 | -2.33 | 1.19 | -2.32 | -1.57 | 0.08 | -0.48 | 0.32 | -0.87 | -1.97 |
| YASSPGGVY | 30 | YASSPGGVY | 43 | -3.48 | -0.18 | -0.75 | -0.56 | 0.03 | 0.29 | 0.27 | 0.60 | -0.52 | -0.07 | 0.41 | -0.94 | 0.76 | -0.38 | 1.11 | 0.25 |
| SPGGVYATR | 31 | SPGGVYATR | 44 | -0.84 | -0.22 | 0.04 | 1.61 | -0.22 | 0.83 | 0.38 | -0.24 | 1.14 | -0.02 | 1.57 | -1.83 | 1.96 | -1.84 | 0.08 | 0.39 |
| GVYATRSSAV | 32 | GVYATRSSA | 45 | 1.44 | 0.32 | -0.01 | -1.19 | -0.30 | -2.03 | -2.04 | -1.55 | -0.74 | -2.29 | -0.80 | -0.01 | -0.85 | -0.02 | 0.60 | 2.01 |
| | | VYATRSSAV | 46 | 1.76 | -0.89 | -1.93 | -2.14 | -1.89 | -1.04 | -1.71 | -1.03 | 1.26 | -1.60 | -1.48 | -0.01 | -1.07 | 0.34 | -0.27 | -1.58 |
| ELQELNDRFA | 33 | ELQELNDRF | 47 | 0.55 | -0.64 | -0.99 | 0.27 | 1.40 | -0.47 | -0.34 | -0.28 | -1.83 | 0.20 | -0.21 | 0.81 | -0.38 | 1.71 | -2.60 | -1.81 |
| | | LQELNDRFA | 48 | -1.44 | 0.57 | -0.67 | -0.68 | -0.32 | -0.23 | 0.06 | -0.60 | 0.52 | -0.50 | -1.91 | -0.10 | -0.32 | 0.62 | 0.45 | 0.17 |
| ELNDRFANY | 34 | ELNDRFANY | 49 | -2.93 | -0.29 | -0.18 | -0.62 | 0.64 | -0.04 | 0.77 | -0.09 | -0.69 | 0.06 | 0.60 | -1.47 | -0.68 | -0.24 | -0.67 | -0.32 |
| RFANYIDKVR | 35 | IRFANYIDKV | 50 | 2.59 | -2.70 | -2.38 | -2.66 | -2.45 | -2.20 | -2.43 | -2.04 | -1.50 | -2.61 | -2.34 | -0.25 | -2.09 | 0.31 | -1.84 | -2.27 |
| | | FANYIDKVR | 51 | -1.54 | -0.38 | 0.76 | 0.74 | 1.30 | 1.09 | 0.47 | 1.35 | -1.66 | 0.45 | 1.59 | 0.55 | 0.82 | -1.15 | 1.14 | 0.11 |
| NYIDKVRFL | 36 | NYIDKVRFL | 52 | 0.10 | -1.33 | -2.13 | -1.80 | -1.79 | -0.58 | -0.95 | -0.19 | -1.00 | -1.19 | -2.18 | 0.13 | -1.15 | 0.47 | -2.82 | -2.54 |
| NLQEAEEWYK | 37 | NLQEAEEWY | 53 | -2.56 | -0.49 | -0.61 | 0.00 | 1.01 | -0.31 | 0.32 | -0.28 | -2.21 | 0.07 | -0.59 | -0.83 | 0.28 | -0.51 | -0.99 | -0.32 |
| | | LQEAEEWYK | 54 | -0.95 | 0.79 | 0.56 | 0.60 | 0.81 | 1.08 | 1.09 | 0.49 | 0.17 | 0.98 | 0.00 | -1.70 | 0.42 | -2.45 | -0.73 | 0.31 |
| EAEEWYKSK | 38 | EAEEWYKSK | 55 | -1.45 | -0.03 | 0.68 | 0.21 | 1.95 | 1.06 | 2.61 | 1.12 | -1.27 | 0.68 | 1.33 | -1.09 | 0.94 | -2.21 | 0.33 | 1.41 |
| WYKSKFADLS | 39 | WYKSKFADL | 56 | 0.73 | -0.56 | -2.23 | -2.21 | -1.64 | -1.33 | -1.09 | -0.59 | -1.19 | -1.63 | -1.55 | 0.25 | -3.24 | 0.52 | -2.01 | -2.39 |
| | | YKSKFADLS | 57 | -0.74 | -0.07 | -1.00 | -1.03 | -1.16 | 0.72 | 1.31 | 0.07 | 0.38 | -0.38 | -0.61 | 1.49 | 0.17 | 0.12 | -0.13 | -0.40 |
| SKFADLSEAA | 40 | SKFADLSEA | 58 | 2.23 | 0.10 | -0.97 | -1.31 | -1.55 | 0.12 | -0.21 | -0.21 | 2.94 | -0.12 | -0.58 | 0.56 | -0.54 | 0.12 | -0.16 | 0.53 |
| | | KFADLSEAA | 59 | 1.46 | -1.29 | -2.19 | -2.34 | -1.70 | -2.60 | -2.74 | -2.32 | 0.31 | -2.33 | -2.76 | 0.12 | -0.37 | 0.29 | -0.41 | -0.58 |
| | | | | A | A | A | A | A | A | A | A | A | A | A | A | A | A | A | |
| | | 9-mer | | 2403 | 2501 | 2601 | 2602 | 2603 | 2902 | 3001 | 3002 | 3101 | 3201 | 3301 | 6801 | 6802 | 6901 | 8001 | |
| SLYASSPGGV | 29 | SLYASSPGG | 41 | 0.19 | 0.30 | 0.41 | 0.84 | -0.90 | 0.32 | -1.30 | -0.06 | 0.66 | 0.51 | 0.49 | 1.58 | -0.52 | -0.71 | 0.17 | |
| | | LYASSPGGV | 42 | -2.22 | 0.48 | 0.06 | -0.52 | -0.85 | -0.52 | 0.98 | 0.96 | 0.09 | -0.40 | 0.63 | 1.93 | -0.87 | -0.65 | 0.69 | |
| YASSPGGVY | 30 | YASSPGGVY | 43 | -0.73 | -1.33 | -2.50 | -1.65 | -1.41 | -2.16 | 0.81 | -1.92 | 1.06 | -0.51 | 0.41 | -1.15 | -0.33 | -0.50 | -3.52 | |
| SPGGVYATR | 31 | SPGGVYATR | 44 | -0.15 | -1.23 | -1.63 | -1.68 | -1.38 | -1.81 | 2.02 | -1.25 | -2.06 | 1.52 | -1.81 | -1.66 | 0.36 | 0.47 | -2.41 | |
| GVYATRSSAV | 32 | GVYATRSSA | 45 | -0.04 | 0.21 | -0.03 | -0.19 | -0.50 | 0.31 | -0.97 | 0.11 | 0.27 | 0.00 | 0.71 | 0.87 | -1.25 | -0.59 | -0.33 | |
| | | VYATRSSAV | 46 | -2.12 | 0.15 | 0.80 | -0.95 | -0.92 | -0.41 | 1.48 | 0.80 | 0.86 | -0.92 | 0.75 | 1.91 | -1.06 | -0.20 | 0.03 | |
| ELQELNDRFA | 33 | ELQELNDRF | 47 | -1.75 | -0.42 | -2.41 | -1.08 | 0.73 | -0.81 | 0.78 | 0.60 | 0.47 | -1.40 | 0.45 | -0.17 | -0.50 | -1.12 | -0.19 | |
| | | LQELNDRFA | 48 | 0.04 | 4.74 | 1.50 | 0.74 | 0.96 | -0.35 | -0.56 | 0.52 | 0.25 | -0.11 | 0.27 | 0.94 | -0.32 | -0.20 | -0.49 | |
| ELNDRFANY | 34 | ELNDRFANY | 49 | 0.17 | -4.57 | -4.51 | -2.67 | -1.15 | -2.14 | 1.19 | -1.07 | 0.39 | -0.62 | -0.86 | -0.66 | 0.49 | 0.58 | -2.15 | |
| RFANYIDKVR | 35 | RFANYIDKV | 50 | -3.18 | -1.80 | -0.46 | -0.39 | -0.26 | -0.51 | 0.51 | 1.75 | -0.63 | -1.41 | 0.13 | 1.37 | -1.35 | -2.53 | -0.15 | |
| | | FANYIDKVR | 51 | -0.08 | 0.09 | -0.24 | -0.59 | -0.60 | -2.17 | 0.10 | -1.32 | -2.24 | 0.63 | -2.18 | -2.59 | 0.22 | 1.10 | -2.20 | |
| NYIDKVRFL | 36 | NYIDKVRFL | 52 | -4.00 | 0.01 | -0.64 | -0.18 | 2.00 | -1.15 | 0.27 | 1.70 | -0.10 | -1.57 | 0.17 | 0.77 | 0.00 | -1.28 | -0.67 | |
| NLQEAEEWYK | 37 | NLQEAEEWY | 53 | -0.20 | 0.38 | -1.63 | -0.88 | 0.93 | -1.93 | -0.03 | 0.26 | 0.11 | -0.82 | 0.09 | -1.23 | 0.93 | -0.11 | -2.63 | |
| | | LQEAEEWYK | 54 | -0.27 | 2.30 | 1.17 | 1.00 | 2.89 | -1.39 | -0.88 | -1.33 | -1.83 | 0.24 | -0.36 | -0.99 | 1.64 | 0.73 | -0.52 | |
| EAEEWYKSK | 38 | EAEEWYKSK | 55 | 0.40 | -2.69 | -0.98 | -1.80 | -1.68 | -0.42 | -1.09 | -2.88 | -1.48 | 0.06 | -1.69 | -1.77 | 0.07 | -0.50 | -0.67 | |
| WYKSKFADLS | 39 | WYKSKFADL | 56 | -4.10 | -1.07 | 0.56 | 0.02 | 0.27 | -0.50 | 0.33 | 1.09 | 0.59 | -0.89 | 1.33 | 2.01 | 0.47 | 0.18 | 0.06 | |
| | | YKSKFADLS | 57 | -1.09 | 1.78 | 0.36 | 1.95 | 1.07 | 0.20 | -0.02 | 0.16 | 1.13 | 0.15 | 0.17 | 1.11 | 0.24 | -0.42 | -0.40 | |
| SKFADLSEAA | 40 | SKFADLSEA | 58 | -0.04 | 0.14 | 0.36 | 0.71 | -0.27 | 1.38 | 0.11 | 0.47 | 0.50 | 0.18 | 1.09 | 1.41 | 0.00 | -0.40 | 1.03 | |
| | | KFADLSEAA | 59 | -1.30 | 1.15 | 1.16 | 2.22 | 1.01 | 0.22 | -0.52 | 0.67 | -0.34 | -1.09 | 0.76 | 1.26 | -0.82 | -0.74 | -0.31 | |

NOTE
Binding is shown in standard deviation units comparing all peptides within the protein. While actual affinity varies between proteins a value of -1 SD equates to 100-200 nanomolar

TABLE 4
Selected vimentin MHC II epitope peptides and their binding

| | SEQ | | SEQ | | | | | | | | | | | | | | | | | |
|---|---|---|---|---|---|---|---|---|---|---|---|---|---|---|---|---|---|---|---|---|
| | ID | | ID | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 |
| peptide | NO.: | peptide | NO.: | 0101 | 0301 | 0401 | 0404 | 0405 | 0701 | 0801 | 0802 | 0901 | 1001 | 1101 | 1201 | 1301 | 1302 | 1454 | 1501 | 1602 |
| PSTSRSLYASSP | 60 | PSTSRSLYAS | 73 | −1.26 | 2.88 | −0.40 | −0.95 | 0.21 | 0.07 | 0.10 | −1.31 | −1.91 | −1.80 | −0.09 | 0.79 | 0.90 | 0.13 | 0.06 | −0.48 | −0.87 |
| GGVYA | | SPGGV | | | | | | | | | | | | | | | | | | |
| | | STSRSLYASS | 74 | −1.51 | 2.47 | −0.76 | −0.39 | 0.62 | −1.09 | 0.23 | −0.19 | −1.39 | −0.21 | 0.23 | −0.56 | 1.18 | 0.03 | −0.96 | 0.55 | −1.37 |
| | | PGGVY | | | | | | | | | | | | | | | | | | |
| | | TSRSLYASSP | 75 | −1.73 | 1.30 | −1.15 | −1.62 | −0.95 | −1.29 | 0.04 | −0.86 | −1.94 | −1.57 | 0.06 | 0.51 | 1.86 | −0.97 | −0.97 | −1.16 | −1.07 |
| | | GGVYA | | | | | | | | | | | | | | | | | | |
| SRSLYASSPGG | 61 | SRSLYASSPG | 76 | −2.83 | 1.04 | −1.01 | −1.23 | 0.37 | −1.17 | 0.24 | −0.85 | −2.04 | −1.90 | 0.45 | 0.42 | 1.53 | −1.47 | −0.77 | −0.69 | −1.74 |
| VYATRS | | GVYAT | | | | | | | | | | | | | | | | | | |
| | | RSLYASSPGG | 77 | −1.82 | 0.72 | −1.04 | −0.89 | −0.40 | −0.95 | 0.19 | −1.28 | −1.45 | −0.16 | 0.33 | −0.58 | −0.36 | −1.50 | −0.43 | −0.70 | −0.57 |
| | | VYATR | | | | | | | | | | | | | | | | | | |
| | | SLYASSPGGV | 78 | −1.67 | 1.15 | 1.05 | −0.93 | 0.02 | −0.64 | 0.93 | −0.21 | −0.78 | −2.72 | 0.96 | −0.35 | 0.34 | 0.90 | 0.15 | −0.25 | −1.56 |
| | | YATRS | | | | | | | | | | | | | | | | | | |
| SSAVRLRSSVP | 62 | SSAVRLRSSV | 79 | −0.86 | −0.30 | −0.76 | −0.99 | −0.69 | −1.24 | 0.35 | −1.02 | −1.20 | −0.08 | −0.15 | −0.94 | 0.53 | −0.56 | −0.63 | −1.29 | −0.79 |
| GVRLL | | PGVRL | | | | | | | | | | | | | | | | | | |
| | | SAVRLRSSVP | 80 | −2.13 | −0.03 | −0.60 | −1.50 | −0.94 | −1.86 | −1.00 | −0.05 | −1.80 | −1.23 | −0.45 | −0.97 | −1.15 | −1.51 | −1.51 | −0.96 | −1.15 |
| | | GVRLL | | | | | | | | | | | | | | | | | | |
| AVRLRSSVPGV | 63 | AVRLRSSVPG | 81 | −1.64 | −0.26 | −1.43 | −0.82 | −0.48 | −1.68 | −0.19 | −0.84 | −1.25 | −0.68 | −0.31 | −1.27 | 0.20 | −0.75 | −0.66 | −1.48 | −0.54 |
| RLLQD | | VRLLQ | | | | | | | | | | | | | | | | | | |
| | | VRLRSSVPGV | 82 | −1.51 | −0.18 | −0.75 | −0.29 | −0.10 | −0.95 | −0.26 | 0.43 | −1.55 | −1.64 | 0.45 | −0.97 | −0.96 | −0.67 | −0.51 | −0.38 | −0.66 |
| | | RLLQD | | | | | | | | | | | | | | | | | | |
| LNDRFANYIDKV | 64 | LNDRFANYID | 83 | −0.73 | −0.58 | −1.54 | −0.52 | −1.21 | −2.18 | −2.23 | 1.00 | −1.60 | −0.32 | −1.20 | −0.70 | −2.83 | −0.33 | −2.15 | −1.37 | −1.26 |
| RFLE | | KVRFL | | | | | | | | | | | | | | | | | | |
| | | NDRFANYIDK | 84 | −0.87 | −1.01 | 0.44 | −0.46 | 0.01 | −1.37 | −2.31 | −0.34 | −0.44 | 0.59 | −1.78 | −1.01 | −1.60 | −1.46 | −1.43 | −1.59 | −1.68 |
| | | VRFLE | | | | | | | | | | | | | | | | | | |
| DRFANYIDKVRF | 65 | DRFANYIDKV | 85 | −0.52 | −2.37 | −0.25 | −1.09 | −1.67 | −1.15 | −0.69 | −0.60 | −0.52 | −1.20 | −1.10 | −2.02 | −1.24 | −1.10 | −1.18 | −1.52 | 0.71 |
| LEQQ | | RFLEQ | | | | | | | | | | | | | | | | | | |
| | | RFANYIDKVR | 86 | −0.67 | −1.98 | −0.58 | −0.99 | −1.00 | −1.19 | −1.40 | −0.19 | −0.99 | 0.18 | −1.72 | 0.19 | −1.44 | −1.26 | −0.59 | −1.74 | −1.18 |
| | | FLEQQ | | | | | | | | | | | | | | | | | | |
| FANYIDKVRFLE | 66 | FANYIDKVRFL | 87 | −0.40 | −0.84 | −1.01 | −0.07 | −1.55 | −0.36 | −1.99 | −0.94 | −0.30 | 0.58 | −2.10 | −0.83 | −2.32 | 0.39 | −1.68 | −1.53 | 0.10 |
| QQNK | | EQQN | | | | | | | | | | | | | | | | | | |
| | | ANYIDKVRFLE | 88 | 0.22 | −0.20 | 0.00 | −0.22 | −0.98 | 0.51 | −0.69 | −0.04 | 0.01 | 0.47 | −1.52 | −1.66 | −1.22 | −0.44 | 0.28 | −0.84 | 3.11 |
| | | QQNK | | | | | | | | | | | | | | | | | | |
| NYIDKVRFLEQQ | 67 | NYIDKVRFLE | 89 | −0.74 | 0.07 | −0.42 | −1.50 | −1.18 | −1.45 | −1.93 | −0.70 | 0.12 | −0.82 | −2.32 | −1.00 | −2.04 | 0.90 | −0.73 | −0.76 | −0.47 |
| NKILL | | QQNKI | | | | | | | | | | | | | | | | | | |
| | | YIDKVRFLEQ | 90 | −0.34 | 0.00 | −2.09 | −1.37 | −1.24 | −1.26 | −2.50 | −0.38 | −0.61 | 0.61 | −1.79 | −1.50 | −1.88 | 0.23 | −1.40 | −0.57 | 1.13 |
| | | QNKIL | | | | | | | | | | | | | | | | | | |
| | | IDKVRFLEQQ | 91 | −1.11 | −1.29 | −0.90 | −1.04 | −0.97 | −1.81 | −1.94 | 0.42 | −0.27 | 0.12 | −1.20 | −1.39 | −1.28 | −0.98 | −2.42 | −1.36 | 0.91 |
| | | NKILL | | | | | | | | | | | | | | | | | | |
| DKVRFLEQQNKI | 68 | DKVRFLEQQN | 92 | −1.84 | −3.03 | −1.96 | −0.44 | −1.72 | −1.78 | −0.83 | −0.49 | −1.42 | −0.12 | −1.56 | 0.43 | −1.13 | −2.87 | −1.63 | −2.23 | −2.18 |
| LLAEL | | KILLA | | | | | | | | | | | | | | | | | | |
| | | KVRFLEQQNK | 93 | −2.01 | −1.37 | −1.50 | −0.70 | −1.41 | −2.10 | −1.33 | −2.38 | −1.66 | −0.98 | −1.55 | −0.93 | −0.59 | −1.72 | −0.99 | −2.45 | −2.04 |
| | | ILLAE | | | | | | | | | | | | | | | | | | |
| | | VRFLEQQNKIL | 94 | −1.87 | −2.38 | −1.22 | −1.25 | −1.44 | −2.16 | −1.27 | −1.42 | −1.53 | −0.32 | −1.34 | −1.39 | −1.66 | −2.27 | −1.46 | −0.99 | 0.26 |
| | | LAEL | | | | | | | | | | | | | | | | | | |
| FLEQQNKILLAE | 69 | FLEQQNKILLA | 95 | −0.88 | 0.96 | −0.72 | −0.85 | −1.62 | −1.21 | −0.77 | −0.84 | −1.09 | 0.19 | −0.62 | −1.06 | −0.30 | 0.34 | 0.60 | −1.42 | 1.29 |
| LEQL | | ELEQ | | | | | | | | | | | | | | | | | | |
| | | LEQQNKILLAE | 96 | −0.81 | 1.10 | −1.22 | −1.53 | −2.47 | −1.70 | −0.89 | 1.20 | −2.13 | 0.04 | −0.55 | −0.43 | −1.06 | −0.25 | −0.57 | −0.36 | 1.28 |
| | | LEQL | | | | | | | | | | | | | | | | | | |
| QQNKILLAELEQ | 70 | QQNKILLAELE | 97 | −1.62 | 0.19 | −1.74 | −1.28 | −2.35 | −1.33 | −1.28 | −0.20 | −1.20 | 0.22 | −0.94 | −0.03 | −0.56 | −0.69 | −0.57 | −2.63 | 0.05 |
| LKGQ | | QLKG | | | | | | | | | | | | | | | | | | |
| | | QNKILLAELEQ | 98 | −1.80 | −1.22 | −2.41 | −1.55 | −1.38 | −0.91 | −1.17 | 0.09 | −0.49 | −1.64 | −1.72 | −0.59 | −1.42 | −1.35 | −1.56 | −2.03 | −0.08 |
| | | LKGQ | | | | | | | | | | | | | | | | | | |
| NKILLAELEQLK | 71 | NKILLAELEQL | 99 | −1.81 | −0.86 | −1.27 | −1.24 | −2.26 | −0.69 | −2.08 | −1.61 | −0.46 | −0.53 | −1.26 | −0.72 | −1.34 | −1.47 | −1.53 | −1.49 | −2.82 |
| GQGK | | KGQG | | | | | | | | | | | | | | | | | | |
| | | KILLAELEQLK | 100 | −1.07 | 0.11 | −2.55 | −1.51 | −0.94 | −0.12 | −0.93 | −1.89 | −0.57 | −1.39 | −1.56 | −1.42 | −0.68 | −0.70 | −0.64 | −1.33 | −1.06 |
| | | GQGK | | | | | | | | | | | | | | | | | | |
| EAEEWYKSKFA | 72 | EAEEWYKSKF | 101 | −0.62 | 0.34 | −0.12 | 0.20 | −0.95 | −0.04 | −0.09 | −2.49 | −0.17 | −0.02 | −0.66 | −0.51 | 0.66 | 0.23 | 0.29 | −1.41 | 0.03 |
| DLSEAA | | ADLSE | | | | | | | | | | | | | | | | | | |
| | | AEEWYKSKFA | 102 | −1.36 | 0.95 | −1.14 | −1.01 | −1.64 | −0.28 | −0.42 | 0.47 | −1.36 | 0.78 | −1.79 | −0.02 | −0.60 | −1.50 | −0.64 | −0.35 | 0.31 |
| | | DLSEA | | | | | | | | | | | | | | | | | | |
| | | EEWYKSKFAD | 103 | −1.05 | 0.31 | −1.22 | −0.67 | −1.35 | −1.11 | −1.36 | −1.64 | −1.01 | −1.00 | −1.60 | 1.29 | −1.14 | 0.02 | −1.08 | −0.07 | −3.44 |
| | | LSEAA | | | | | | | | | | | | | | | | | | |

### Example 3: Epitope Peptides in WT-1

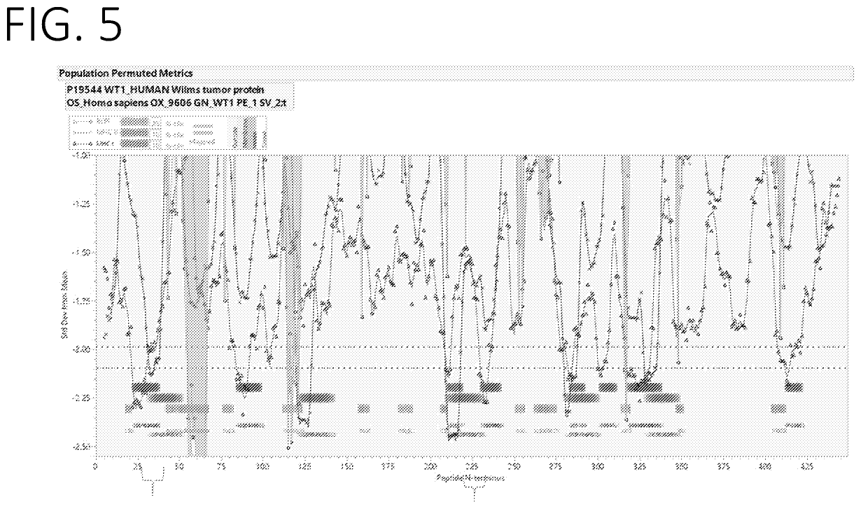

Wilms tumor protein WT1 is a transcription factor that plays an important role in cellular development and cell survival and is considered an oncogene. It is exemplified by sequence P19544. At least 8 additional isoforms are recognized and so epitopes are selected from the conserved regions. FIG. 5 provides an overview of the regions of highest MHC I and MHC II binding in WT1 with locations of selected sequences identified.

Tables 5 and 6 provide epitope peptides of WT1 which are predicted to bind multiple MHC I and MHC II alleles. A combination of 2 MHC I epitopes may be applied to provide broad coverage or a peptide may be selected based on the known HLA alleles of the subject. In preferred embodiments an MHC I epitope peptide is administered contemporaneously with an MHC II peptide that overlaps it and also binds to the alleles of the individual.

TABLE 5
Selected WT1 MHC I epitope peptides and their binding

| | SEQ | | SEQ | | | | | | | | | | | | | | | | |
|---|---|---|---|---|---|---|---|---|---|---|---|---|---|---|---|---|---|---|---|
| | ID | | ID | nA | nA | nA | nA | nA | nA | nA | nA | nA | nA | nA | nA | nA | nA | nA | nA |
| | NO.: | 9-mer | NO.: | 0101 | 0201 | 0202 | 0203 | 0206 | 0211 | 0212 | 0216 | 0217 | 0219 | 0250 | 0301 | 0801 | 1101 | 2301 | 2402 |
| QALLLRTPYS | 104 | QALLLRTPY | 106 | −0.36 | −0.01 | 0.26 | 1.13 | 0.59 | 0.40 | 0.73 | 1.14 | −2.36 | 0.62 | 2.07 | −1.44 | −0.56 | −2.10 | −0.05 | −0.05 |
| | | ALLLRTPYS | 107 | 0.42 | −2.33 | −0.74 | −1.42 | −0.91 | −1.12 | −1.60 | −1.61 | 0.61 | −1.46 | −0.91 | −0.12 | −0.50 | −0.50 | −0.94 | 0.17 |
| | | | | nA | nA | nA | nA | nA | nA | nA | nA | nA | nA | nA | nA | nA | nA | nA | |
| | | 9-mer | | 2403 | 2501 | 2601 | 2602 | 2603 | 2902 | 3001 | 3002 | 3101 | 3201 | 3301 | 6801 | 6802 | 6901 | 8001 | |
| QALLLRTPYS | 104 | QALLLRTPY | 106 | −0.46 | −1.52 | −2.40 | −1.98 | −1.50 | −2.74 | −0.05 | −3.31 | −0.99 | −1.55 | −1.09 | −1.60 | −0.18 | −0.62 | −3.18 | |
| | | ALLLRTPYS | 107 | 0.07 | 0.56 | 0.62 | 0.88 | −0.04 | 0.02 | −0.68 | −0.59 | −0.26 | 0.38 | 0.68 | 0.70 | 0.86 | 0.28 | 0.43 | |
| | | | | nB | nB | nB | nB | nB | nB | nB | nB | nB | nB | nB | nB | nB | nB | nB | |
| | | 9-mer | | 0702 | 0801 | 0802 | 0803 | 1501 | 1502 | 1503 | 1509 | 1517 | 1542 | 1801 | 2703 | 2705 | 3501 | 3801 | |
| QALLLRTPYS | 104 | QALLLRTPY | 106 | −0.56 | −0.05 | −0.42 | 0.20 | −2.15 | −1.48 | −0.04 | 0.76 | −2.35 | 0.14 | −0.59 | −0.53 | −0.34 | −1.76 | 0.34 | |
| | | ALLLRTPYS | 107 | 1.33 | −1.45 | 0.48 | −0.59 | 0.75 | 0.26 | 0.65 | −0.09 | −0.01 | −0.47 | 0.96 | −0.26 | 0.84 | 0.99 | 0.76 | |
| | | | | nB | nB | nB | nB | nB | nB | nB | nB | nB | B | nB | nB | nB | nB | nB | nB |
| | | 9-mer | | 3901 | 4001 | 4002 | 4402 | 4403 | 4501 | 4506 | 4601 | 4801 | 5101 | 5301 | 5401 | 5701 | 5801 | 7301 | 8301 |
| QALLLRTPYS | 104 | QALLLRTPY | 106 | 1.08 | 0.45 | 0.36 | 0.61 | −0.65 | 1.00 | 0.35 | −0.64 | −0.27 | −0.43 | −0.96 | −0.01 | −3.20 | −2.77 | 1.63 | −0.47 |
| | | ALLLRTPYS | 107 | 1.22 | 0.74 | 0.51 | 0.39 | −0.35 | −0.07 | 0.37 | 1.68 | 0.07 | 0.75 | 1.00 | −0.19 | 0.46 | 0.25 | −0.95 | −0.21 |
| | | | | nA | nA | nA | nA | nA | nA | nA | nA | nA | nA | nA | nA | nA | nA | nA | nA |
| | | 9-mer | | 0101 | 0201 | 0202 | 0203 | 0206 | 0211 | 0212 | 0216 | 0217 | 0219 | 0250 | 0301 | 0801 | 1101 | 2301 | 2402 |
| MFPNAPYLPS | 105 | MFPNAPYLP | 108 | −0.06 | −1.42 | −1.67 | −1.30 | −1.10 | −1.14 | −0.63 | −0.97 | −0.01 | −1.12 | −1.12 | −1.90 | −0.45 | −1.14 | −1.48 | −1.60 |
| | | FPNAPYLPS | 109 | −1.55 | −0.63 | −1.31 | −1.52 | −1.21 | −0.43 | 0.54 | −0.04 | 0.02 | −0.42 | −0.38 | 0.14 | −1.32 | −0.32 | −0.94 | −0.38 |
| | | | | nA | nA | nA | nA | nA | nA | nA | nA | nA | nA | nA | nA | nA | nA | nA | |
| | | 9-mer | | 2403 | 2501 | 2601 | 2602 | 2603 | 2902 | 3001 | 3002 | 3101 | 3201 | 3301 | 6801 | 6802 | 6901 | 8001 | |
| MFPNAPYLPS | 105 | MFPNAPYLP | 108 | −1.07 | −0.05 | −0.98 | 0.23 | 1.66 | −1.55 | −0.87 | −0.19 | −0.70 | 0.14 | −0.70 | −0.67 | −0.01 | −0.57 | −1.47 | |
| | | FPNAPYLPS | 109 | 0.73 | 0.67 | −0.10 | 0.07 | −0.35 | −0.38 | −1.04 | −0.19 | 0.23 | 0.61 | 0.26 | 1.32 | −0.50 | −0.64 | −0.47 | |
| | | | | nB | nB | nB | nB | nB | nB | nB | nB | nB | B | nB | nB | nB | nB | nB | |
| | | 9-mer | | 0702 | 0801 | 0802 | 0803 | 1501 | 1502 | 1503 | 1509 | 1517 | 1542 | 1801 | 2703 | 2705 | 3501 | 3801 | |
| MFPNAPYLPS | 105 | MFPNAPYLP | 108 | 0.25 | −0.30 | −0.32 | 0.27 | 0.11 | −0.77 | −0.75 | −0.40 | 0.67 | −0.58 | −0.53 | −0.16 | 0.12 | −0.19 | −0.05 | |
| | | FPNAPYLPS | 109 | −1.45 | −1.11 | 0.05 | −2.40 | 0.42 | −1.11 | 1.25 | 0.32 | 0.82 | −0.36 | 0.21 | −0.13 | −0.02 | −1.31 | −0.32 | |
| | | | | nB | nB | nB | nB | nB | nB | nB | nB | nB | nB | nB | nB | nB | nB | nB | nB |
| | | 9-mer | | 3901 | 4001 | 4002 | 4402 | 4403 | 4501 | 4506 | 4601 | 4801 | 5101 | 5301 | 5401 | 5701 | 5801 | 7301 | 8301 |
| MFPNAPYLPS | 105 | MFPNAPYLP | 108 | 0.44 | −0.24 | −0.39 | −0.55 | −1.02 | −0.51 | 0.26 | 0.20 | −0.19 | −0.59 | −0.35 | 0.18 | 0.79 | 0.00 | −1.14 | 0.89 |
| | | FPNAPYLPS | 109 | 0.20 | −0.04 | 0.42 | 0.03 | 0.71 | 0.12 | −0.14 | 0.63 | −0.04 | −1.58 | −0.85 | −2.91 | 1.68 | 0.77 | −0.76 | −0.34 |

NOTE:
Binding is shown in standard deviation units comparing all peptides within the protein. While actual affinity varies between proteins a value of −1 SD equates to 100-200 nanomolar

TABLE 6
Selected WT1 MHC II epitope peptides and their binding

| | SEQ | | SEQ | | | | | | | | | | | | | | | | |
|---|---|---|---|---|---|---|---|---|---|---|---|---|---|---|---|---|---|---|---|
| | ID | | ID | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 |
| | NO.: | | NO.: | 0101 | 0301 | 0401 | 0404 | 0405 | 0701 | 0801 | 0802 | 0901 | 1101 | 1201 | 1301 | 1302 | 1454 | 1501 | 1602 |
| SQALLLRTPYSS | 110 | SQALLLRTPYSSDNL | 113 | −1.19 | −1.58 | −1.83 | −1.72 | −1.21 | −1.01 | −2.22 | −2.03 | −0.75 | −2.11 | −1.38 | −1.93 | −0.74 | −1.74 | −2.18 | −0.51 |
| DNLYQ | | QALLLRTPYSSDNLY | 114 | −2.28 | −0.87 | −2.86 | −2.06 | −3.45 | −1.01 | −3.35 | −1.56 | −1.00 | −3.32 | −1.63 | −2.00 | −2.28 | −2.47 | −2.68 | 0.43 |
| | | ALLLRTPYSSDNLYQ | 115 | −1.42 | −0.19 | −0.84 | −1.62 | −1.29 | −1.47 | −2.05 | −1.42 | −1.81 | −2.52 | −1.23 | −1.97 | −1.27 | −2.01 | −1.30 | −1.05 |
| | | | | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 |
| | | | | 0101 | 0301 | 0401 | 0404 | 0405 | 0701 | 0801 | 0802 | 0901 | 1101 | 1201 | 1301 | 1302 | 1454 | 1501 | 1602 |
| QARMFPNAPYLP | 111 | QARMFPNAPYLPSCL | 116 | −1.59 | −0.90 | −1.42 | −1.88 | −2.12 | −1.46 | −1.37 | −1.14 | −1.74 | −1.15 | −1.55 | −2.17 | −2.98 | −1.62 | −2.34 | −0.23 |
| SCLES | | ARMFPNAPYLPSCLE | 117 | −1.78 | −0.88 | −1.47 | −1.26 | −2.01 | −2.76 | −1.35 | −1.17 | −2.23 | −1.76 | −1.86 | −0.83 | −0.34 | −1.25 | −1.36 | −1.88 |
| | | RMFPNAPYLPSCLES: | 118 | −1.36 | 1.05 | −0.57 | −1.06 | −1.66 | −1.80 | −1.98 | −1.31 | −1.03 | −1.60 | −1.07 | −1.30 | −0.09 | −2.83 | −1.08 | −0.73 |

NOTE:
Binding is shown in standard deviation units comparing all peptides within the protein. While actual affinity varies between proteins a value of −1 SD equates to 100-200 nanomolar

### Example 4: Common Mutations of EGFR in Glioblastoma and Lung Cancer

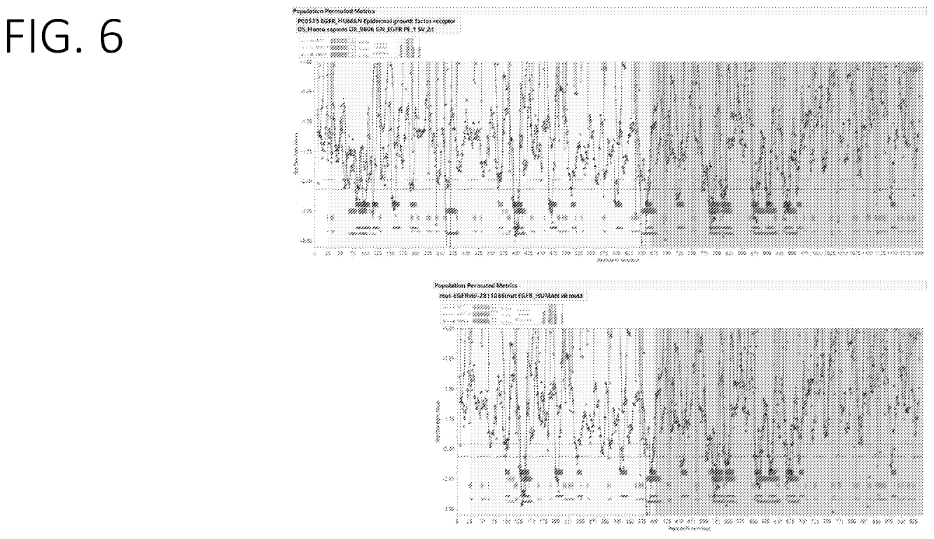

EGFR is a transmembrane protein with a transmembrane domain located at positions 646-667 relative to its N terminal and a signal peptide. The EGFRvIII variant found commonly in glioblastoma has a shortened extramembrane domain (FIG. 6) due to splice deletion of exons 2-7. Common mutations in glioblastoma include A289V/D/T, and G598D all located in the extramembrane domain. In lung cancer the most common mutation of EGFR is L858R. Each of these creates a novel amino acid motif which allows tumor specific targeting of T cells.

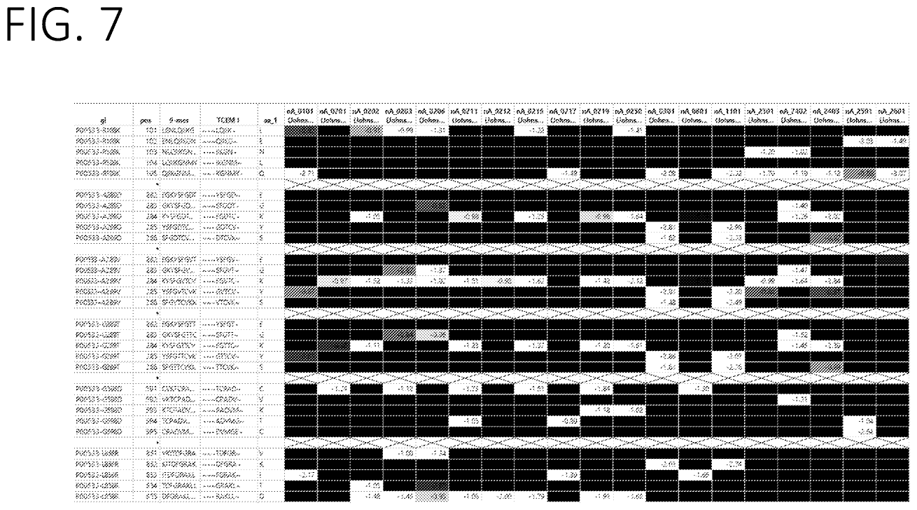

As shown in FIG. 7 for a subset of MHC IA alleles relatively few of the MHC I A alleles have moderate or high binding in positions which expose the mutant motifs, and some have excessively high binding which may result in anergy or exhaustion. This data is replicated in Table 7 below. Where natural binding falls in a stimulatory range, immunization with the neoepitope in its natural form may result in an effective cytotoxic response. In Table 8 we show examples of how modification of the flanking sequences can be used to generate a peptide with the same T cell exposed motif but also an appropriate binding affinity to generate a cytotoxic response. This is shown for a subset of MHC I A alleles, but such examples should not be considered limiting, as the same approach can be applied to other A alleles or to MHC IB or IC alleles.

Table 7 shows peptides which have T cell exposed motifs spanning the common EGFR mutations and in which natural binding affinity is appropriate; it will be noted that relatively few alleles have high affinity natural binding to the mutant T cell exposed motifs, reflecting one reason tumor motifs may achieve immune evasion.

Table 8 shows bespoke peptides as examples of peptides designed for exemplar alleles to provide binding that will allow them to be presented to these alleles where natural binding is insufficient to competitively stimulate a new T cell clone. As these examples were selected from a longer list for illustrative purposes, and other peptides with differing binding affinity could have been selected from that list, these examples are considered non limiting.

TABLE 7
Natural moderate-high affinity binding peptides in EGFR common mutants for exemplar MHC I A alleles

| | | | SEQ | | SEQ | | | | | | | | | | |
|---|---|---|---|---|---|---|---|---|---|---|---|---|---|---|---|
| | | | ID | | ID | A | A | A | A | A | A | A | A | A | A |
| gi | pos | 9-mer | NO.: | TCEM I | NO.: | 0101 | 0201 | 0202 | 0203 | 0206 | 0211 | 0212 | 0216 | 0217 | 0219 |
| P00533- | 282 | EGKYSFGDT | 124 | ~~~YSFGD~ | 154 | | | | | | | | | | |
| A289D | | | | | | | | | | | | | | | |
| P00533- | 283 | GKYSFGDTC | 125 | ~~~SFGDT~ | 155 | | | | | | | | | | |
| A289D | | | | | | | | | | | | | | | |
| P00533- | 284 | KYSFGDTCV | 126 | ~~~FGDTC~ | 156 | | | | | | | | −1.25 | | |
| A289D | | | | | | | | | | | | | | | |
| P00533- | 285 | YSFGDTCVK | 127 | ~~~GDTCV~ | 157 | | | | | | | | | | |
| A289D | | | | | | | | | | | | | | | |
| P00533- | 286 | SFGDTCVKK | 128 | ~~~DTCVK~ | 158 | | | | | | | | | | |
| A289D | | | | | | | | | | | | | | | |
| P00533- | 282 | EGKYSFGVT | 129 | ~~~YSFGV~ | 159 | | | | | | | | | | |
| A289V | | | | | | | | | | | | | | | |
| P00533- | 283 | GKYSFGVTC | 130 | ~~~SFGVT~ | 160 | | | | | −1.37 | | | | | |
| A289V | | | | | | | | | | | | | | | |
| P00533- | 284 | KYSFGVTCV | 131 | ~~~FGVTC~ | 161 | | | −1.52 | −1.33 | −1.02 | −1.51 | | −1.62 | | −1.42 |
| A289V | | | | | | | | | | | | | | | |
| P00533- | 285 | YSFGVTCVK | 132 | ~~~GVTCV~ | 162 | | | | | | | | | | |
| A289V | | | | | | | | | | | | | | | |
| P00533- | 286 | SFGVTCVKK | 133 | ~~~VTCVK~ | 163 | | | | | | | | | | |
| A289V | | | | | | | | | | | | | | | |
| P00533- | 282 | EGKYSFGTT | 134 | ~~~YSFGT~ | 164 | | | | | | | | | | |
| G289T | | | | | | | | | | | | | | | |
| P00533- | 283 | GKYSFGTTC | 135 | ~~~SFGTT~ | 165 | | | | | | | | | | |
| G289T | | | | | | | | | | | | | | | |
| P00533- | 284 | KYSFGTTCV | 136 | ~~~FGTTC~ | 166 | | | | | | −1.23 | | −1.37 | | −1.20 |
| G289T | | | | | | | | | | | | | | | |
| P00533- | 285 | YSFGTTCVK | 137 | ~~~GTTCV~ | 167 | | | | | | | | | | |
| G289T | | | | | | | | | | | | | | | |
| P00533- | 286 | SFGTTCVKK | 138 | ~~~TTCVK~ | 168 | | | | | | | | | | |
| G289T | | | | | | | | | | | | | | | |
| P00533- | 591 | CVKTCPADV | 139 | ~~~TCPAD~ | 169 | | −1.24 | | −1.12 | | −1.73 | | −1.51 | | −1.84 |
| G598D | | | | | | | | | | | | | | | |
| P00533- | 592 | VKTCPADVM | 140 | ~~~CPADV~ | 170 | | | | | | | | | | |
| G598D | | | | | | | | | | | | | | | |
| P00533- | 593 | KTCPADVMG | 141 | ~~~PADVM~ | 171 | | | | | | | | | | −1.18 |
| G598D | | | | | | | | | | | | | | | |
| P00533- | 594 | TCPADVMGE | 142 | ~~~ADVMG~ | 172 | | | | | | | | | | |
| G598D | | | | | | | | | | | | | | | |
| P00533- | 595 | CPADVMGEN | 143 | ~~~DVMGE~ | 173 | | | | | | | | | | |
| G598D | | | | | | | | | | | | | | | |
| P00533- | 591 | CVKTCPAVV | 144 | ~~~TCPAV~ | 174 | | −1.52 | | −1.19 | | −1.87 | | −1.57 | | −2.00 |
| G598V | | | | | | | | | | | | | | | |
| P00533- | 592 | VKTCPAVVM | 145 | ~~~CPAVV~ | 175 | | | | | | | | | | |
| G598V | | | | | | | | | | | | | | | |
| P00533- | 593 | KTCPAVVMG | 146 | ~~~PAVVM~ | 176 | | −1.27 | | | −1.07 | | | | | −1.50 |
| G598V | | | | | | | | | | | | | | | |
| P00533- | 594 | TCPAVVMGE | 147 | ~~~AVVMG~ | 177 | | | | | | −1.04 | | | | |
| G598V | | | | | | | | | | | | | | | |
| P00533- | 595 | CPAVVMGEN | 148 | ~~~VVMGE~ | 178 | | | | | | | | | | |
| G598V | | | | | | | | | | | | | | | |
| P00533- | 851 | VKITDFGRA | 149 | ~~~TDFGR~ | 179 | | | | | −1.34 | | | | | |
| L858R | | | | | | | | | | | | | | | |
| P00533- | 852 | KITDFGRAK | 150 | ~~~DFGRA~ | 180 | | | | | | | | | | |
| L858R | | | | | | | | | | | | | | | |
| P00533- | 853 | ITDFGRAKL | 151 | ~~~FGRAK~ | 181 | −2.17 | | | | | | | | −1.39 | |
| L858R | | | | | | | | | | | | | | | |
| P00533- | 854 | TDFGRAKLL | 152 | ~~~GRAKL~ | 182 | | | −1.05 | | | | | | | |
| L858R | | | | | | | | | | | | | | | |
| P00533- | 855 | DFGRAKLLG | 153 | ~~~RAKLL~ | 183 | | | −1.48 | −1.45 | | −1.06 | −2.00 | −1.79 | | −1.91 |
| L858R | | | | | | | | | | | | | | | |

NOTE:
Binding is shown in standard deviation units comparing all peptides within the protein. While actual affinity varies between proteins a value of −1 SD equates to 100-200 nanomolar

TABLE 8
Peptides designed to provide targeting of EGFR common mutants for exemplar MHC I A alleles by modification of the
pocket positions

| | | | | | | | | Binding affinity of | | | Binding affinity of | |
|---|---|---|---|---|---|---|---|---|---|---|---|---|
| | | | SEQ | | | | | proposed peptide | | | originating peptide | |
| | | originating | ID | proposed | SEQ ID | | SEQ ID | A_0202 | A_2601 | | A | A |
| gi | pos | peptide | NO.: | peptide | NO.: | TCEM I | NO.: | mean | mean | polarity | 0202 | 2601 |
| P00533- | 282 | EGKYSFGDT | 124 | KVTYSFGDY | 196 | ~~~YSFGD~ | 154 | | −2.02 | −0.05 | | −0.44 |
| A289D | | | | | | | | | | | | |
| P00533- | 282 | EGKYSFGDT | 124 | YTYYSFGDI | 197 | ~~~YSFGD~ | 154 | | −2.02 | 1.19 | | −0.44 |
| A289D | | | | | | | | | | | | |
| P00533- | 283 | GKYSFGDTC | 125 | YKYSFGDTI | 198 | ~~~SFGDT~ | 155 | −2.03 | | 0.16 | −0.52 | |
| A289D | | | | | | | | | | | | |
| P00533- | 283 | GKYSFGDTC | 125 | EYCSFGDTI | 199 | ~~~SFGDT~ | 155 | −2.03 | | 0.22 | −0.52 | |
| A289D | | | | | | | | | | | | |
| P00533- | 284 | KYSFGDTCV | 126 | YVFGDTCI | 200 | ~~~FGDTC~ | 156 | −1.88 | | 1.96 | −1.05 | |
| A289D | | | | | | | | | | | | |
| P00533- | 284 | KYSFGDTCV | 126 | YYTFGDTCV | 201 | ~~~FGDTC~ | 156 | −1.84 | | 1.07 | −1.05 | |
| A289D | | | | | | | | | | | | |
| P00533- | 285 | YSFGDTCVK | 127 | YSVGDTCVY | 202 | ~~~GDTCV~ | 157 | | −2.02 | 0.66 | | −0.37 |
| A289D | | | | | | | | | | | | |
| P00533- | 285 | YSFGDTCVK | 127 | KVVGDTCVY | 203 | ~~~GDTCV~ | 157 | | −2.01 | 0.46 | | −0.37 |
| A289D | | | | | | | | | | | | |
| P00533- | 286 | SFGDTCVKK | 128 | YVCDTCVKV | 204 | ~~~DTCVK~ | 158 | −2.02 | | 0.78 | −0.31 | |
| A289D | | | | | | | | | | | | |
| P00533- | 286 | SFGDTCVKK | 128 | IYTDTCVKS | 205 | ~~~DTCVK~ | 158 | −2.01 | | −0.23 | −0.31 | |
| A289D | | | | | | | | | | | | |
| P00533- | 282 | EGKYSFGTT | 134 | EGVYSFGTE | 206 | ~~~YSFGT~ | 164 | | −2.02 | −0.27 | | −0.61 |
| G289T | | | | | | | | | | | | |
| P00533- | 282 | EGKYSFGTT | 134 | DVVYSFGTT | 207 | ~~~YSFGT~ | 164 | | −2.01 | 0.78 | | −0.61 |
| G289T | | | | | | | | | | | | |
| P00533- | 283 | GKYSFGTTC | 135 | EYDSFGTTV | 208 | ~~~SFGTT~ | 165 | −2.00 | | −0.38 | −0.51 | |
| G289T | | | | | | | | | | | | |
| P00533- | 283 | GKYSFGTTC | 135 | IIGSFGTTS | 209 | ~~~SFGTT~ | 165 | −2.00 | | 1.20 | −0.51 | |
| G289T | | | | | | | | | | | | |
| P00533- | 284 | KYSFGTTCV | 136 | YYVFGTTCV | 210 | ~~~FGTTC~ | 166 | −2.08 | | 2.27 | −1.11 | |
| G289T | | | | | | | | | | | | |
| P00533- | 284 | KYSFGTTCV | 136 | YYIFGTTCI | 211 | ~~~FGTTC~ | 166 | −2.00 | | 2.68 | −1.11 | |
| G289T | | | | | | | | | | | | |
| P00533- | 285 | YSFGTTCVK | 137 | EVVGTTCVV | 212 | ~~~GTTCV~ | 167 | | −2.05 | 1.33 | | −0.37 |
| G289T | | | | | | | | | | | | |
| P00533- | 285 | YSFGTTCVK | 137 | VTGGTTCVY | 213 | ~~~GTTCV~ | 167 | | −2.01 | 0.94 | | −0.37 |
| G289T | | | | | | | | | | | | |
| P00533- | 591 | CVKTCPADV | 139 | ETCTCPADT | 214 | ~~~TCPAD~ | 169 | | −2.01 | −1.23 | | −0.71 |
| G598D | | | | | | | | | | | | |
| P00533- | 591 | CVKTCPADV | 139 | TTETCPADY | 215 | ~~~TCPAD~ | 169 | | −2.01 | −1.18 | | −0.71 |
| G598D | | | | | | | | | | | | |
| P00533- | 591 | CVKTCPADV | 139 | KYKTCPADV | 216 | ~~~TCPAD~ | 169 | −2.01 | | −1.18 | −0.45 | |
| G598D | | | | | | | | | | | | |
| P00533- | 591 | CVKTCPADV | 139 | GYDTCPADV | 217 | ~~~TCPAD~ | 169 | −2.01 | | −0.46 | −0.45 | |
| G598D | | | | | | | | | | | | |
| P00533- | 595 | CPADVMGEN | 143 | EGVDVMGEK | 218 | ~~~DVMGE~ | 173 | | −2.00 | −1.32 | | −0.64 |
| G598D | | | | | | | | | | | | |
| P00533- | 595 | CPADVMGEN | 143 | DKIDVMGEY | 219 | ~~~DVMGE~ | 173 | | −2.00 | −0.78 | | −0.64 |
| G598D | | | | | | | | | | | | |
| P00533- | 591 | CVKTCPAVV | 144 | TSGTCPAVY | 220 | ~~~TCPAV~ | 174 | | −2.03 | 0.43 | | −0.86 |
| G598V | | | | | | | | | | | | |
| P00533- | 591 | CVKTCPAVV | 144 | DSSTCPAVY | 221 | ~~~TCPAV~ | 174 | | −2.03 | −0.29 | | −0.86 |
| G598V | | | | | | | | | | | | |
| P00533- | 591 | CVKTCPAVV | 144 | KYITCPAVG | 222 | ~~~TCPAV~ | 174 | −2.01 | | 0.88 | −0.69 | |
| G598V | | | | | | | | | | | | |
| P00533- | 591 | CVKTCPAVV | 144 | EYSTCPAVV | 223 | ~~~TCPAV~ | 174 | −2.01 | | 0.58 | −0.69 | |
| G598V | | | | | | | | | | | | |
| P00533- | 592 | VKTCPAVVM | 145 | DCCCPAVVY | 224 | ~~~CPAVV~ | 175 | | −2.02 | 1.46 | | −0.34 |
| G598V | | | | | | | | | | | | |
| P00533- | 592 | VKTCPAVVM | 145 | VVKCPAVVY | 225 | ~~~CPAVV~ | 175 | | −2.01 | 1.98 | | −0.34 |
| G598V | | | | | | | | | | | | |
| P00533- | 592 | VKTCPAVVM | 145 | TIKCPAVVV | 226 | ~~~CPAVV~ | 175 | −2.06 | | 1.75 | −0.38 | |
| G598V | | | | | | | | | | | | |
| P00533- | 592 | VKTCPAVVM | 145 | GIECPAVVV | 227 | ~~~CPAVV~ | 175 | −2.03 | | 1.93 | −0.38 | |
| G598V | | | | | | | | | | | | |
| P00533- | 593 | KTCPAVVMG | 146 | KYKPAVVMI | 228 | ~~~PAVVM~ | 176 | −2.03 | | 1.14 | −0.44 | |
| G598V | | | | | | | | | | | | |
| P00533- | 593 | KTCPAVVMG | 146 | KYKPAVVMV | 229 | ~~~PAVVM~ | 176 | −2.02 | | 0.94 | −0.44 | |
| G598V | | | | | | | | | | | | |
| P00533- | 595 | CPAVVMGEN | 148 | EVGVVMGEK | 230 | ~~~VVMGE~ | 178 | | −2.03 | −0.12 | | −0.41 |
| G598V | | | | | | | | | | | | |
| P00533- | 595 | CPAVVMGEN | 148 | EDVVVMGEY | 231 | ~~~VVMGE~ | 178 | | −2.02 | 0.32 | | −0.41 |
| G598V | | | | | | | | | | | | |
| P00533- | 851 | VKITDFGRA | 149 | EVSTDFGRK | 232 | ~~~TDFGR~ | 179 | | −2.03 | −1.95 | | −0.34 |
| L858R | | | | | | | | | | | | |
| P00533- | 851 | VKITDFGRA | 149 | GVETDFGRY | 233 | ~~~TDFGR~ | 179 | | −2.00 | −0.71 | | −0.34 |
| L858R | | | | | | | | | | | | |
| P00533- | 851 | VKITDFGRA | 149 | TYGTDFGRG | 234 | ~~~TDFGR~ | 179 | −2.01 | | −0.86 | −0.68 | |
| L858R | | | | | | | | | | | | |
| P00533- | 851 | VKITDFGRA | 149 | IIKTDFGRI | 235 | ~~~TDFGR~ | 179 | −2.01 | | 0.66 | −0.68 | |
| L858R | | | | | | | | | | | | |
| P00533- | 853 | ITDFGRAKL | 151 | DTIFGRAKI | 236 | ~~~FGRAK~ | 181 | | −2.02 | 0 | | −0.05 |
| L858R | | | | | | | | | | | | |
| P00533- | 853 | ITDFGRAKL | 151 | EVSFGRAKI | 237 | ~~~FGRAK~ | 181 | | −2.01 | −0.31 | | −0.05 |
| L858R | | | | | | | | | | | | |
| P00533- | 853 | ITDFGRAKL | 151 | YYTFGRAKI | 238 | ~~~FGRAK~ | 181 | −1.98 | | 0.50 | −0.64 | |
| L858R | | | | | | | | | | | | |
| P00533- | 853 | ITDFGRAKL | 151 | YYVFGRAKI | 239 | ~~~FGRAK~ | 181 | −1.94 | | 1.19 | −0.64 | |
| L858R | | | | | | | | | | | | |
| P00533- | 854 | TDFGRAKLL | 152 | SIGGRAKLV | 240 | ~~~GRAKL~ | 182 | −2.01 | | 0.18 | −1.05 | |
| L858R | | | | | | | | | | | | |
| P00533- | 854 | TDFGRAKLL | 152 | EYYGRAKLI | 241 | ~~~GRAKL~ | 182 | −2.01 | | −0.04 | −1.05 | |
| L858R | | | | | | | | | | | | |
| P00533- | 855 | DFGRAKLLG | 153 | KYTRAKLLI | 242 | ~~~RAKLL~ | 183 | −2.01 | | 0.23 | −1.48 | |
| L858R | | | | | | | | | | | | |
| P00533- | 855 | DFGRAKLLG | 153 | GYGRAKLLV | 243 | ~~~RAKLL~ | 183 | −2.01 | | 0.75 | −1.48 | |
| L858R | | | | | | | | | | | | |

NOTE:
Binding is shown in standard deviation units comparing all peptides within the protein. While actual affinity varies between proteins a value of −1 SD equates to 100-200 nanomolar

### Example 5: Bespoke Peptides Spanning the Oncogenic Deletion in EGFRvIII

Unique T cell exposed motifs span the deletion junction in EGFRvIII, however relatively few MHC I alleles bind at least one of the five possible unique T cell exposed motifs. Overall 31 of 70 MHC I alleles bound at less than −500 nM (1 SD), comprising 17 binding sites among the 31 B alleles, 9 of 31 A allele and 5 of 8 C alleles evaluated had binding less than 500 nM at any of the possible T cell exposed motifs. In particular, no binding of A0201 was predicted. In addition, A0101, B4001 and B 1542 had predicted binding in excess of 2.75 SD below the mean equivalent of approximately 20 nM which may be an affinity so high it could induce suppression or exhaustion. Therefore, EGFRvIII is a candidate for designing peptides specifically to optimize binding to a patient's alleles. Among the 70 alleles for which predicted binding was evaluated in the natural mutated EGFRvIII, 65 alleles have some probability of presentation of the native epitope based on at least a low level of binding of the natural peptide. These are candidates for using a synthetic bespoke peptide to stimulate T cells which are cognate for and can therefore target these T cell exposed motifs. Following the process laid out in the prior examples we generated a set of 10,000 peptides for each of the possible T cell exposed positions EEKKG (SEQ ID NO: 284), EKKGN (SEQ ID NO: 285), KKGNY (SEQ ID NO: 286), KGNYV (SEQ ID NO: 287), GNYVV (SEQ ID NO: 288).

Examples of peptides with selected predicted binding affinity are shown in Table 9 for a set of example alleles. These are assigned SEQ ID NOs.: 244-283. As many different peptides can be designed with flanking regions that produce the desired binding affinity and solubility, these examples shown are provided as illustrative but non-limiting examples.

TABLE 9
Exemplar Bespoke peptides spanning the oncogenic deletion in EGFRvIII

| | | | | Predicted | | | |
|---|---|---|---|---|---|---|---|
| Binding | | | SEQ | binding | Polarity/ | TCEM core | SEQ |
| group | Allele | Peptide | ID NO: | in SD units | solubility | amino acids | ID NO.: |
| High | A0101 | LADKKGNYV | 244 | −2.59 | −1.09 | KKGNY | 286 |
| | A0101 | KASEKKGNY | 245 | −2.57 | −3.36 | EKKGN | 285 |
| | A0101 | DGDGNYVVS | 246 | −2.55 | −0.94 | GNYVV | 288 |
| | A0201 | KLAEKKGNV | 247 | −2.67 | −2.08 | EKKGN | 285 |
| | A2402 | QYTKKGNYF | 248 | −2.72 | −1.28 | KKGNY | 286 |
| | A2402 | KYTKGNYVW | 249 | −2.67 | −0.47 | KGNYV | 287 |
| | A6901 | ESDKGNYVC | 250 | −2.54 | −1.86 | KGNYV | 287 |
| | B0702 | APGEEKKGG | 251 | −2.66 | −2.93 | EEKKG | 284 |
| | B0702 | PPDKGNYVA | 252 | −2.64 | −1.09 | KGNYV | 287 |
| | B3501 | LLREEKKGF | 253 | −2.62 | −1.27 | EEKKG | 284 |
| | B3501 | FAMEKKGNY | 254 | −2.57 | −1.06 | EKKGN | 285 |
| | B4402 | ECRKGNYVE | 255 | −2.72 | −2.22 | KGNYV | 287 |
| | B4402 | PCQKKGNYV | 256 | −2.72 | −1.44 | KKGNY | 286 |
| | B5701 | LGDEKKGNF | 257 | −2.66 | −1.91 | EKKGN | 285 |
| | B5701 | PASEEKKGF | 258 | −2.65 | −2.25 | EEKKG | 284 |
| | C0401 | IRQKGNYVS | 259 | −2.65 | −1.19 | KGNYV | 287 |
| | C0401 | LWSEKKGNG | 260 | −2.64 | −1.70 | EKKGN | 285 |
| | C0602 | TKSKKGNYR | 261 | −2.74 | −3.66 | KKGNY | 286 |
| | C0602 | IRRGNYVVS | 262 | −2.66 | −0.17 | GNYVV | 288 |
| | C0602 | LKEEEKKGD | 263 | −2.23 | −4.15 | EEKKG | 284 |
| Medium | A0101 | RAEGNYVVR | 264 | −2.01 | −1.17 | GNYVV | 288 |
| | A0101 | MGEKKGNYD | 265 | −2.01 | −2.69 | KKGNY | 286 |
| | A0101 | TADEKKGNF | 266 | −2.01 | −2.64 | EKKGN | 285 |
| | A0201 | RLKEKKGNV | 267 | −1.99 | −2.86 | EKKGN | 285 |
| | A2402 | QLPKKGNYI | 268 | −2.00 | −0.74 | KKGNY | 286 |
| | A2402 | TKGKGNYVI | 269 | −2.00 | −0.74 | KGNYV | 287 |
| | A6901 | EVSKGNYVA | 270 | −2.00 | −0.81 | KGNYV | 287 |
| | B0702 | NVRKGNYVA | 271 | −1.99 | −1.06 | KGNYV | 287 |
| | B0702 | RTQEEKKGI | 272 | −1.99 | −3.40 | EEKKG | 284 |
| | B3501 | QSCEKKGNW | 273 | −2.00 | −2.55 | EKKGN | 285 |
| | B3501 | FPMEEKKGR | 274 | −1.99 | −2.28 | EEKKG | 284 |
| | B4402 | SEEKKGNYQ | 275 | −2.00 | −3.77 | KKGNY | 286 |
| | B4402 | LELKGNYVP | 276 | −2.00 | 0.34 | KGNYV | 287 |
| | B5701 | EGPEEKKGY | 277 | −2.00 | −3.27 | EEKKG | 284 |
| | B5701 | ISKEKKGNF | 278 | −1.99 | −2.18 | EKKGN | 285 |
| | C0401 | EHMKGNYVG | 279 | −2.01 | −1.03 | KGNYV | 287 |
| | C0401 | RELEKKGNA | 280 | −2.000 | −3.21 | EKKGN | 285 |
| | C0602 | AEHGNYVVT | 281 | −2.01 | −0.21 | GNYVV | 288 |
| | C0602 | TRVKKGNYS | 282 | −2.01 | −2.39 | KKGNY | 286 |
| | C0602 | WKEEEKKGR | 283 | −2.01 | −4.28 | EEKKG | 284 |

NOTE:
Binding is shown in standard deviation units comparing all peptides within the protein. While actual affinity varies between proteins a value of −1 SD equates to 100-200 nanomolar

### Example 6: R132H Mutants of Isocitrate Dehydrogenase IDH1

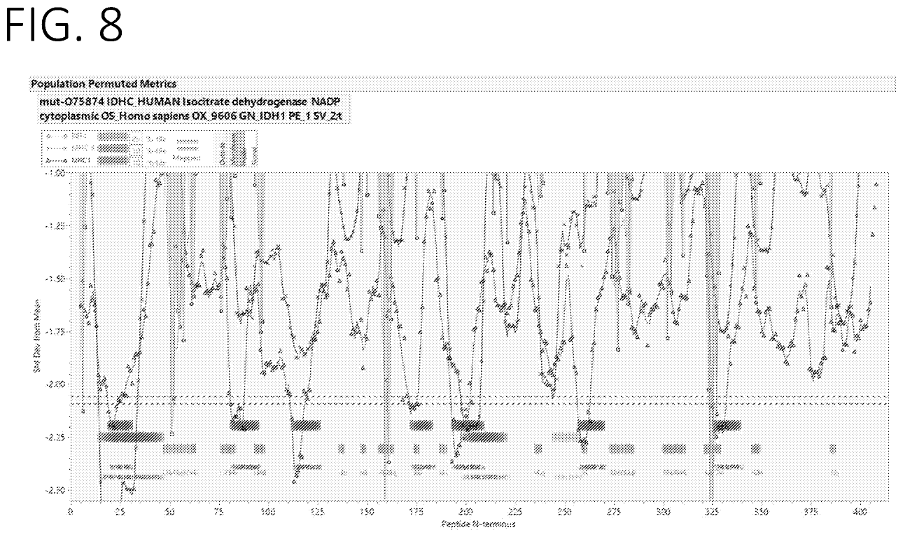

Isocitrate dehydrogenase IDH1, encoded by sequence 075874, is commonly mutated in gliomas. The most frequent mutation is R132H is associated with loss of conversion of isocitrate to alpha-ketoglutarate and increased risk of hypermethylation. FIG. 8 provides an overview of MHC binding regions in IDH1 R132H. The R132H mutation produces novel tumor specific class I T cell exposed motifs ˜˜˜IIIGH˜ (SEQ ID NO.: 294), ˜˜˜IIGHH˜ (SEQ ID NO.: 295), ˜˜˜IGHHA˜ (SEQ ID NO.: 296), ˜˜˜GHHAY˜ (SEQ ID NO.: 297), ˜˜˜HHAYG˜ (SEQ ID NO.: 298). For a few MHC I alleles, namely A0201, A0202, A0203, A0206, A0211, A0212, A0216 and A0250 the 9-mer peptide IIIGHHAYG (SEQ ID NO.: 292) encompassing ˜˜˜GHHAY˜ (SEQ ID NO.: 297) provides a predicted binding affinity of a suitable range to stimulate T cells, approximating to 100-200 nmolar. For these alleles the natural peptide may provide a suitable immunogen. In this case the natural 15mer VKPIIIGHHAYGDQY (SEQ ID NO.: 341) may be administered to provide CD4+ help, albeit at a more moderate binding affinity.

For other alleles and to increase the array of alleles the generation of designed flanking regions to the above cited T cell exposed motifs is desirable. Tables 10 and 11 provide examples of such bespoke peptides for an illustrative set of alleles. These examples are considered non limiting as the same approach can be applied for other alleles and multiple peptide options exist for each allele.

TABLE 10
Exemplars of peptides designed to provide high binding affinity for various MHC I alleles to
the tumor specific T cell exposed motifs of IDH R132H

| | origin- | SEQ | | SEQ | | SEQ | | Proposed | original | |
|---|---|---|---|---|---|---|---|---|---|---|
| | ating | ID | proposed | ID | TCEM | ID | | peptide | peptide | |
| pos | peptide | NO.: | peptide | NO.: | core | NO.: | Allele | affinity | affinity | polarity |
| 125 | VKPIIIGHH | 289 | HGDIIIGHK | 299 | IIIGH | 294 | A0101 | −2.02 | −0.27 | 0.07 |
| 125 | VKPIIIGHH | 289 | KADIIIGHK | 300 | IIIGH | 294 | A0101 | −2.01 | −0.27 | −0.23 |
| 125 | VKPIIIGHH | 289 | EWKIIIGHI | 301 | IIIGH | 294 | A2402 | 2.04 | −0.69 | 1.84 |
| 125 | VKPIIIGHH | 289 | KYTIIIGHL | 302 | IIIGH | 294 | A2402 | −2.02 | −0.69 | 1.97 |
| 125 | VKPIIIGHH | 289 | NVEIIIGHR | 303 | IIIGH | 294 | A6801 | −2.01 | −0.60 | 0.56 |
| 125 | VKPIIIGHH | 289 | YNAIIIGHK | 304 | IIIGH | 294 | A6801 | −2.01 | −0.60 | 0.99 |
| 125 | VKPIIIGHH | 289 | ARPIIIGHN | 305 | IIIGH | 294 | B2705 | −2.03 | −0.48 | 0.74 |
| 125 | VKPIIIGHH | 289 | KKMIIIGHA | 306 | IIIGH | 294 | B2705 | −2.02 | −0.48 | 0.90 |
| 126 | KPIIIGHHA | 290 | QSDIIGHHR | 307 | IIGHH | 295 | A0101 | −2.02 | −0.15 | −1.28 |
| 126 | KPIIIGHHA | 290 | NGDIIGHHS | 308 | IIGHH | 295 | A0101 | −2.01 | −0.15 | −0.71 |
| 126 | KPIIIGHHA | 290 | NGQIIGHHI | 309 | IIGHH | 295 | B5701 | −2.01 | −0.11 | 0.56 |
| 126 | KPIIIGHHA | 290 | RGNIIGHHR | 310 | IIGHH | 295 | B5701 | −2.00 | −0.11 | −1.14 |
| 127 | PIIIGHHAY | 291 | YSDIGHHAK | 311 | IGHHA | 296 | A0101 | −2.04 | −1.20 | −1.25 |
| 127 | PIIIGHHAY | 291 | KGDIGHHAL | 312 | IGHHA | 296 | A0101 | −2.03 | −1.20 | −0.60 |
| 127 | PIIIGHHAY | 291 | EYLIGHHAF | 313 | IGHHA | 296 | A2402 | −2.08 | −0.68 | 1.35 |
| 127 | PIIIGHHAY | 291 | DWRIGHHAF | 314 | IGHHA | 296 | A2402 | −2.01 | −0.68 | 0.23 |
| 127 | PIIIGHHAY | 291 | EVLIGHHAP | 315 | IGHHA | 296 | A2601 | −2.02 | −1.53 | 0.80 |
| 127 | PIIIGHHAY | 291 | QPFIGHHAY | 316 | IGHHA | 296 | A2601 | −2.02 | −1.53 | 0.81 |
| 127 | PIIIGHHAY | 291 | EINIGHHAK | 317 | IGHHA | 296 | A6801 | −2.01 | −0.70 | −0.95 |
| 127 | PIIIGHHAY | 291 | IADIGHHAK | 318 | IGHHA | 296 | A6801 | −2.01 | −0.70 | −0.43 |
| 127 | PIIIGHHAY | 291 | QRKIGHHAT | 319 | IGHHA | 296 | B2705 | −2.02 | −0.09 | −1.87 |
| 127 | PIIIGHHAY | 291 | SRIIGHHAS | 320 | IGHHA | 296 | B2705 | −2.02 | −0.09 | −0.38 |
| 127 | PIIIGHHAY | 291 | AELIGHHAN | 321 | IGHHA | 296 | B4402 | −2.01 | −0.29 | −0.14 |
| 127 | PIIIGHHAY | 291 | GEYIGHHAE | 322 | IGHHA | 296 | B4402 | −2.00 | −0.29 | −0.90 |
| 127 | PIIIGHHAY | 291 | QAKIGHHAY | 323 | IGHHA | 296 | B5701 | −2.05 | −1.10 | −0.65 |
| 127 | PIIIGHHAY | 291 | EQSIGHHAW | 324 | IGHHA | 296 | B5701 | −2.02 | −1.10 | −0.57 |
| 128 | IlIGHHAYG | 292 | LDDGHHAYA | 325 | GHHAY | 297 | A0101 | −2.03 | −0.25 | −0.82 |
| 128 | IIIGHHAYG | 292 | RADGHHAYA | 326 | GHHAY | 297 | A0101 | −2.01 | −0.25 | −1.50 |
| 128 | IIIGHHAYG | 292 | AVTGHHAYP | 327 | GHHAY | 297 | A2601 | −2.01 | −0.68 | 0.17 |
| 128 | IIIGHHAYG | 292 | NPGGHHAYF | 328 | GHHAY | 297 | A2601 | −2.01 | −0.68 | −0.06 |
| 128 | IIIGHHAYG | 292 | NIRGHHAYV | 329 | GHHAY | 297 | B0801 | −2.06 | −0.99 | −0.20 |
| 128 | IIIGHHAYG | 292 | VPRGHHAYL | 330 | GHHAY | 297 | B0801 | −2.05 | −0.99 | 0.28 |
| 129 | IIGHHAYGD | 293 | FTDHHAYGT | 331 | HHAYG | 298 | A0101 | −2.03 | −0.41 | −0.47 |
| 129 | IIGHHAYGD | 293 | DLQHHAYGY | 332 | HHAYG | 298 | A0101 | −2.03 | −0.41 | −0.38 |
| 129 | IIGHHAYGD | 293 | KRAHHAYGW | 333 | HHAYG | 298 | A2402 | −2.01 | −0.13 | −1.02 |
| 129 | IIGHHAYGD | 293 | RRTHHAYGW | 334 | HHAYG | 298 | A2402 | −2.01 | −0.13 | −1.21 |
| 129 | IIGHHAYGD | 293 | QGMHHAYGR | 335 | HHAYG | 298 | A6801 | −2.03 | −0.23 | −1.05 |
| 129 | IIGHHAYGD | 293 | DGIHHAYGR | 336 | HHAYG | 298 | A6801 | −2.03 | −0.23 | −1.00 |
| 129 | IIGHHAYGD | 293 | EEGHHAYGK | 337 | HHAYG | 298 | B4402 | −2.01 | −0.13 | −2.38 |
| 129 | IIGHHAYGD | 293 | FEIHHAYGT | 338 | HHAYG | 298 | B4402 | −2.01 | −0.13 | 0.46 |

NOTE:
Binding is shown in standard deviation units comparing all peptides within the protein. While actual affinity varies between proteins a value of −1 SD equates to 100-200 nanomolar

TABLE 11
Exemplars of peptides designed to provide high binding affinity for various MHC II alleles to the tumor specific T cell
exposed motifs of IDH R132H

| | | | | | | | | Affinity | Affinity | |
|---|---|---|---|---|---|---|---|---|---|---|
| | originating | SEQ ID | proposed | SEQ ID | TCEM | SEQ | | proposed | original | |
| pos | peptide | NO.: | peptide | NO.: | core | ID NO.: | Allele | peptide | peptide | polarity |
| 122 | SGWVKPIIIGHHAYG | 339 | PMKFKPIIIGHPKGN | 349 | KP~I~GH | 344 | DRB1_0401 | −2.01 | −0.68 | 0.18 |
| 122 | SGWVKPIIIGHHAYG | 339 | LLLRKPIIIGHAKRS | 350 | KP~I~GH | 344 | DRB1_0401 | −2.01 | −0.68 | 0.57 |
| 122 | SGWVKPIIIGHHAYG | 339 | IVWFKPIIIGHKRDA | 351 | KP~I~GH | 344 | DRB1_0801 | −2.01 | −0.83 | 1.06 |
| 122 | SGWVKPIIIGHHAYG | 339 | WVLWKPIIIGHKEQA | 352 | KP~I~GH | 344 | DRB1_0801 | −2.00 | −0.83 | 1.14 |
| 122 | SGWVKPIIIGHHAYG | 339 | YQPWKPIIIGHGEKH | 353 | KP~I~GH | 344 | DRB1_1101 | −2.02 | −1.31 | −0.10 |
| 122 | SGWVKPIIIGHHAYG | 339 | SMIPKPIIIGHNKWT | 354 | KP~I~GH | 344 | DRB1_1101 | −2.01 | −1.31 | 0.75 |
| 122 | SGWVKPIIIGHHAYG | 339 | NLTIKPIIIGHLTRN | 355 | KP~I~GH | 344 | DRB1_1501 | −2.02 | −1.31 | 0.79 |
| 122 | SGWVKPIIIGHHAYG | 339 | PFRIKPIIIGHRIQT | 356 | KP~I~GH | 344 | DRB1_1501 | −2.02 | −1.31 | 0.83 |
| 123 | GWVKPIIIGHHAYGD | 340 | TDLMPIIIGHHDKIH | 357 | PI~I~HH | 345 | DRB1_0401 | −2.00 | −1.49 | 0.58 |
| 123 | GWVKPIIIGHHAYGD | 340 | KGIYPIIIGHHHGQL | 358 | PI~I~HH | 345 | DRB1_0401 | −2.00 | −1.49 | 1.01 |
| 123 | GWVKPIIIGHHAYGD | 340 | LNIFPIIIGHHKNNI | 359 | PI~I~HH | 345 | DRB1_0801 | −2.01 | −0.61 | 0.54 |
| 123 | GWVKPIIIGHHAYGD | 340 | YIKQPIIIGHHAKTF | 360 | PI~I~HH | 345 | DRB1_0801 | −2.01 | −0.61 | 0.83 |
| 123 | GWVKPIIIGHHAYGD | 340 | ATFQPIIIGHHPKQP | 361 | PI~I~HH | 345 | DRB1_1101 | −2.01 | −1.14 | 0.39 |
| 123 | GWVKPIIIGHHAYGD | 340 | GWIEPIIIGHHQQNM | 362 | PI~I~HH | 345 | DRB1_1101 | −2.00 | −1.14 | 0.79 |
| 123 | GWVKPIIIGHHAYGD | 340 | YHILPIIIGHHSQNN | 363 | PI~I~HH | 345 | DRB1_1501 | −2.00 | −1.01 | 0.80 |
| 123 | GWVKPIIIGHHAYGD | 340 | GGIIPIIIGHHEDRS | 364 | PI~I~HH | 345 | DRB1_1501 | −2.00 | −1.01 | 0.34 |
| 125 | VKPIIIGHHAYGDQY | 341 | PKVIIIGHHAYAKGK | 365 | II~H~AY | 346 | DRB1_0401 | −2.02 | −1.35 | 0.14 |
| 125 | VKPIIIGHHAYGDQY | 341 | QYQIIIGHHAYEQGA | 366 | II~H~AY | 346 | DRB1_0401 | −2.01 | 1.35 | 0.15 |
| 125 | VKPIIIGHHAYGDQY | 341 | GPLIIIGHHAYKEQF | 367 | II~H~AY | 346 | DRB1_0801 | −2.01 | −1.59 | 0.93 |
| 125 | VKPIIIGHHAYGDQY | 341 | HFLMIIGHHAYEDNA | 368 | II~H~AY | 346 | DRB1_0801 | −2.01 | −1.59 | 0.72 |
| 125 | VKPIIIGHHAYGDQY | 341 | NIWAIIGHHAYWDEG | 369 | II~H~AY | 346 | DRB1_1101 | −2.01 | −1.01 | 0.84 |
| 125 | VKPIIIGHHAYGDQY | 341 | KNYMIIGHHAYYEKS | 370 | II~H~AY | 346 | DRB1_1101 | −2.00 | −1.01 | −0.40 |
| 125 | VKPIIIGHHAYGDQY | 341 | KQTRIIGHHAYILLQ | 371 | II~H~AY | 346 | DRB1_1501 | −2.02 | −0.87 | 0.55 |
| 125 | VKPIIIGHHAYGDQY | 341 | KPDIIIGHHAYKDFE | 372 | II~H~AY | 346 | DRB1_1501 | −2.01 | −0.87 | −0.44 |
| 127 | PIIIGHHAYGDQYRA | 342 | PWIFGHHAYGDKSGL | 373 | GH~A~GD | 347 | DRB1_0401 | −2.14 | −1.45 | 0.44 |
| 127 | PIIIGHHAYGDQYRA | 342 | FLILGHHAYGDNKSM | 374 | GH~A~GD | 347 | DRB1_0401 | −2.13 | −1.45 | 0.54 |
| 127 | PIIIGHHAYGDQYRA | 342 | LMFWGHHAYGDEIYF | 375 | GH~A~GD | 347 | DRB1_0801 | −1.80 | −0.83 | 1.66 |
| 127 | PIIIGHHAYGDQYRA | 342 | IFFWGHHAYGDIKKD | 376 | GH~A~GD | 347 | DRB1_0801 | −1.48 | −0.83 | 0.31 |
| 127 | PIIIGHHAYGDQYRA | 342 | AKFWGHHAYGDLSRP | 377 | GH~A~GD | 347 | DRB1_1101 | −2.05 | −1.00 | −0.32 |
| 127 | PIIIGHHAYGDQYRA | 342 | KFIFGHHAYGDYDKK | 378 | GH~A~GD | 347 | DRB1_1101 | −2.04 | −1.00 | −0.79 |
| 127 | PIIIGHHAYGDQYRA | 342 | TAYVGHHAYGDWYYI | 379 | GH~A~GD | 347 | DRB1_1501 | −2.01 | −0.76 | 0.99 |
| 127 | PIIIGHHAYGDQYRA | 342 | ELIFGHHAYGDWFLI | 380 | GH~A~GD | 347 | DRB1_1501 | −1.87 | −0.76 | 2.10 |
| 128 | IlIGHHAYGDQYRAT | 343 | KRVFHHAYGDQAITL | 381 | HH~Y~DQ | 348 | DRB1_0401 | −2.01 | −0.52 | −0.07 |
| 128 | IIIGHHAYGDQYRAT | 343 | GRLFHHAYGDQLLDL | 382 | HH~Y~DQ | 348 | DRB1_0401 | −2.01 | −0.52 | 0.52 |
| 128 | IIIGHHAYGDQYRAT | 343 | MFFYHHAYGDQIKDN | 383 | HH~Y~DQ | 348 | DRB1_0801 | −2.02 | −0.20 | −0.13 |
| 128 | IIIGHHAYGDQYRAT | 343 | LYYFHHAYGDQQATD | 384 | HH~Y~DQ | 348 | DRB1_0801 | −2.02 | −0.20 | −0.29 |
| 128 | IIIGHHAYGDQYRAT | 343 | TVLRHHAYGDQWYKS | 385 | HH~Y~DQ | 348 | DRB1_1101 | −2.02 | −0.42 | −0.67 |
| 128 | IIIGHHAYGDQYRAT | 343 | IKMLHHAYGDQLDDT | 386 | HH~Y~DQ | 348 | DRB1_1101 | −2.01 | −0.42 | −0.59 |

NOTE:
Binding is shown in standard deviation units comparing all peptides within the protein. While actual affinity varies between proteins a value of −1 SD equates to 100-200 nanomolar

### Example 7: Mutations of Histone H3.3

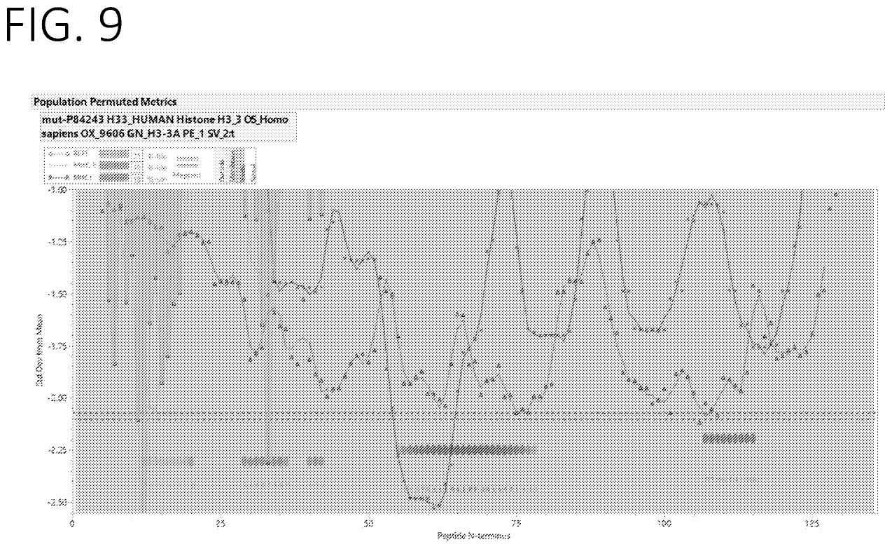

Histone H3.3 (also written as H33), is encoded by sequence P84243, and is a variant of histone 3 found in non-dividing cells. Mutations of this variant are associated with hypermethylation, instability and pathogenesis of glioma and glioblastoma. The most common mutation is referred to as K27M, although in fact it is a replacement of a lysine at position 28 in the canonical isoform 1 of the protein. Position 28 is located in a region which peptides very poor or no predicted binding to either MHC I or MHC II alleles (FIG. 9). Thus, the only feasible approach to targeting the mutation is to utilize peptides engineered to match the subject's alleles, making prior determination of HLA a requisite. Tables 12 and 13 provide examples of such bespoke peptides for an illustrative set of alleles. These examples are considered non limiting as the same approach can be applied for other alleles and multiple peptide options exist for each allele.

TABLE 12
Exemplars of peptides designed to provide high binding affinity for various MHC I alleles to the tumor specific T cell
exposed motifs of H3.3 K27M (K28M)

| | Originating | SEQ | proposed | SEQ | | SEQ | | Synthetic peptide | Natural peptide | |
|---|---|---|---|---|---|---|---|---|---|---|
| pos | peptide | ID NO.: | peptide | ID NO.: | TCEM core | ID NO.: | Allele | binding affinity | binding affinity | polarity |
| 21 | LATKAARMS | 387 | KATKAARMY | 397 | KAARM | 392 | A0101 | -2.03 | -0.13 | -1.36 |
| 21 | LATKAARMS | 387 | FAQKAARMR | 398 | KAARM | 392 | A0101 | -2.02 | -0.13 | -1.08 |
| 21 | LATKAARMS | 387 | TQRKAARMY | 399 | KAARM | 392 | B5701 | -2.01 | -0.20 | -1.83 |
| 21 | LATKAARMS | 387 | LKKKAARMY | 400 | KAARM | 392 | B5701 | -2.01 | -0.20 | -1.29 |
| 22 | ATKAARMSA | 388 | PVEAARMSR | 401 | AARMS | 393 | A0101 | -2.03 | -0.17 | -1.18 |
| 22 | ATKAARMSA | 388 | RPDAARMSR | 402 | AARMS | 393 | A0101 | -2.02 | -0.17 | -2.45 |
| 22 | ATKAARMSA | 388 | KAKAARMSL | 403 | AARMS | 393 | B0702 | -2.01 | -1.01 | -1.05 |
| 22 | ATKAARMSA | 388 | RGKAARMSA | 404 | AARMS | 393 | B0702 | -2.01 | -1.01 | -1.82 |
| 22 | ATKAARMSA | 388 | EQEAARMSY | 405 | AARMS | 393 | B1501 | -2.01 | -0.14 | -1.79 |
| 22 | ATKAARMSA | 388 | GTRAARMSY | 406 | AARMS | 393 | B1501 | -2.00 | -0.14 | -1.03 |
| 23 | TKAARMSAP | 389 | SSEARMSAR | 407 | ARMSA | 394 | A2601 | -2.05 | -0.30 | -2.29 |
| 23 | TKAARMSAP | 389 | EPDARMSAQ | 408 | ARMSA | 394 | A2601 | -2.04 | -0.30 | -2.12 |
| 23 | TKAARMSAP | 389 | KSRARMSAL | 409 | ARMSA | 394 | B0702 | -2.02 | -0.11 | -1.38 |
| 23 | TKAARMSAP | 389 | RTRARMSAV | 410 | ARMSA | 394 | B0702 | -2.01 | -0.11 | -1.40 |
| 23 | TKAARMSAP | 389 | QRDARMSAL | 411 | ARMSA | 394 | B0801 | -2.01 | -0.54 | -1.45 |
| 23 | TKAARMSAP | 389 | KPGARMSAA | 412 | ARMSA | 394 | B0801 | -2.01 | -0.54 | -1.12 |
| 24 | KAARMSAPS | 390 | RLDRMSAPQ | 413 | RMSAP | 395 | A0101 | -2.03 | -0.15 | -1.54 |
| 24 | KAARMSAPS | 390 | FQERMSAPR | 414 | RMSAP | 395 | A0101 | -2.02 | -0.15 | -1.43 |
| 24 | KAARMSAPS | 390 | KLTRMSAPT | 415 | RMSAP | 395 | A0201 | -2.02 | -0.01 | -0.78 |
| 24 | KAARMSAPS | 390 | RQIRMSAPA | 416 | RMSAP | 395 | A0201 | -2.00 | -0.01 | -0.78 |
| 24 | KAARMSAPS | 390 | NLIRMSAPR | 417 | RMSAP | 395 | A1101 | -2.02 | -0.18 | -0.17 |
| 24 | KAARMSAPS | 390 | RQYRMSAPK | 418 | RMSAF | 395 | A1101 | -2.01 | -0.18 | -2.05 |
| 24 | KAARMSAPS | 390 | RPRRMSAPD | 419 | RMSAP | 395 | B0702 | -2.02 | -0.55 | -2.53 |
| 24 | KAARMSAPS | 390 | KPSRMSAPG | 420 | RMSAP | 395 | B0702 | -2.01 | -0.55 | -1.58 |
| 24 | KAARMSAPS | 390 | QTRRMSAPA | 421 | RMSAP | 395 | B0801 | -2.02 | -0.07 | -1.68 |
| 24 | KAARMSAPS | 390 | RDRRMSAPI | 422 | RMSAP | 395 | B0801 | -2.02 | -0.07 | -1.79 |
| 24 | KAARMSAPS | 390 | DDARMSAPY | 423 | RMSAP | 395 | B1501 | -2.02 | -0.02 | -1.43 |
| 24 | KAARMSAPS | 390 | KERRMSAPF | 424 | RMSAP | 395 | B1501 | -2.02 | -0.02 | -1.73 |
| 25 | AARMSAPST | 391 | LEEMSAPSP | 425 | MSAPS | 396 | A0101 | -2.02 | -0.22 | -0.59 |
| 25 | AARMSAPST | 391 | KAAMSAPSY | 426 | MSAPS | 396 | A0101 | -2.01 | -0.22 | -0.35 |
| 25 | AARMSAPST | 391 | DTGMSAPSR | 427 | MSAPS | 396 | A2601 | -2.04 | -0.18 | -1.67 |
| 25 | AARMSAPST | 391 | ESTMSAPSR | 428 | MSAPS | 396 | A2601 | -2.03 | -0.18 | -1.84 |
| 25 | AARMSAPST | 391 | RPSMSAPSS | 429 | MSAPS | 396 | B0702 | -2.02 | -1.65 | -1.34 |
| 25 | AARMSAPST | 391 | DPKMSAPSA | 430 | MSAPS | 396 | B0702 | -2.01 | -1.65 | -1.39 |
| 25 | AARMSAPST | 391 | KTKMSAPSM | 431 | MSAPS | 396 | B1501 | -2.03 | -0.04 | -1.23 |
| 25 | AARMSAPST | 391 | KAQMSAPSY | 432 | MSAPS | 396 | B1501 | -2.03 | -0.04 | -0.87 |
| 25 | AARMSAPST | 391 | ASVMSAPSF | 433 | MSAPS | 396 | B5701 | -2.09 | -0.15 | 1.06 |
| 25 | AARMSAPST | 391 | RAQMSAPSY | 434 | MSAPS | 396 | B5701 | -2.04 | -0.15 | -0.83 |

NOTE:
Binding is shown in standard deviation units comparing all peptides within the protein. While actual affinity varies between proteins a value of −1 SD equates to 100-200 nanomolar

TABLE 13
Exemplars of peptides designed to provide high binding affinity for various MHC II alleles to
the tumor specific T cell exposed motifs of H3.3 K27M (K28M)

| | | | | | | | Natural | Synthetic | |
|---|---|---|---|---|---|---|---|---|---|
| | | SEQ | | SEQ | | SEQ | peptide | peptide | |
| | Originating | ID | | ID | TCEM | ID | binding | binding | |
| pos | peptide | NO.: | proposed peptide | NO.: | core | NO.: | affinity | affinity | polarity |
| 18 | RKQLATKAARMSAPS | 435 | QLLIATKAARMSVDV | 447 | AT~A~RM | 441 | −0.12 | −1.90 | 0.58 |
| 18 | RKQLATKAARMSAPS | 435 | TMIIATKAARMFARK | 448 | AT~A~RM | 441 | −0.12 | −1.71 | 0.19 |
| 18 | RKQLATKAARMSAPS | 435 | DRLRATKAARMGEAA | 449 | AT~A~RM | 441 | −0.56 | −2.02 | −1.68 |
| 18 | RKQLATKAARMSAPS | 435 | GRITATKAARMEMQI | 450 | AT~A~RM | 441 | −0.56 | −2.08 | −0.59 |
| 19 | KQLATKAARMSAPST | 436 | KFLRTKAARMSTYDA | 451 | TK~A~MS | 442 | −0.53 | −2.01 | −0.99 |
| 19 | KQLATKAARMSAPST | 436 | PKFRTKAARMSTFPR | 452 | TK~A~MS | 442 | −0.53 | −2.03 | −1.19 |
| 21 | LATKAARMSAPSTGG | 437 | AKKYAARMSAPTRKG | 453 | AA~M~AP | 443 | −2.35 | −2.01 | −1.86 |
| 21 | LATKAARMSAPSTGG | 437 | KRATAARMSAPCATH | 454 | AA~M~AP | 443 | −2.35 | −2.01 | −1.17 |
| 23 | TKAARMSAPSTGGVK | 438 | RARLRMSAPSTIAQG | 455 | RM~A~ST | 444 | −0.35 | −2.01 | −0.72 |
| 23 | TKAARMSAPSTGGVK | 438 | TRAFRMSAPSTLADQ | 456 | RM~A~ST | 444 | −0.35 | −2.01 | −0.71 |
| 24 | KAARMSAPSTGGVKK | 439 | EAILMSAPSTGIASE | 457 | MS~P~TG | 445 | −0.41 | −2.01 | 0.31 |
| 24 | KAARMSAPSTGGVKK | 439 | RPRIMSAPSTGLETA | 458 | MS~P~TG | 445 | −0.41 | −2.01 | −0.56 |
| 24 | KAARMSAPSTGGVKK | 439 | AGRRMSAPSTGQGYC | 459 | MS~P~TG | 445 | −0.98 | −2.02 | −1.02 |
| 24 | KAARMSAPSTGGVKK | 439 | KRMRMSAPSTGGDLI | 460 | MS~P~TG | 445 | −0.98 | −2.04 | −0.76 |
| 25 | AARMSAPSTGGVKKP | 440 | ALFFSAPSTGGVRQQ | 461 | SA~S~GG | 446 | −0.30 | −1.91 | 0.31 |
| 25 | AARMSAPSTGGVKKP | 440 | FIFLSAPSTGGAKLQ | 462 | SA~S~GG | 446 | −0.30 | −1.96 | 1.11 |

NOTE:
Binding is shown in standard deviation units comparing all peptides within the protein. While actual affinity varies between proteins a value of −1 SD equates to 100-200 nanomolar

### Example 8: BRAF V600E

BRAF (Serine/threonine-protein kinase B-raf) exemplified by sequence P15056 is one of the most commonly mutated proteins in cancer. BRAF is thought to function in regulating the MAP kinase/ERKs signaling pathway, which affects cell division, differentiation, and secretion. BRAF mutations have been associated with various cancers, including non-Hodgkin lymphoma, colorectal cancer, malignant melanoma, thyroid carcinoma, non-small cell lung carcinoma, and adenocarcinoma of the lung. There are several particularly common mutations in BRAF but the most common is V600E. Mutation V600M, G and K are less common.

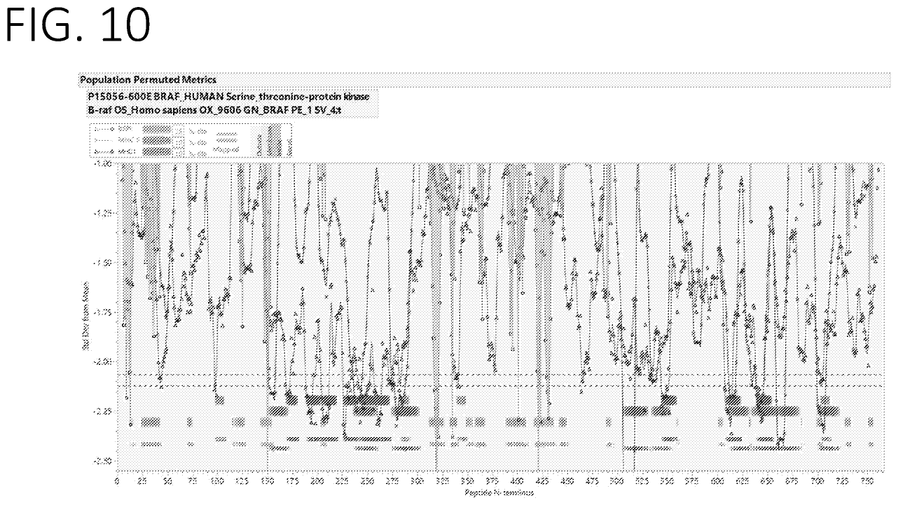

FIG. 10 provides and overview of MHC binding regions in BRAF V600E. Natural binding of MHC I A alleles to the T cell exposed motif which would be tumor specific in the V600E mutation is very sparse, with no alleles having an optimum binding and very even moderate binding. Furthermore the adjacent peptides on the C terminal side of the mutation which would place the mutant E in a pocket position, or “out of frame” have an extremely high binding affinity for both A and B alleles, which would tend to favor binding preferentially in that position, hiding the mutant amino acid. The same is true for some MHC I A and B alleles for the V600M mutant. Thus the desirable approach is to create peptides with the mutant amino acid in the T cell exposed position and with modified amino acids in the groove exposed positions binding to stimulate T cell clones that can target the tumor specifically. Table 14 provides examples for exemplar selected alleles, using the method described in Example 10. As many different peptides can be designed with flanking regions that produce the desired binding affinity and solubility, these examples shown are provided as illustrative but non-limiting examples. Table 15 includes examples of MHC II DRB binding peptides which can provide CD4. It is noted that the naturally occurring peptide GSHQFEQLSGSILWM (SEQ ID NO.: 510) provides a suitable predicted binding affinity for DRB1_0701 and could be used in this form. In addition naturally occurring adjacent peptides have suitable natural binding affinity and could provide CD4+ help, albeit not embodying the tumor specific amino acid motif. This short sequence of peptides comprise SGSILWMAPEVIRMQ (SEQ ID NO.: 535), GSILWMAPEVIRMQD (SEQ ID NO.: 536) and SILWMAPEVIRMQDK (SEQ ID NO.: 537).

TABLE 14
Exemplars of peptides designed to provide high binding affinity for various MHC I alleles to
the tumor specific T cell exposed motifs of BRAF V600E

| | | | | | | | | | | Predicted | |
|---|---|---|---|---|---|---|---|---|---|---|---|
| | | | | | | | | | Predicted | affinity | |
| | | Origin- | SEQ | | SEQ | | SEQ | | affinity | origin- | |
| | | ating | ID | proposed | ID | TCEM | ID | | proposed | ating | pol- |
| gi | pos | peptide | NO.: | peptide | NO.: | core | NO.: | Allele | peptide | peptide | arity |
| P15056-600E | 604 | WSGSHQFEQ | 463 | KLVSHQFEY | 473 | SHQFE | 468 | A1101 | −2.02 | −0.39 | −0.04 |
| P15056-600E | 604 | WSGSHQFEQ | 463 | SIDSHQFER | 474 | SHQFE | 468 | A1101 | −2.02 | −0.39 | −1.78 |
| P15056-600E | 604 | WSGSHQFEQ | 463 | AAASHQFEF | 475 | SHQFE | 468 | B1501 | −2.11 | −0.17 | 0.18 |
| P15056-600E | 604 | WSGSHQFEQ | 463 | SSKSHQFEF | 476 | SHQFE | 468 | B1501 | −2.00 | −0.17 | −1.39 |
| P15056-600E | 604 | WSGSHQFEQ | 463 | GAISHQFER | 477 | SHQFE | 468 | B5701 | −2.02 | −0.54 | −0.82 |
| P15056-600E | 604 | WSGSHQFEQ | 463 | HSPSHQFEL | 478 | SHQFE | 468 | B5701 | −2.01 | −0.54 | −0.70 |
| P15056-600E | 605 | SGSHQFEQL | 464 | KLYHQFEQS | 479 | HQFEQ | 469 | A0201 | −2.03 | −0.13 | −1.02 |
| P15056-600E | 605 | SGSHQFEQL | 464 | AMDHQFEQA | 480 | HQFEQ | 469 | A0201 | −2.02 | −0.13 | −1.05 |
| P15056-600E | 605 | SGSHQFEQL | 464 | FPQHQFEQP | 481 | HQFEQ | 469 | B0702 | −1.98 | −0.48 | −0.65 |
| P15056-600E | 605 | SGSHQFEQL | 464 | IPLHQFEQY | 482 | HQFEQ | 469 | B0702 | −2.04 | −0.48 | 0.75 |
| P15056-600E | 605 | SGSHQFEQL | 464 | SSTHQFEQF | 483 | HQFEQ | 469 | B1501 | −2.05 | −0.45 | −0.95 |
| P15056-600E | 605 | SGSHQFEQL | 464 | KVPHQFEQY | 484 | HQFEQ | 469 | B1501 | −2.00 | −0.45 | −0.93 |
| P15056-600E | 605 | SGSHQFEQL | 464 | LSSHQFEQF | 485 | HQFEQ | 469 | A2601 | −2.01 | −0.83 | −0.06 |
| P15056-600E | 605 | SGSHQFEQL | 464 | DSFHQFEQL | 486 | HQFEQ | 469 | A2601 | −2.01 | −0.83 | −0.43 |
| P15056-600E | 605 | SGSHQFEQL | 464 | PIHHQFEQC | 487 | HQFEQ | 469 | B0801 | −2.07 | −1.43 | −0.31 |
| P15056-600E | 605 | SGSHQFEQL | 464 | ELAHQFEQG | 488 | HQFEQ | 469 | B0801 | −2.04 | −1.43 | −0.91 |
| P15056-600E | 605 | SGSHQFEQL | 464 | LALHQFEQM | 489 | HQFEQ | 469 | B5701 | −2.03 | −0.96 | −1.00 |
| P15056-600E | 605 | SGSHQFEQL | 464 | GEEHQFEQW | 490 | HQFEQ | 469 | B5701 | −2.03 | −0.96 | −1.72 |
| P15056-600E | 606 | GSHQFEQLS | 465 | SAKQFEQLR | 491 | QFEQL | 470 | A1101 | −2.03 | −0.32 | −1.64 |
| P15056-600E | 606 | GSHQFEQLS | 465 | GEAQFEQLK | 492 | QFEQL | 470 | A1101 | −2.02 | −0.32 | −1.36 |
| P15056-600E | 606 | GSHQFEQLS | 465 | EVKQFEQLK | 493 | QFEQL | 470 | A3001 | −2.02 | −1.00 | −1.57 |
| P15056-600E | 606 | GSHQFEQLS | 465 | RVDQFEQLG | 494 | QFEQL | 470 | A3001 | −2.02 | −1.00 | −0.91 |
| P15056-600E | 607 | SHQFEQLSG | 466 | DFKFEQLSA | 495 | FEQLS | 471 | A3001 | −2.02 | −0.31 | −0.37 |
| P15056-600E | 607 | SHQFEQLSG | 466 | GGRFEQLSN | 496 | FEQLS | 471 | A3001 | −2.02 | −0.31 | −1.19 |
| P15056-600E | 607 | SHQFEQLSG | 466 | YKLFEQLSI | 497 | FEQLS | 471 | B0801 | −2.02 | −0.69 | 1.18 |
| P15056-600E | 607 | SHQFEQLSG | 466 | YLGFEQLSA | 498 | FEQLS | 471 | B0801 | −2.04 | −0.69 | 1.18 |
| P15056-600E | 608 | HQFEQLSGS | 467 | IMHEQLSGS | 499 | EQLSG | 472 | A0201 | −2.02 | −0.48 | −0.10 |
| P15056-600E | 608 | HQFEQLSGS | 467 | KIEEQLSGL | 500 | EQLSG | 472 | A0201 | −2.02 | −0.48 | −0.62 |
| P15056-600E | 608 | HQFEQLSGS | 467 | DEAEQLSGF | 501 | EQLSG | 472 | B1501 | −2.04 | −0.77 | −1.15 |
| P15056-600E | 608 | HQFEQLSGS | 467 | FYLEQLSGF | 502 | EQLSG | 472 | B1501 | −2.04 | −0.77 | 1.89 |
| P15056-600E | 608 | HQFEQLSGS | 467 | TTKEQLSGS | 503 | EQLSG | 472 | A3001 | −2.02 | −0.53 | −2.07 |
| P15056-600E | 608 | HQFEQLSGS | 467 | RSQEQLSGA | 504 | EQLSG | 472 | A3001 | −2.01 | −0.53 | −2.08 |
| P15056-600E | 608 | HQFEQLSGS | 467 | EAVEQLSGS | 505 | EQLSG | 472 | A2601 | −2.01 | −0.84 | −1.04 |
| P15056-600E | 608 | HQFEQLSGS | 467 | NQREQLSGY | 506 | EQLSG | 472 | A2601 | −2.01 | −0.84 | −2.06 |
| P15056-600E | 608 | HQFEQLSGS | 467 | SLGEQLSGA | 507 | EQLSG | 472 | B0801 | −2.08 | −0.97 | −0.29 |
| P15056-600E | 608 | HQFEQLSGS | 467 | MNVEQLSGL | 508 | EQLSG | 472 | B0801 | −2.09 | −0.97 | 0.52 |

NOTE:
Binding is shown in standard deviation units comparing all peptides within the protein. While actual affinity varies between proteins a value of −1 SD equates to 100-200 nanomolar

TABLE 15
Exemplars of peptides designed to provide high binding affinity for various MHC II alleles to the tumor specific T cell
exposed motifs of BRAF V600E

| | | | | | | | | | Predicted | Predicted | |
|---|---|---|---|---|---|---|---|---|---|---|---|
| | | | SEQ | | SEQ | | SEQ | | affinity | affinity | |
| | | originating | ID | proposed | ID | TCEM | ID | | proposed | originating | |
| gi | pos | peptide | NO.: | peptide | NO.: | core | NO.: | Allele | peptide | peptide | polarity |
| P15056-600E | 604 | WSGSHQFEQLSGSIL | 509 | IHKFHQFEQLSPPSQ | 515 | HQ~E~LS | 512 | DRB1_0401 | 2.07 | −1.02 | 0.47 |
| P15056-600E | 604 | WSGSHQFEQLSGSIL | 509 | PHFIHQFEQLSGFAM | 516 | HQ~E~LS | 512 | DRB1_0401 | 2.04 | −1.02 | 1.17 |
| P15056-600E | 604 | WSGSHQFEQLSGSIL | 509 | YRYYHQFEQLSMPQV | 517 | HQ~E~LS | 512 | DRB1_0701 | −2.07 | −0.41 | 0.09 |
| P15056-600E | 604 | WSGSHQFEQLSGSIL | 509 | HMMFHQFEQLSKLQL | 518 | HQ~E~LS | 512 | DRB1_0701 | −2.01 | −0.41 | 0.69 |
| P15056-600E | 604 | WSGSHQFEQLSGSIL | 509 | WLPYHQFEQLSSPIP | 519 | HQ~E~LS | 512 | DRB1_1501 | −2.05 | −0.14 | 0.91 |
| P15056-600E | 604 | WSGSHQFEQLSGSIL | 509 | FFECHQFEQLSWLSV | 520 | HQ~E~LS | 512 | DRB1_1501 | −2.07 | −0.14 | 1.34 |
| P15056-600E | 606 | GSHQFEQLSGSILWM | 510 | SSWPFEQLSGSTGDN | 521 | FE~L~GS | 513 | DRB1_0401 | −2.08 | −0.90 | −0.94 |
| P15056-600E | 606 | GSHQFEQLSGSILWM | 510 | GPGMFEQLSGSTELF | 522 | FE~L~GS | 513 | DRB1_0401 | −2.07 | −0.90 | 0.49 |
| P15056-600E | 606 | GSHQFEQLSGSILWM | 510 | LEVFFEQLSGSSLAN | 523 | FE~L~GS | 513 | DRB1_0701 | −2.14 | −2.1 | 0.75 |
| P15056-600E | 606 | GSHQFEQLSGSILWM | 510 | RRILFEQLSGSCVGL | 524 | FE~L~GS | 513 | DRB1_0701 | −2.08 | −2.11 | 0.76 |
| P15056-600E | 606 | GSHQFEQLSGSILWM | 510 | WYDIFEQLSGSNAPS | 525 | FE~L~GS | 513 | DRB1-1101 | −2.11 | −0.44 | 0.04 |
| P15056-600E | 606 | GSHQFEQLSGSILWM | 510 | LVFIFEQLSGSNWRA | 526 | FE~L~GS | 513 | DRB1-1101 | −2.09 | −0.44 | 1.27 |
| P15056-600E | 606 | GSHQFEQLSGSILWM | 510 | RGAYFEQLSGSVMFS | 527 | FE~L~GS | 513 | DRB1_1501 | −2.00 | −1.65 | 0.52 |
| P15056-600E | 606 | GSHQFEQLSGSILWM | 510 | FYFYFEQLSGSLDSG | 528 | FE~L~GS | 513 | DRB1_1501 | −2.06 | −1.65 | 0.98 |
| P15056-600E | 607 | SHQFEQLSGSILWMA | 511 | PPLFEQLSGSIPICM | 529 | EQ~S~SI | 514 | DRB1_0401 | −2.01 | −1.19 | 1.61 |
| P15056-600E | 607 | SHQFEQLSGSILWMA | 511 | YPRFEQLSGSIKIIG | 530 | EQ~S~SI | 514 | DRB1_0401 | −2.01 | −1.19 | 0.45 |
| P15056-600E | 607 | SHQFEQLSGSILWMA | 511 | PTGQEQLSGSIIVIF | 531 | EQ~S~SI | 514 | DRB1_0701 | −2.11 | −1.83 | 1.11 |
| P15056-600E | 607 | SHQFEQLSGSILWMA | 511 | PKVLEQLSGSIFVGF | 532 | EQ~S~SI | 514 | DRB1_0701 | 1.99 | −1.83 | 1.37 |
| P15056-600E | 607 | SHQFEQLSGSILWMA | 511 | IFHFEQLSGSIFILI | 533 | EQ~S~SI | 514 | DRB1_1501 | −2.02 | −0.40 | 2.87 |
| P15056-600E | 607 | SHQFEQLSGSILWMA | 511 | RFRFEQLSGSILSLT | 534 | EQ~S~SI | 514 | DRB1_1501 | −2.07 | −0.40 | 0.56 |

NOTE:
Binding is shown in standard deviation units comparing all peptides within the protein. While actual affinity varies between proteins a value of −1 SD equates to 100-200 nanomolar

### Example 9: Selection of Mutant Peptides and Generation of Better Binding Peptides

In order for a T cell to differentially target a tumor cell expressing a mutated protein, the mutated amino acid has to be located in a position “visible” or exposed to the T cell receptor and not hidden in the pocket or groove exposed positions that determine binding. A first step in designing a multi peptide vaccine or stimulant panel is therefore to identify those peptide positions which expose the mutated amino acid. For MHC I this means the mutant amino acid must be at positions 4, 5, 6, 7 or 8 of a 9-mer peptide and for MHC II at positions 2, 3, 5, 7, 8 of the 9-mer core of a 15 mer. This identifies TCEM IIA; TCEM IIB positions are at −1, 3, 5, 7, 8. We first calculated the predicted binding affinity of all sequential peptide positions in the mutant protein and then selected those peptides with relevant TCEM comprising mutated amino acids.

A T cell is only able to target a TCEM if that motif is presented in the host from the naturally occurring mutant peptide. Mutant TCEM that lie in peptides that are extremely unlikely to ever be presented are thus poor targets. The TCEM are therefore filtered to identify those which have some likelihood of exposure in the host, limiting to those whose predicted binding affinity is greater than the mean for the native protein. This is not an absolute requirement but maximizes the potential for a successful targeting.

For each of the selected peptides comprising a mutant TCEM, a bank of peptides is generated by varying the flanking amino acids and recalculating the new binding affinity for each allele of interest. For a 9-mer with a pentamer exposed TCEM, this implies up to 160,000 (204) different peptides could be generated, each with a different binding affinity. For practical purposes a bank of 1000 or up to 15,000 peptides is usually sufficient to provide peptides within the range of binding affinity desired. For MHC II in preferred embodiments only those amino acids outside the core 9 mer peptide comprising the TCEM are varied, as the intercalated amino acids which are in pocket (groove exposed) positions affect binding but may also influence the positioning of the exposed amino acids.

A further practical consideration is solubility of the peptide. A score was generated based on the polarity of the constituent amino acids and only peptides likely to be soluble were put forward as candidates. Sufficient peptides can be generated to prevent this from becoming a limitation. Furthermore, peptides in which cysteines are placed in the groove exposed motifs are avoided wherever possible to minimize cross linking during formulation.

For a group of 5 proteins each with one mutation and a patient with 4 known alleles therefore a maximum number of allele TCEM combinations is 5 TCEM×5 proteins×4 alleles or 100 possible ways to stimulate T cells which will uniquely target those proteins. This is then reduced by the TCEM of low probability of natural presentation.

The development of vaccines and stimulants for dendritic cells and T cells in vitro to comprise multiple peptides with a selected desired affinity for the patient's alleles builds on methods previously described to precisely predict MHC binding, identify and analyze T cell exposed motifs and generate peptides with altered binding affinity (See PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. Nos. 10,706,955 and 10,755,801, all of which are incorporated herein by reference in their entirety).

### Example 10: Selection of Personalized Simulated Peptides

The process described in Example 9 generates a selection of peptides of different binding affinity for each combination of mutant-containing-TCEM and patient allele. Peptides are then selected which have a desired predicted binding affinity. As peptides of many different binding affinities are provided, the desired affinity may be selected. In the subsequent examples we have elected to focus on peptides with predicted binding affinity at about 2 standard deviations below the mean of the protein, placing them at about the 95^{th} percentile; i.e. the top 5% binders, but not higher, because very high affinity peptide may lead to immunosuppression or exhaustion.

Peptides may be selected to use in groups that target the maximum number of combinations of allele and TCEM in any one application. A desired aspect is to ensure not all peptides administered at any one time as a multi-neoepitope vaccine target the same allele, thus competing with each other for space in MHC and presentation. When dendritic cells and T cells are targeted in vitro it may be desirable to provide as many combinations as possible.

### Example 11: Reference to Human Proteome to Identify Potential Adverse Reactions

To identify potential off-target effects of the T cells stimulated by the peptides designed to generate targeting of cancer mutations, the TCEM are compared with those in the human proteome to identify relevant matches. The entire human proteome, comprising over 88,000 proteins (including all known isoforms of each protein), was pre-analyzed to determine the binding affinity of each peptide in each protein for all MHC alleles. The TCEM comprised in the peptides selected for each cancer patient, selected as described in Example 10 are assembled into a “call list”. The human proteome reference database is searched for all TCEM on the patient call list; a subset of proteins with matching TCEM is assembled. The peptides in this subset which contain the TCEM on the call list are then examined to determine if the TCEM would be likely to be presented in the MHC corresponding to that subject's alleles. If the proteome peptide comprising the TCEM of interest is predicted to bind to any one of the patient's known alleles with an affinity <1 SD below the mean for the protein, the protein is included in an advisory list. The list is curated to remove duplicates and references to any protein fragments catalogued in UniProt (on the world wide web at uniprot.org). Individual proteins may be reviewed in UniProt and elsewhere to determine if there is evidence of pathologies arising from deficiencies or mutations in the protein. Instances in which a protein of immediate concern is targeted are flagged with a “caution” and excluded from the proposed peptides encoded in a vaccine or in vitro cell stimulation. Examples include, but are not limited to, coagulation factors, neurotransmitters, complement, other proteins with known essential and non-redundant functions. Decision on off-targeting of proteins in the advisory list may be based on a risk-benefit analysis of the subject's condition but access to such a list allows the oncologist to make an informed decision. The most complete typing of a subject's alleles enables a more complete assessment of potential off-targets. Notably, as the relevance of each target will depend on its presentation as a result of the MHC binding of the peptide in which the TCEM occurs, identifying the potential off-target impacts is as personalized as the design of the peptide array for that cancer patient.

### Example 12: Method to Determine the RNA Fraction

Bulk RNA transcript enumeration is carried out using a bioinformatic process that has been designed to tally transcription of different genes. The resulting data is expressed as the FPKM (fragments per kilobase per million total reads) that normalizes the metric for both the length of the transcribed coding region and the number of total reads in the bulk sample detected by the sequencing machine. The bioinformatic software used for transcript enumeration (Magic-BLAST from NCBI) has been designed to assess gene expression and as such is not directly capable of measuring the frequency of potentially mutated codons within the transcripts. In order to compute the mutant frequency in the mRNA transcripts it is necessary to separately enumerate the normal and mutant transcripts. This is achieved by creating a version of the SAM (sequence alignment map) file of the RNA sequences with a bioinformatic software that modifies the cigar (compact idiosyncratic gapped alignment report) strings that map the alignments of the (missing) intronic sequences in the mRNA. Once this modified SAM file is created it can be processed with the standard mutation detection tool, such as mutect 2 [62] that provides the differential mutant and normal read tallies. The ratios of these read tallies are thus the mutant and normal frequency of the allele in the mRNA transcripts. If both parental chromosomes are being expressed equally then the frequency of the mutant and normal allele in the RNA will correlate with the frequency in the DNA. Allele specific differences in expression will give rise to poor correlations. In the extreme, where there is highly differential expression of the parental chromosomes, the mutant may be the only one expressed or may not be expressed at all compared to the normal.

Therefore, in preferred embodiments, the RNA fraction comprising the mutant amino acid is compared to the tumor DNA tumor fraction encoding the gene mutation. In some embodiments tumor specific mutations which can be targeted by T cells are selected from those in which the RNA/DNA ratio exceeds 10%.
1. Hanahan D, Weinberg R A. Hallmarks of cancer: the next generation. Cell. 2011; 144(5):646-74. Epub 2011/03/08. doi: 10.1016/j.cell.2011.02.013. PubMed PMID: 21376230.
2. Chen D S, Mellman I. Elements of cancer immunity and the cancer-immune set point. Nature. 2017; 541(7637):321-30. Epub 2017/01/20. doi: 10.1038/nature21349. PubMed PMID: 28102259.
3. Daubon T, Hemadou, A., Romero-Garmendia, I., Saleh, M. Glioblastoma Immune Landscape and the Potential of New Immunotherapies. Frontiers in immunology. 2020; 11:Article 585616. doi: doi: 10.3389/fimmu.2020.585616.
4. Bajwa R, Cheema A, Khan T, Amirpour A, Paul A, Chaughtai S, et al. Adverse Effects of Immune Checkpoint Inhibitors (Programmed Death-1 Inhibitors and Cytotoxic T-Lymphocyte-Associated Protein-4 Inhibitors): Results of a Retrospective Study. J Clin Med Res. 2019; 11(4):225-36. Epub 2019/04/03. doi: 10.14740/jocmr3750. PubMed PMID: 30937112; PubMed Central PMCID: PMCPMC6436564.
5. Gubin M M, Schreiber R D. CANCER. The odds of immunotherapy success. Science. 2015; 350(6257):158-9. Epub 2015/10/10. doi: 10.1126/science.aad4140. PubMed PMID: 26450194.
6. Lawrence M S, Stojanov P, Polak P, Kryukov G V, Cibulskis K, Sivachenko A, et al. Mutational heterogeneity in cancer and the search for new cancer-associated genes. Nature. 2013; 499(7457):214-8. Epub 2013/06/19. doi: 10.1038/nature12213. PubMed PMID: 23770567; PubMed Central PMCID: PMCPMC3919509.
7. Zander R, Schauder D, Xin G, Nguyen C, Wu X, Zajac A, et al. CD4(+) T Cell Help Is Required for the Formation of a Cytolytic CD8(+) T Cell Subset that Protects against Chronic Infection and Cancer. Immunity. 2019; 51(6):1028-42 e4. Epub 2019/12/08. doi: 10.1016/j.immuni.2019.10.009. PubMed PMID: 31810883; PubMed Central PMCID: PMCPMC6929322.
8. An Z, Aksoy O, Zheng T, Fan Q W, Weiss W A. Epidermal growth factor receptor and EGFRvIII in glioblastoma: signaling pathways and targeted therapies. Oncogene. 2018; 37(12):1561-75. Epub 2018/01/13. doi: 10.1038/s41388-017-0045-7. PubMed PMID: 29321659; PubMed Central PMCID: PMCPMC5860944.
9. Swartz A M, Li Q J, Sampson J H. Rindopepimut: a promising immunotherapeutic for the treatment of glioblastoma multiforme. Immunotherapy. 2014; 6(6):679-90. Epub 2014/09/05. doi: 10.2217/imt.14.21. PubMed PMID: 25186601; PubMed Central PMCID: PMCPMC4524671.
10. Mueller S, Taitt J M, Villanueva-Meyer J E, Bonner E R, Nejo T, Lulla R R, et al. Mass cytometry detects H3.3K27M-specific vaccine responses in diffuse midline glioma. J Clin Invest. 2020. Epub 2020/08/21. doi: 10.1172/JCI140378. PubMed PMID: 32817593.
11. Ochs K, Ott M, Bunse T, Sahm F, Bunse L, Deumelandt K, et al. K27M-mutant histone-3 as a novel target for glioma immunotherapy. Oncoimmunology. 2017; 6(7):e1328340. Epub 2017/08/16. doi: 10.1080/2162402X.2017.1328340. PubMed PMID: 28811969; PubMed Central PMCID: PMCPMC5543817.
12. Turcan S, Rohle D, Goenka A, Walsh L A, Fang F, Yilmaz E, et al. IDH1 mutation is sufficient to establish the glioma hypermethylator phenotype. Nature. 2012; 483(7390):479-83. Epub 2012/02/22. doi: 10.1038/nature10866. PubMed PMID: 22343889; PubMed Central PMCID: PMCPMC3351699.
13. Schumacher T, Bunse L, Pusch S, Sahm F, Wiestler B, Quandt J, et al. A vaccine targeting mutant IDH1 induces antitumour immunity. Nature. 2014; 512(7514):324-7. Epub 2014/07/22. doi: 10.1038/nature13387. PubMed PMID: 25043048.
14. Schumacher T, Bunse L, Wick W, Platten M. Mutant IDH1: An immunotherapeutic target in tumors. Oncoimmunology. 2014; 3(12):e974392. Epub 2015/05/13. doi: 10.4161/2162402X.2014.974392. PubMed PMID: 25964867; PubMed Central PMCID: PMCPMC4353168.
15. Ryall S, Zapotocky M, Fukuoka K, Nobre L, Guerreiro Stucklin A, Bennett J, et al. Integrated Molecular and Clinical Analysis of 1,000 Pediatric Low-Grade Gliomas. Cancer Cell. 2020; 37(4):569-83 e5. Epub 2020/04/15. doi: 10.1016/j.ccell.2020.03.011. PubMed PMID: 32289278; PubMed Central PMCID: PMCPMC7169997.
16. Cheever M A, Allison J P, Ferris A S, Finn O J, Hastings B M, Hecht T T, et al. The prioritization of cancer antigens: a national cancer institute pilot project for the acceleration of translational research. Clin Cancer Res. 2009; 15(17):5323-37. Epub 2009/09/03. doi: 10.1158/1078-0432.CCR-09-0737. PubMed PMID: 19723653; PubMed Central PMCID: PMCPMC5779623.
17. Fournier P, Schirrmacher V. Randomized clinical studies of anti-tumor vaccination: state of the art in 2008. Expert review of vaccines. 2009; 8(1):51-66. Epub 2008/12/20. doi: 10.1586/14760584.8.1.51. PubMed PMID: 19093773.
18. Klebanoff C A, Acquavella N, Yu Z, Restifo N P. Therapeutic cancer vaccines: are we there yet? Immunol Rev. 2011; 239(1):27-44. Epub 2011/01/05. doi: 10.1111/j.1600-065X.2010.00979.x. PubMed PMID: 21198663; PubMed Central PMCID: PMCPMC3075547.
19. Schreiber T H, Raez L, Rosenblatt J D, Podack E R. Tumor immunogenicity and responsiveness to cancer vaccine therapy: the state of the art. Seminars in immunology. 2010; 22(3):105-12. Epub 2010/03/17. doi: 10.1016/j.smim.2010.02.001. PubMed PMID: 20226686; PubMed Central PMCID: PMCPMC2884069.
20. Hazama S, Nakamura Y, Takenouchi H, Suzuki N, Tsunedomi R, Inoue Y, et al. A phase I study of combination vaccine treatment of five therapeutic epitope-peptides for metastatic colorectal cancer; safety, immunological response, and clinical outcome. Journal of translational medicine. 2014; 12:63. Epub 2014/03/13. doi: 10.1186/1479-5876-12-63. PubMed PMID: 24612787; PubMed Central PMCID: PMCPMC4007571.
21. Tamura R, Fujioka M, Morimoto Y, Ohara K, Kosugi K, Oishi Y, et al. A VEGF receptor vaccine demonstrates preliminary efficacy in neurofibromatosis type 2. Nature communications. 2019; 10(1):5758. Epub 2019/12/19. doi: 10.1038/s41467-019-13640-1. PubMed PMID: 31848332; PubMed Central PMCID: PMCPMC6917794.
22. Bright R K, Bright J D, Byrne J A. Overexpressed oncogenic tumor-self antigens. Human vaccines & immunotherapeutics. 2014; 10(11):3297-305. Epub 2014/12/09. doi: 10.4161/hv.29475. PubMed PMID: 25483660; PubMed Central PMCID: PMCPMC4514139.
23. Zhang W, Lu X, Cui P, Piao C, Xiao M, Liu X, et al. Phase I/11 clinical trial of a Wilms' tumor 1-targeted dendritic cell vaccination-based immunotherapy in patients with advanced cancer. Cancer Immunol Immunother. 2019; 68(1):121-30. Epub 2018/10/12. doi: 10.1007/s00262-018-2257-2. PubMed PMID: 30306202.
24. Yoshimura K, Minami T, Nozawa M, Uemura H. Phase I clinical trial of human vascular endothelial growth factor receptor 1 peptide vaccines for patients with metastatic renal cell carcinoma. Br J Cancer. 2013; 108(6):1260-6. Epub 2013/03/09. doi: 10.1038/bjc.2013.90. PubMed PMID: 23470466; PubMed Central PMCID: PMCPMC3619266.
25. Schmitz-Winnenthal F H, Hohmann N, Niethammer A G, Friedrich T, Lubenau H, Springer M, et al. Anti-angiogenic activity of VXM01, an oral T-cell vaccine against VEGF receptor 2, in patients with advanced pancreatic cancer: A randomized, placebo-controlled, phase 1 trial. Oncoimmunology. 2015; 4(4):e1001217. Epub 2015/07/03. doi: 10.1080/2162402X.2014.1001217. PubMed PMID: 26137397; PubMed Central PMCID: PMCPMC4485742.
26. Musaelyan A, Lapin S, Nazarov V, Tkachenko O, Gilburd B, Mazing A, et al. Vimentin as antigenic target in autoimmunity: A comprehensive review. Autoimmunity reviews. 2018; 17(9):926-34. Epub 2018/07/17. doi: 10.1016/j.autrev.2018.04.004. PubMed PMID: 30009963.
27. Babic I, Kesari S, Glassy M C. A Binding Potency Assay for Pritumumab and Ecto-Domain Vimentin. Methods Mol Biol. 2019; 1904:401-15. Epub 2018/12/13. doi: 10.1007/978-1-4939-8958-4_19. PubMed PMID: 30539482.
28. Babic I, Nurmemmedov E, Yenugonda V M, Juarez T, Nomura N, Pingle S C, et al. Pritumumab, the first therapeutic antibody for glioma patients. Hum Antibodies. 2018; 26(2):95-101. Epub 2017/10/19. doi: 10.3233/HAB-170326. PubMed PMID: 29036806.
29. Hagiwara H, Aotsuka Y, Yamamoto Y, Miyahara J, Mitoh Y. Determination of the antigen/epitope that is recognized by human monoclonal antibody CLN-IgG. Hum Antibodies. 2001; 10(2):77-82. Epub 2001/10/24. PubMed PMID: 11673662.
30. Oka Y, Tsuboi A, Oji Y, Kawase I, Sugiyama H. WT1 peptide vaccine for the treatment of cancer. Curr Opin Immunol. 2008; 20(2):211-20. Epub 2008/05/27. doi: 10.1016/j.coi.2008.04.009. PubMed PMID: 18502632.
31. Sugiyama H. Wilms' tumor gene WT1: its oncogenic function and clinical application. Int J Hematol. 2001; 73(2):177-87. Epub 2001/05/25. doi: 10.1007/BF02981935. PubMed PMID: 11372729.
32. Hanada S, Tsuruta T, Haraguchi K, Okamoto M, Sugiyama H, Koido S. Long-term survival of pancreatic cancer patients treated with multimodal therapy combined with WT1-targeted dendritic cell vaccines. Human vaccines & immunotherapeutics. 2019; 15(2):397-406. Epub 2018/09/20. doi: 10.1080/21645515.2018.1524238. PubMed PMID: 30230959; PubMed Central PMCID: PMCPMC6422489.
33. Li Z, Oka Y, Tsuboi A, Fujiki F, Harada Y, Nakajima H, et al. Identification of a WT1 protein-derived peptide, WT1, as a HLA-A 0206-restricted, WT1-specific CTL epitope. Microbiol Immunol. 2008; 52(11):551-8. Epub 2008/12/19. doi: 10.1111/j.1348-0421.2008.00069.x. PubMed PMID: 19090835.
34. Oka Y, Tsuboi A, Nakata J, Nishida S, Hosen N, Kumanogoh A, et al. Wilms' Tumor Gene 1 (WT1) Peptide Vaccine Therapy for Hematological Malignancies: From CTL Epitope Identification to Recent Progress in Clinical Studies Including a Cure-Oriented Strategy. Oncol Res Treat. 2017; 40(11):682-90. Epub 2017/10/19. doi: 10.1159/000481353. PubMed PMID: 29041012.
35. Kan S, Bito T, Shimabuku M, Taguchi J, Ohkusa T, Shimodaira S, et al. Impact of mature dendritic cells pulsed with a novel WT1 helper peptide on the induction of HLAA2 restricted WT1 reactive CD8+ T cells. Int J Oncol. 2020; 57(4):1047-56. Epub 2020/09/19. doi: 10.3892/ijo.2020.5110. PubMed PMID: 32945369.
36. Fakhr E, Modic Z, Cid Arregui A. Recent Developments in Immunotherapy of Cancers Caused by Human Papillomaviruses. Immunology. 2020. Epub 2020/11/19. doi: 10.1111/imm.13285. PubMed PMID: 33205441.
37. Rahman M, Dastmalchi F, Karachi A, Mitchell D. The role of CMV in glioblastoma and implications for immunotherapeutic strategies. Oncoimmunology. 2019; 8(1):e1514921. Epub 2018/12/14. doi: 10.1080/2162402X.2018.1514921. PubMed PMID: 30546954; PubMed Central PMCID: PMCPMC6287786.
38. Batich K A, Reap E A, Archer G E, Sanchez-Perez L, Nair S K, Schmittling R J, et al. Long-term Survival in Glioblastoma with Cytomegalovirus pp65-Targeted Vaccination. Clin Cancer Res. 2017; 23(8):1898-909. Epub 2017/04/16. doi: 10.1158/1078-0432.CCR-16-2057. PubMed PMID: 28411277; PubMed Central PMCID: PMCPMC5559300.
39. Adusumilli P S, Cha E, Cornfeld M, Davis T, Diab A, Dubensky T W, Jr., et al. New Cancer Immunotherapy Agents in Development: a report from an associated program of the 31(st) Annual Meeting of the Society for Immunotherapy of Cancer, 2016. J Immunother Cancer.
2017; 5:50. Epub 2017/06/27. doi: 10.1186/s40425-017-0253-2. PubMed PMID: 28649381; PubMed Central PMCID: PMCPMC5477277.
40. Ott P A, Hu Z, Keskin D B, Shukla S A, Sun J, Bozym D J, et al. An immunogenic personal neoantigen vaccine for patients with melanoma. Nature. 2017; 547(7662):217-21. Epub 2017 Jul. 6. doi: 10.1038/nature22991. PubMed PMID: 28678778; PubMed Central PMCID: PMCPMC5577644.
41. Sahin U, Derhovanessian E, Miller M, Kloke B P, Simon P, Lower M, et al. Personalized RNA mutanome vaccines mobilize poly-specific therapeutic immunity against cancer. Nature. 2017; 547(7662):222-6. Epub 2017/07/06. doi: 10.1038/nature23003. PubMed PMID: 28678784.
42. Li F, Chen C, Ju T, Gao J, Yan J, Wang P, et al. Rapid tumor regression in an Asian lung cancer patient following personalized neo-epitope peptide vaccination. Oncoimmunology. 2016; 5(12):e1238539. Epub 2017/01/27. doi: 10.1080/2162402X.2016.1238539. PubMed PMID: 28123873; PubMed Central PMCID: PMCPMC5214696.
43. Fang Y, Mo F, Shou J, Wang H, Luo K, Zhang S, et al. A Pan-cancer Clinical Study of Personalized Neoantigen Vaccine Monotherapy in Treating Patients with Various Types of Advanced Solid Tumors. Clin Cancer Res. 2020; 26(17):4511-20. Epub 2020/05/23. doi: 10.1158/1078-0432.CCR-19-2881. PubMed PMID: 32439700.
44. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5. Epub 2018/12/21. doi: 10.1038/s41586-018-0810-y. PubMed PMID: 30568303.
45. Keskin D B, Anandappa A J, Sun J, Tirosh I, Mathewson N D, Li S, et al. Neoantigen vaccine generates intratumoral T cell responses in phase Ib glioblastoma trial. Nature. 2019; 565(7738):234-9. Epub 2018/12/21. doi: 10.1038/s41586-018-0792-9. PubMed PMID: 30568305.
46. Fennemann F L, de Vries U M, Figdor C G, Verdoes M. Attacking Tumors From All Sides: Personalized Multiplex Vaccines to Tackle Intratumor Heterogeneity. Frontiers in immunology. 2019; 10:824. Epub 2019/05/02. doi: 10.3389/fimmu.2019.00824. PubMed PMID: 31040852; PubMed Central PMCID: PMCPMC6476980.
47. Rabizadeh S, Garner C, Sanborn J Z, Benz S C, Reddy S, Soon-Shiong P. Comprehensive genomic transcriptomic tumor-normal gene panel analysis for enhanced precision in patients with lung cancer. Oncotarget. 2018; 9(27):19223-32. Epub 2018/05/04. doi: 10.18632/oncotarget.24973. PubMed PMID: 29721196; PubMed Central PMCID: PMCPMC5922390.
48. Yadav M, Jhunjhunwala S, Phung Q T, Lupardus P, Tanguay J, Bumbaca S, et al. Predicting immunogenic tumour mutations by combining mass spectrometry and exome sequencing. Nature. 2014; 515(7528):572-6. Epub 2014/11/28. doi: 10.1038/naturel4001. PubMed PMID: 25428506.
49. Abelin J G, Keskin D B, Sarkizova S, Hartigan C R, Zhang W, Sidney J, et al. Mass Spectrometry Profiling of HLA-Associated Peptidomes in Mono-allelic Cells Enables More Accurate Epitope Prediction. Immunity. 2017; 46(2):315-26. Epub 2017/02/24. doi: 10.1016/j.immuni.2017.02.007. PubMed PMID: 28228285; PubMed Central PMCID: PMCPMC5405381.
50. Hoof I, Peters B, Sidney J, Pedersen L E, Sette A, Lund 0, et al. NetMHCpan, a method for MHC class I binding prediction beyond humans. Immunogenetics. 2009; 61(1):1-13. doi: 10.1007/s00251-008-0341-z [doi].
51. Bailey M H, Tokheim C, Porta-Pardo E, Sengupta S, Bertrand D, Weerasinghe A, et al. Comprehensive Characterization of Cancer Driver Genes and Mutations. Cell. 2018; 173(2):371-85 e18. Epub 2018/04/07. doi: 10.1016/j.cell.2018.02.060. PubMed PMID: 29625053; PubMed Central PMCID: PMCPMC6029450.
52. Chang M T, Asthana S, Gao S P, Lee B H, Chapman J S, Kandoth C, et al. Identifying recurrent mutations in cancer reveals widespread lineage diversity and mutational specificity. Nat Biotechnol. 2016; 34(2):155-63. Epub 2015/12/01. doi: 10.1038/nbt.3391. PubMed PMID: 26619011; PubMed Central PMCID: PMCPMC4744099.
53. Kandoth C, McLellan M D, Vandin F, Ye K, Niu B, Lu C, et al. Mutational landscape and significance across 12 major cancer types. Nature. 2013; 502(7471):333-9. Epub 2013/10/18. doi: 10.1038/naturel2634. PubMed PMID: 24132290; PubMed Central PMCID: PMCPMC3927368.
54. Tamborero D, Gonzalez-Perez A, Perez-Llamas C, Deu-Pons J, Kandoth C, Reimand J, et al. Comprehensive identification of mutational cancer driver genes across 12 tumor types. Scientific reports. 2013; 3:2650. Epub 2013/10/03. doi: 10.1038/srep02650. PubMed PMID: 24084849; PubMed Central PMCID: PMCPMC3788361.
55. Kim H, Kim Y M. Pan-cancer analysis of somatic mutations and transcriptomes reveals common functional gene clusters shared by multiple cancer types. Scientific reports. 2018; 8(1):6041. Epub 2018/04/18. doi: 10.1038/s41598-018-24379-y. PubMed PMID: 29662161; PubMed Central PMCID: PMCPMC5902616.
56. Richters M M, Xia H, Campbell K M, Gillanders W E, Griffith O L, Griffith M. Best practices for bioinformatic characterization of neoantigens for clinical utility. Genome Med. 2019; 11(1):56. Epub 2019/08/30. doi: 10.1186/s13073-019-0666-2. PubMed PMID: 31462330; PubMed Central PMCID: PMCPMC6714459.
57. Cohen C J, Gartner J J, Horovitz-Fried M, Shamalov K, Trebska-McGowan K, Bliskovsky V V, et al. Isolation of neoantigen-specific T cells from tumor and peripheral lymphocytes. J Clin Invest. 2015; 125(10):3981-91. Epub 2015/09/22. doi: 10.1172/JCI82416. PubMed PMID: 26389673; PubMed Central PMCID: PMCPMC4607110.
58. Lauvau G, Soudja S M. Mechanisms of Memory T Cell Activation and Effective Immunity. Adv Exp Med Biol. 2015; 850:73-80. Epub 2015/09/02. doi: 10.1007/978-3-319-15774-0_6. PubMed PMID: 26324347; PubMed Central PMCID: PMCPMC4836952.
59. Zehn D, Lee S Y, Bevan M J. Complete but curtailed T-cell response to very low-affinity antigen. Nature. 2009; 458(7235):211-4. Epub 2009/02/03. doi: 10.1038/nature07657. PubMed PMID: 19182777; PubMed Central PMCID: PMCPMC2735344.
60. Soudja S M, Chandrabos C, Yakob E, Veenstra M, Palliser D, Lauvau G. Memory-T-cell-derived interferon-gamma instructs potent innate cell activation for protective immunity. Immunity. 2014; 40(6):974-88. Epub 2014/06/17. doi: 10.1016/j.immuni.2014.05.005. PubMed PMID: 24931122; PubMed Central PMCID: PMCPMC4105986.
61. Wucherpfennig K W, Allen P M, Celada F, Cohen I R, De Boer R, Garcia K C, et al. Polyspecificity of T cell and B cell receptor recognition. Seminars in immunology. 2007; 19(4):216-24. Epub 2007/04/03. doi: 10.1016/j.smim.2007.02.012. PubMed PMID: 17398114; PubMed Central PMCID: PMC2034306.
62. Benjamin D, Sato, T., Cibulskis, K., Getz, G., Stewart, C., Lichtenstein, L. Calling Somatic SNVs and Indels with Mutect2. bioRXiv. 2019. doi: doi.org/10.1101/861054.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method of treatment of a subject clinically presenting with a tumor comprising:
administering a first round of vaccination, wherein the vaccination comprises an array of one or more peptides, or nucleic acids encoding the peptides, selected from the group consisting of non-mutated peptides derived from tumor associated antigens appropriate to tumors of the type diagnosed and peptides commonly found to be mutated in tumors of the type diagnosed; and
administering a second round of personalized vaccination, wherein the second round of vaccination comprises the following steps:
obtaining a biopsy of the tumor and of normal tissue from the subject, and obtaining sequences for DNA, RNA and proteins in the biopsy;
identifying proteins from the biopsy containing mutated amino acids and the peptide comprising each of the mutated amino acids;
determining T cell exposed motifs which comprise mutated amino acids in each of the proteins;
determining the predicted binding affinity to the subject's MHC alleles of peptides which comprise each of the T cell exposed motifs, or a subset thereof;
generating an array of alternative peptides not present in the tumor, wherein each peptide in the array comprises the amino acids of one of the T cell exposed motifs, and in which the amino acids not within the T cell exposed motif are substituted to change the predicted MHC binding affinity;
selecting a group of one or more selected peptides from the array of alternative peptides which have a desired predicted binding affinity for one or more of the subject's MHC alleles; and synthesizing the group of one or more selected peptides, or nucleic acids encoding the selected peptides; and
administering the selected peptides, or their encoding nucleic acids, as the second round of vaccination.

2. The method of claim 1 further comprising selecting the peptides commonly found to be mutated in tumors so as to position the mutated amino acid in a T cell exposed position and substituting one or more of the amino acids not in a T cell exposed position to provide a desired binding affinity to one or more of the MHC alleles of the subject.

3. The method of claim 1, further comprising:
a. determining the fraction of the DNA in the tumor biopsy which encodes each of the mutated amino acids and the fraction of RNA transcribed from that gene locus and expressing the mutated amino acids; and
b. selecting a sub array of the alternative peptides from the proteins in the biopsy which are present in at least 10% of the DNA in the biopsy and expressed in at least 10% of the RNA transcribed from that gene locus in the biopsy.

4. The method of claim 1, further comprising determining the MHC alleles of the subject prior to the first round of vaccination.

5. The method of claim 1, wherein the first round of vaccination is administered prior to surgical intervention.

6. The method of claim 1, wherein the MHC alleles are MHC type I alleles and the T cell response is a CD8+ response.

7. The method of claim 1, wherein the MHC alleles are MHC type II alleles and the T cell response is a CD4+ response.

8. The method of claim 1, wherein the MHC alleles are a combination of MHC type I alleles and MHC type II alleles.

9. The method of claim 1, wherein the MHC I allele in the first round of vaccination is not A0201 or A2402.

10. The method of claim 1, wherein the peptides, or nucleic acids encoding them, administered in the first round or the second round are 8 to 10 amino acids long.

11. The method of claim 1, wherein the peptides, or nucleic acids encoding them, administered in the first round or the second round are 13-20 amino acids long.

12. The method of claim 1, wherein the peptides, or nucleic acids encoding them, administered in the first round or the second round are 8-35 amino acids long.

13. The method of claim 1, wherein the group of one or more selected peptides, or the nucleic acids encoding them, administered in the second round of vaccination comprises at least 5 unique peptides not present in the proteins sequenced in the tumor.

14. The method of claim 1, wherein the desired predicted binding affinity to the one or more of the subject's MHC alleles of selected peptides in the second round of vaccination exceeds 85% of the binding affinity of all peptides in the tumor protein that comprises the mutated amino acid.

15. The method of claim 1, wherein the desired predicted binding affinity to the one or more of the subject's MHC alleles of selected peptides in the second round exceeds 95% of the binding affinity of all peptides in the tumor protein that comprises the mutated amino acid.

16. The method of claim 1, wherein the desired predicted binding affinity to the one or more of the subject's MHC alleles of selected peptides in the second round is less than 50 nanomolar.

17. The method of claim 1, wherein the desired predicted binding affinity to the one or more of the subject's MHC alleles of selected peptides in the second round is less than 200 nanomolar.

18. The vaccine of claim 1, wherein the first and/or second rounds of vaccination further comprise administering peptides, or the nucleic acids encoding them, which occur naturally in a tumor protein.

19. The method of claim 1, wherein the first and second rounds of vaccination each comprise more than a single application of the array of peptides, or the nucleic acids encoding them.

20. The method of claim 1, wherein each round of vaccination comprises 3 or more applications of the array of peptides, or the nucleic acids encoding them.

21-95. (canceled)


SEQUENCE LISTING (537 sequences)

SEQ ID NO: 1 - protein, 10 aa, Artificial sequence
  synthetic
VAATLFWLLL

SEQ ID NO: 2 - protein, 10 aa, Artificial sequence
  synthetic
VLLWEIFSLG

SEQ ID NO: 3 - protein, 10 aa, Artificial sequence
  synthetic
VIAMFFWLLL

SEQ ID NO: 4 - protein, 10 aa, Artificial sequence
  synthetic
DVWSFGVLLW

SEQ ID NO: 5 - protein, 9 aa, Artificial sequence
  synthetic
VAATLFWLL

SEQ ID NO: 6 - protein, 9 aa, Artificial sequence
  synthetic
AATLFWLLL

SEQ ID NO: 7 - protein, 9 aa, Artificial sequence
  synthetic
VLLWEIFSL

SEQ ID NO: 8 - protein, 9 aa, Artificial sequence
  synthetic
LLWEIFSLG

SEQ ID NO: 9 - protein, 9 aa, Artificial sequence
  synthetic
VIAMFFWLL

SEQ ID NO: 10 - protein, 9 aa, Artificial sequence
  synthetic
IAMFFWLLL

SEQ ID NO: 11 - protein, 9 aa, Artificial sequence
  synthetic
DVWSFGVLL

SEQ ID NO: 12 - protein, 9 aa, Artificial sequence
  synthetic
VWSFGVLLW

SEQ ID NO: 13 - protein, 17 aa, Artificial sequence
  synthetic
ATLFWLLLTLFIRKMKR

SEQ ID NO: 14 - protein, 17 aa, Artificial sequence
  synthetic
KSDVWSYGVLLWEIFSL

SEQ ID NO: 15 - protein, 17 aa, Artificial sequence
  synthetic
GTAVIAMFFWLLLVIIL

SEQ ID NO: 16 - protein, 17 aa, Artificial sequence
  synthetic
DVWSFGVLLWEIFSLGA

SEQ ID NO: 17 - protein, 15 aa, Artificial sequence
  synthetic
ATLFWLLLTLFIRKM

SEQ ID NO: 18 - protein, 15 aa, Artificial sequence
  synthetic
TLFWLLLTLFIRKMK

SEQ ID NO: 19 - protein, 15 aa, Artificial sequence
  synthetic
LFWLLLTLFIRKMKR

SEQ ID NO: 20 - protein, 15 aa, Artificial sequence
  synthetic
KSDVWSYGVLLWEIF

SEQ ID NO: 21 - protein, 15 aa, Artificial sequence
  synthetic
SDVWSYGVLLWEIFS

SEQ ID NO: 22 - protein, 15 aa, Artificial sequence
  synthetic
DVWSYGVLLWEIFSL

SEQ ID NO: 23 - protein, 15 aa, Artificial sequence
  synthetic
GTAVIAMFFWLLLVI

SEQ ID NO: 24 - protein, 15 aa, Artificial sequence
  synthetic
TAVIAMFFWLLLVII

SEQ ID NO: 25 - protein, 15 aa, Artificial sequence
  synthetic
AVIAMFFWLLLVIIL

SEQ ID NO: 26 - protein, 15 aa, Artificial sequence
  synthetic
DVWSFGVLLWEIFSL

SEQ ID NO: 27 - protein, 15 aa, Artificial sequence
  synthetic
VWSFGVLLWEIFSLG

SEQ ID NO: 28 - protein, 15 aa, Artificial sequence
  synthetic
WSFGVLLWEIFSLGA

SEQ ID NO: 29 - protein, 10 aa, Artificial sequence
  synthetic
SLYASSPGGV

SEQ ID NO: 30 - protein, 9 aa, Artificial sequence
  synthetic
YASSPGGVY

SEQ ID NO: 31 - protein, 9 aa, Artificial sequence
  synthetic
SPGGVYATR

SEQ ID NO: 32 - protein, 10 aa, Artificial sequence
  synthetic
GVYATRSSAV

SEQ ID NO: 33 - protein, 10 aa, Artificial sequence
  synthetic
ELQELNDRFA

SEQ ID NO: 34 - protein, 9 aa, Artificial sequence
  synthetic
ELNDRFANY

SEQ ID NO: 35 - protein, 10 aa, Artificial sequence
  synthetic
RFANYIDKVR

SEQ ID NO: 36 - protein, 9 aa, Artificial sequence
  synthetic
NYIDKVRFL

SEQ ID NO: 37 - protein, 10 aa, Artificial sequence
  synthetic
NLQEAEEWYK

SEQ ID NO: 38 - protein, 9 aa, Artificial sequence
  synthetic
EAEEWYKSK

SEQ ID NO: 39 - protein, 10 aa, Artificial sequence
  synthetic
WYKSKFADLS

SEQ ID NO: 40 - protein, 10 aa, Artificial sequence
  synthetic
SKFADLSEAA

SEQ ID NO: 41 - protein, 9 aa, Artificial sequence
  synthetic
SLYASSPGG

SEQ ID NO: 42 - protein, 9 aa, Artificial sequence
  synthetic
LYASSPGGV

SEQ ID NO: 43 - protein, 9 aa, Artificial sequence
  synthetic
YASSPGGVY

SEQ ID NO: 44 - protein, 9 aa, Artificial sequence
  synthetic
SPGGVYATR

SEQ ID NO: 45 - protein, 9 aa, Artificial sequence
  synthetic
GVYATRSSA

SEQ ID NO: 46 - protein, 9 aa, Artificial sequence
  synthetic
VYATRSSAV

SEQ ID NO: 47 - protein, 9 aa, Artificial sequence
  synthetic
ELQELNDRF

SEQ ID NO: 48 - protein, 9 aa, Artificial sequence
  synthetic
LQELNDRFA

SEQ ID NO: 49 - protein, 9 aa, Artificial sequence
  synthetic
ELNDRFANY

SEQ ID NO: 50 - protein, 9 aa, Artificial sequence
  synthetic
RFANYIDKV

SEQ ID NO: 51 - protein, 9 aa, Artificial sequence
  synthetic
FANYIDKVR

SEQ ID NO: 52 - protein, 9 aa, Artificial sequence
  synthetic
NYIDKVRFL

SEQ ID NO: 53 - protein, 9 aa, Artificial sequence
  synthetic
NLQEAEEWY

SEQ ID NO: 54 - protein, 9 aa, Artificial sequence
  synthetic
LQEAEEWYK

SEQ ID NO: 55 - protein, 9 aa, Artificial sequence
  synthetic
EAEEWYKSK

SEQ ID NO: 56 - protein, 9 aa, Artificial sequence
  synthetic
WYKSKFADL

SEQ ID NO: 57 - protein, 9 aa, Artificial sequence
  synthetic
YKSKFADLS

SEQ ID NO: 58 - protein, 9 aa, Artificial sequence
  synthetic
SKFADLSEA

SEQ ID NO: 59 - protein, 9 aa, Artificial sequence
  synthetic
KFADLSEAA

SEQ ID NO: 60 - protein, 17 aa, Artificial sequence
  synthetic
PSTSRSLYASSPGGVYA

SEQ ID NO: 61 - protein, 17 aa, Artificial sequence
  synthetic
SRSLYASSPGGVYATRS

SEQ ID NO: 62 - protein, 16 aa, Artificial sequence
  synthetic
SSAVRLRSSVPGVRLL

SEQ ID NO: 63 - protein, 16 aa, Artificial sequence
  synthetic
AVRLRSSVPGVRLLQD

SEQ ID NO: 64 - protein, 16 aa, Artificial sequence
  synthetic
LNDRFANYIDKVRFLE

SEQ ID NO: 65 - protein, 16 aa, Artificial sequence
  synthetic
DRFANYIDKVRFLEQQ

SEQ ID NO: 66 - protein, 16 aa, Artificial sequence
  synthetic
FANYIDKVRFLEQQNK

SEQ ID NO: 67 - protein, 17 aa, Artificial sequence
  synthetic
NYIDKVRFLEQQNKILL

SEQ ID NO: 68 - protein, 17 aa, Artificial sequence
  synthetic
DKVRFLEQQNKILLAEL

SEQ ID NO: 69 - protein, 16 aa, Artificial sequence
  synthetic
FLEQQNKILLAELEQL

SEQ ID NO: 70 - protein, 16 aa, Artificial sequence
  synthetic
QQNKILLAELEQLKGQ

SEQ ID NO: 71 - protein, 16 aa, Artificial sequence
  synthetic
NKILLAELEQLKGQGK

SEQ ID NO: 72 - protein, 17 aa, Artificial sequence
  synthetic
EAEEWYKSKFADLSEAA

SEQ ID NO: 73 - protein, 15 aa, Artificial sequence
  synthetic
PSTSRSLYASSPGGV

SEQ ID NO: 74 - protein, 15 aa, Artificial sequence
  synthetic
STSRSLYASSPGGVY

SEQ ID NO: 75 - protein, 15 aa, Artificial sequence
  synthetic
TSRSLYASSPGGVYA

SEQ ID NO: 76 - protein, 15 aa, Artificial sequence
  synthetic
SRSLYASSPGGVYAT

SEQ ID NO: 77 - protein, 15 aa, Artificial sequence
  synthetic
RSLYASSPGGVYATR

SEQ ID NO: 78 - protein, 15 aa, Artificial sequence
  synthetic
SLYASSPGGVYATRS

SEQ ID NO: 79 - protein, 15 aa, Artificial sequence
  synthetic
SSAVRLRSSVPGVRL

SEQ ID NO: 80 - protein, 15 aa, Artificial sequence
  synthetic
SAVRLRSSVPGVRLL

SEQ ID NO: 81 - protein, 15 aa, Artificial sequence
  synthetic
AVRLRSSVPGVRLLQ

SEQ ID NO: 82 - protein, 15 aa, Artificial sequence
  synthetic
VRLRSSVPGVRLLQD

SEQ ID NO: 83 - protein, 15 aa, Artificial sequence
  synthetic
LNDRFANYIDKVRFL

SEQ ID NO: 84 - protein, 15 aa, Artificial sequence
  synthetic
NDRFANYIDKVRFLE

SEQ ID NO: 85 - protein, 15 aa, Artificial sequence
  synthetic
DRFANYIDKVRFLEQ

SEQ ID NO: 86 - protein, 15 aa, Artificial sequence
  synthetic
RFANYIDKVRFLEQQ

SEQ ID NO: 87 - protein, 15 aa, Artificial sequence
  synthetic
FANYIDKVRFLEQQN

SEQ ID NO: 88 - protein, 15 aa, Artificial sequence
  synthetic
ANYIDKVRFLEQQNK

SEQ ID NO: 89 - protein, 15 aa, Artificial sequence
  synthetic
NYIDKVRFLEQQNKI

SEQ ID NO: 90 - protein, 15 aa, Artificial sequence
  synthetic
YIDKVRFLEQQNKIL

SEQ ID NO: 91 - protein, 15 aa, Artificial sequence
  synthetic
IDKVRFLEQQNKILL

SEQ ID NO: 92 - protein, 15 aa, Artificial sequence
  synthetic
DKVRFLEQQNKILLA

SEQ ID NO: 93 - protein, 15 aa, Artificial sequence
  synthetic
KVRFLEQQNKILLAE

SEQ ID NO: 94 - protein, 15 aa, Artificial sequence
  synthetic
VRFLEQQNKILLAEL

SEQ ID NO: 95 - protein, 15 aa, Artificial sequence
  synthetic
FLEQQNKILLAELEQ

SEQ ID NO: 96 - protein, 15 aa, Artificial sequence
  synthetic
LEQQNKILLAELEQL

SEQ ID NO: 97 - protein, 15 aa, Artificial sequence
  synthetic
QQNKILLAELEQLKG

SEQ ID NO: 98 - protein, 15 aa, Artificial sequence
  synthetic
QNKILLAELEQLKGQ

SEQ ID NO: 99 - protein, 15 aa, Artificial sequence
  synthetic
NKILLAELEQLKGQG

SEQ ID NO: 100 - protein, 15 aa, Artificial sequence
  synthetic
KILLAELEQLKGQGK

SEQ ID NO: 101 - protein, 15 aa, Artificial sequence
  synthetic
EAEEWYKSKFADLSE

SEQ ID NO: 102 - protein, 15 aa, Artificial sequence
  synthetic
AEEWYKSKFADLSEA

SEQ ID NO: 103 - protein, 15 aa, Artificial sequence
  synthetic
EEWYKSKFADLSEAA

SEQ ID NO: 104 - protein, 10 aa, Artificial sequence
  synthetic
QALLLRTPYS

SEQ ID NO: 105 - protein, 10 aa, Artificial sequence
  synthetic
MFPNAPYLPS

SEQ ID NO: 106 - protein, 9 aa, Artificial sequence
  synthetic
QALLLRTPY

SEQ ID NO: 107 - protein, 9 aa, Artificial sequence
  synthetic
ALLLRTPYS

SEQ ID NO: 108 - protein, 9 aa, Artificial sequence
  synthetic
MFPNAPYLP

SEQ ID NO: 109 - protein, 9 aa, Artificial sequence
  synthetic
FPNAPYLPS

SEQ ID NO: 110 - protein, 17 aa, Artificial sequence
  synthetic
SQALLLRTPYSSDNLYQ

SEQ ID NO: 111 - protein, 17 aa, Artificial sequence
  synthetic
QARMFPNAPYLPSCLES

SEQ ID NO: 112 - skipped

SEQ ID NO: 113 - protein, 15 aa, Artificial sequence
  synthetic
SQALLLRTPYSSDNL

SEQ ID NO: 114 - protein, 15 aa, Artificial sequence
  synthetic
QALLLRTPYSSDNLY

SEQ ID NO: 115 - protein, 15 aa, Artificial sequence
  synthetic
ALLLRTPYSSDNLYQ

SEQ ID NO: 116 - protein, 15 aa, Artificial sequence
  synthetic
QARMFPNAPYLPSCL

SEQ ID NO: 117 - protein, 15 aa, Artificial sequence
  synthetic
ARMFPNAPYLPSCLE

SEQ ID NO: 118 - protein, 15 aa, Artificial sequence
  synthetic
RMFPNAPYLPSCLES

SEQ ID NO: 119-123 - skipped (5 entries)

SEQ ID NO: 124 - protein, 9 aa, Artificial sequence
  synthetic
EGKYSFGDT

SEQ ID NO: 125 - protein, 9 aa, Artificial sequence
  synthetic
GKYSFGDTC

SEQ ID NO: 126 - protein, 9 aa, Artificial sequence
  synthetic
KYSFGDTCV

SEQ ID NO: 127 - protein, 9 aa, Artificial sequence
  synthetic
YSFGDTCVK

SEQ ID NO: 128 - protein, 9 aa, Artificial sequence
  synthetic
SFGDTCVKK

SEQ ID NO: 129 - protein, 9 aa, Artificial sequence
  synthetic
EGKYSFGVT

SEQ ID NO: 130 - protein, 9 aa, Artificial sequence
  synthetic
GKYSFGVTC

SEQ ID NO: 131 - protein, 9 aa, Artificial sequence
  synthetic
KYSFGVTCV

SEQ ID NO: 132 - protein, 9 aa, Artificial sequence
  synthetic
YSFGVTCVK

SEQ ID NO: 133 - protein, 9 aa, Artificial sequence
  synthetic
SFGVTCVKK

SEQ ID NO: 134 - protein, 9 aa, Artificial sequence
  synthetic
EGKYSFGTT

SEQ ID NO: 135 - protein, 9 aa, Artificial sequence
  synthetic
GKYSFGTTC

SEQ ID NO: 136 - protein, 9 aa, Artificial sequence
  synthetic
KYSFGTTCV

SEQ ID NO: 137 - protein, 9 aa, Artificial sequence
  synthetic
YSFGTTCVK

SEQ ID NO: 138 - protein, 9 aa, Artificial sequence
  synthetic
SFGTTCVKK

SEQ ID NO: 139 - protein, 9 aa, Artificial sequence
  synthetic
CVKTCPADV

SEQ ID NO: 140 - protein, 9 aa, Artificial sequence
  synthetic
VKTCPADVM

SEQ ID NO: 141 - protein, 9 aa, Artificial sequence
  synthetic
KTCPADVMG

SEQ ID NO: 142 - protein, 9 aa, Artificial sequence
  synthetic
TCPADVMGE

SEQ ID NO: 143 - protein, 9 aa, Artificial sequence
  synthetic
CPADVMGEN

SEQ ID NO: 144 - protein, 9 aa, Artificial sequence
  synthetic
CVKTCPAVV

SEQ ID NO: 145 - protein, 9 aa, Artificial sequence
  synthetic
VKTCPAVVM

SEQ ID NO: 146 - protein, 9 aa, Artificial sequence
  synthetic
KTCPAVVMG

SEQ ID NO: 147 - protein, 9 aa, Artificial sequence
  synthetic
TCPAVVMGE

SEQ ID NO: 148 - protein, 9 aa, Artificial sequence
  synthetic
CPAVVMGEN

SEQ ID NO: 149 - protein, 9 aa, Artificial sequence
  synthetic
VKITDFGRA

SEQ ID NO: 150 - protein, 9 aa, Artificial sequence
  synthetic
KITDFGRAK

SEQ ID NO: 151 - protein, 9 aa, Artificial sequence
  synthetic
ITDFGRAKL

SEQ ID NO: 152 - protein, 9 aa, Artificial sequence
  synthetic
TDFGRAKLL

SEQ ID NO: 153 - protein, 9 aa, Artificial sequence
  synthetic
DFGRAKLLG

SEQ ID NO: 154 - protein, 5 aa, Artificial sequence
  synthetic
YSFGD

SEQ ID NO: 155 - protein, 5 aa, Artificial sequence
  synthetic
SFGDT

SEQ ID NO: 156 - protein, 5 aa, Artificial sequence
  synthetic
FGDTC

SEQ ID NO: 157 - protein, 5 aa, Artificial sequence
  synthetic
GDTCV

SEQ ID NO: 158 - protein, 5 aa, Artificial sequence
  synthetic
DTCVK

SEQ ID NO: 159 - protein, 5 aa, Artificial sequence
  synthetic
YSFGV

SEQ ID NO: 160 - protein, 5 aa, Artificial sequence
  synthetic
SFGVT

SEQ ID NO: 161 - protein, 5 aa, Artificial sequence
  synthetic
FGVTC

SEQ ID NO: 162 - protein, 5 aa, Artificial sequence
  synthetic
GVTCV

SEQ ID NO: 163 - protein, 5 aa, Artificial sequence
  synthetic
VTCVK

SEQ ID NO: 164 - protein, 5 aa, Artificial sequence
  synthetic
YSFGT

SEQ ID NO: 165 - protein, 5 aa, Artificial sequence
  synthetic
SFGTT

SEQ ID NO: 166 - protein, 5 aa, Artificial sequence
  synthetic
FGTTC

SEQ ID NO: 167 - protein, 5 aa, Artificial sequence
  synthetic
GTTCV

SEQ ID NO: 168 - protein, 5 aa, Artificial sequence
  synthetic
TTCVK

SEQ ID NO: 169 - protein, 5 aa, Artificial sequence
  synthetic
TCPAD

SEQ ID NO: 170 - protein, 5 aa, Artificial sequence
  synthetic
CPADV

SEQ ID NO: 171 - protein, 5 aa, Artificial sequence
  synthetic
PADVM

SEQ ID NO: 172 - protein, 5 aa, Artificial sequence
  synthetic
ADVMG

SEQ ID NO: 173 - protein, 5 aa, Artificial sequence
  synthetic
DVMGE

SEQ ID NO: 174 - protein, 5 aa, Artificial sequence
  synthetic
TCPAV

SEQ ID NO: 175 - protein, 5 aa, Artificial sequence
  synthetic
CPAVV

SEQ ID NO: 176 - protein, 5 aa, Artificial sequence
  synthetic
PAVVM

SEQ ID NO: 177 - protein, 5 aa, Artificial sequence
  synthetic
AVVMG

SEQ ID NO: 178 - protein, 5 aa, Artificial sequence
  synthetic
VVMGE

SEQ ID NO: 179 - protein, 5 aa, Artificial sequence
  synthetic
TDFGR

SEQ ID NO: 180 - protein, 5 aa, Artificial sequence
  synthetic
DFGRA

SEQ ID NO: 181 - protein, 5 aa, Artificial sequence
  synthetic
FGRAK

SEQ ID NO: 182 - protein, 5 aa, Artificial sequence
  synthetic
GRAKL

SEQ ID NO: 183 - protein, 5 aa, Artificial sequence
  synthetic
RAKLL

SEQ ID NO: 184-195 - skipped (12 entries)

SEQ ID NO: 196 - protein, 9 aa, Artificial sequence
  synthetic
KVTYSFGDY

SEQ ID NO: 197 - protein, 9 aa, Artificial sequence
  synthetic
YTYYSFGDI

SEQ ID NO: 198 - protein, 9 aa, Artificial sequence
  synthetic
YKYSFGDTI

SEQ ID NO: 199 - protein, 9 aa, Artificial sequence
  synthetic
EYCSFGDTI

SEQ ID NO: 200 - protein, 9 aa, Artificial sequence
  synthetic
YYVFGDTCI

SEQ ID NO: 201 - protein, 9 aa, Artificial sequence
  synthetic
YYTFGDTCV

SEQ ID NO: 202 - protein, 9 aa, Artificial sequence
  synthetic
YSVGDTCVY

SEQ ID NO: 203 - protein, 9 aa, Artificial sequence
  synthetic
KVVGDTCVY

SEQ ID NO: 204 - protein, 9 aa, Artificial sequence
  synthetic
YVCDTCVKV

SEQ ID NO: 205 - protein, 9 aa, Artificial sequence
  synthetic
IYTDTCVKS

SEQ ID NO: 206 - protein, 9 aa, Artificial sequence
  synthetic
EGVYSFGTE

SEQ ID NO: 207 - protein, 9 aa, Artificial sequence
  synthetic
DVVYSFGTT

SEQ ID NO: 208 - protein, 9 aa, Artificial sequence
  synthetic
EYDSFGTTV

SEQ ID NO: 209 - protein, 9 aa, Artificial sequence
  synthetic
IIGSFGTTS

SEQ ID NO: 210 - protein, 9 aa, Artificial sequence
  synthetic
YYVFGTTCV

SEQ ID NO: 211 - protein, 9 aa, Artificial sequence
  synthetic
YYIFGTTCI

SEQ ID NO: 212 - protein, 9 aa, Artificial sequence
  synthetic
EVVGTTCVV

SEQ ID NO: 213 - protein, 9 aa, Artificial sequence
  synthetic
VTGGTTCVY

SEQ ID NO: 214 - protein, 9 aa, Artificial sequence
  synthetic
ETCTCPADT

SEQ ID NO: 215 - protein, 9 aa, Artificial sequence
  synthetic
TTETCPADY

SEQ ID NO: 216 - protein, 9 aa, Artificial sequence
  synthetic
KYKTCPADV

SEQ ID NO: 217 - protein, 9 aa, Artificial sequence
  synthetic
GYDTCPADV

SEQ ID NO: 218 - protein, 9 aa, Artificial sequence
  synthetic
EGVDVMGEK

SEQ ID NO: 219 - protein, 9 aa, Artificial sequence
  synthetic
DKIDVMGEY

SEQ ID NO: 220 - protein, 9 aa, Artificial sequence
  synthetic
TSGTCPAVY

SEQ ID NO: 221 - protein, 9 aa, Artificial sequence
  synthetic
DSSTCPAVY

SEQ ID NO: 222 - protein, 9 aa, Artificial sequence
  synthetic
KYITCPAVG

SEQ ID NO: 223 - protein, 9 aa, Artificial sequence
  synthetic
EYSTCPAVV

SEQ ID NO: 224 - protein, 9 aa, Artificial sequence
  synthetic
DCCCPAVVY

SEQ ID NO: 225 - protein, 9 aa, Artificial sequence
  synthetic
VVKCPAVVY

SEQ ID NO: 226 - protein, 9 aa, Artificial sequence
  synthetic
TIKCPAVVV

SEQ ID NO: 227 - protein, 9 aa, Artificial sequence
  synthetic
GIECPAVVV

SEQ ID NO: 228 - protein, 9 aa, Artificial sequence
  synthetic
KYKPAVVMI

SEQ ID NO: 229 - protein, 9 aa, Artificial sequence
  synthetic
KYKPAVVMV

SEQ ID NO: 230 - protein, 9 aa, Artificial sequence
  synthetic
EVGVVMGEK

SEQ ID NO: 231 - protein, 9 aa, Artificial sequence
  synthetic
EDVVVMGEY

SEQ ID NO: 232 - protein, 9 aa, Artificial sequence
  synthetic
EVSTDFGRK

SEQ ID NO: 233 - protein, 9 aa, Artificial sequence
  synthetic
GVETDFGRY

SEQ ID NO: 234 - protein, 9 aa, Artificial sequence
  synthetic
TYGTDFGRG

SEQ ID NO: 235 - protein, 9 aa, Artificial sequence
  synthetic
IIKTDFGRI

SEQ ID NO: 236 - protein, 9 aa, Artificial sequence
  synthetic
DTIFGRAKI

SEQ ID NO: 237 - protein, 9 aa, Artificial sequence
  synthetic
EVSFGRAKI

SEQ ID NO: 238 - protein, 9 aa, Artificial sequence
  synthetic
YYTFGRAKI

SEQ ID NO: 239 - protein, 9 aa, Artificial sequence
  synthetic
YYVFGRAKI

SEQ ID NO: 240 - protein, 9 aa, Artificial sequence
  synthetic
SIGGRAKLV

SEQ ID NO: 241 - protein, 9 aa, Artificial sequence
  synthetic
EYYGRAKLI

SEQ ID NO: 242 - protein, 9 aa, Artificial sequence
  synthetic
KYTRAKLLI

SEQ ID NO: 243 - protein, 9 aa, Artificial sequence
  synthetic
GYGRAKLLV

SEQ ID NO: 244 - protein, 9 aa, Artificial sequence
  synthetic
LADKKGNYV

SEQ ID NO: 245 - protein, 9 aa, Artificial sequence
  synthetic
KASEKKGNY

SEQ ID NO: 246 - protein, 9 aa, Artificial sequence
  synthetic
DGDGNYVVS

SEQ ID NO: 247 - protein, 9 aa, Artificial sequence
  synthetic
KLAEKKGNV

SEQ ID NO: 248 - protein, 9 aa, Artificial sequence
  synthetic
QYTKKGNYF

SEQ ID NO: 249 - protein, 9 aa, Artificial sequence
  synthetic
KYTKGNYVW

SEQ ID NO: 250 - protein, 9 aa, Artificial sequence
  synthetic
ESDKGNYVC

SEQ ID NO: 251 - protein, 9 aa, Artificial sequence
  synthetic
APGEEKKGG

SEQ ID NO: 252 - protein, 9 aa, Artificial sequence
  synthetic
PPDKGNYVA

SEQ ID NO: 253 - protein, 9 aa, Artificial sequence
  synthetic
LLREEKKGF

SEQ ID NO: 254 - protein, 9 aa, Artificial sequence
  synthetic
FAMEKKGNY

SEQ ID NO: 255 - protein, 9 aa, Artificial sequence
  synthetic
ECRKGNYVE

SEQ ID NO: 256 - protein, 9 aa, Artificial sequence
  synthetic
PCQKKGNYV

SEQ ID NO: 257 - protein, 9 aa, Artificial sequence
  synthetic
LGDEKKGNF

SEQ ID NO: 258 - protein, 9 aa, Artificial sequence
  synthetic
PASEEKKGF

SEQ ID NO: 259 - protein, 9 aa, Artificial sequence
  synthetic
IRQKGNYVS

SEQ ID NO: 260 - protein, 9 aa, Artificial sequence
  synthetic
LWSEKKGNG

SEQ ID NO: 261 - protein, 9 aa, Artificial sequence
  synthetic
TKSKKGNYR

SEQ ID NO: 262 - protein, 9 aa, Artificial sequence
  synthetic
IRRGNYVVS

SEQ ID NO: 263 - protein, 9 aa, Artificial sequence
  synthetic
LKEEEKKGD

SEQ ID NO: 264 - protein, 9 aa, Artificial sequence
  synthetic
RAEGNYVVR

SEQ ID NO: 265 - protein, 9 aa, Artificial sequence
  synthetic
MGEKKGNYD

SEQ ID NO: 266 - protein, 9 aa, Artificial sequence
  synthetic
TADEKKGNF

SEQ ID NO: 267 - protein, 9 aa, Artificial sequence
  synthetic
RLKEKKGNV

SEQ ID NO: 268 - protein, 9 aa, Artificial sequence
  synthetic
QLPKKGNYI

SEQ ID NO: 269 - protein, 9 aa, Artificial sequence
  synthetic
TKGKGNYVI

SEQ ID NO: 270 - protein, 9 aa, Artificial sequence
  synthetic
EVSKGNYVA

SEQ ID NO: 271 - protein, 9 aa, Artificial sequence
  synthetic
NVRKGNYVA

SEQ ID NO: 272 - protein, 9 aa, Artificial sequence
  synthetic
RTQEEKKGI

SEQ ID NO: 273 - protein, 9 aa, Artificial sequence
  synthetic
QSCEKKGNW

SEQ ID NO: 274 - protein, 9 aa, Artificial sequence
  synthetic
FPMEEKKGR

SEQ ID NO: 275 - protein, 9 aa, Artificial sequence
  synthetic
SEEKKGNYQ

SEQ ID NO: 276 - protein, 9 aa, Artificial sequence
  synthetic
LELKGNYVP

SEQ ID NO: 277 - protein, 9 aa, Artificial sequence
  synthetic
EGPEEKKGY

SEQ ID NO: 278 - protein, 9 aa, Artificial sequence
  synthetic
ISKEKKGNF

SEQ ID NO: 279 - protein, 9 aa, Artificial sequence
  synthetic
EHMKGNYVG

SEQ ID NO: 280 - protein, 9 aa, Artificial sequence
  synthetic
RELEKKGNA

SEQ ID NO: 281 - protein, 9 aa, Artificial sequence
  synthetic
AEHGNYVVT

SEQ ID NO: 282 - protein, 9 aa, Artificial sequence
  synthetic
TRVKKGNYS

SEQ ID NO: 283 - protein, 9 aa, Artificial sequence
  synthetic
WKEEEKKGR

SEQ ID NO: 284 - protein, 5 aa, Artificial sequence
  synthetic
EEKKG

SEQ ID NO: 285 - protein, 5 aa, Artificial sequence
  synthetic
EKKGN

SEQ ID NO: 286 - protein, 5 aa, Artificial sequence
  synthetic
KKGNY

SEQ ID NO: 287 - protein, 5 aa, Artificial sequence
  synthetic
KGNYV

SEQ ID NO: 288 - protein, 5 aa, Artificial sequence
  synthetic
GNYVV

SEQ ID NO: 289 - protein, 9 aa, Artificial sequence
  synthetic
VKPIIIGHH

SEQ ID NO: 290 - protein, 9 aa, Artificial sequence
  synthetic
KPIIIGHHA

SEQ ID NO: 291 - protein, 9 aa, Artificial sequence
  synthetic
PIIIGHHAY

SEQ ID NO: 292 - protein, 9 aa, Artificial sequence
  synthetic
IIIGHHAYG

SEQ ID NO: 293 - protein, 9 aa, Artificial sequence
  synthetic
IIGHHAYGD

SEQ ID NO: 294 - protein, 5 aa, Artificial sequence
  synthetic
IIIGH

SEQ ID NO: 295 - protein, 5 aa, Artificial sequence
  synthetic
IIGHH

SEQ ID NO: 296 - protein, 5 aa, Artificial sequence
  synthetic
IGHHA

SEQ ID NO: 297 - protein, 5 aa, Artificial sequence
  synthetic
GHHAY

SEQ ID NO: 298 - protein, 5 aa, Artificial sequence
  synthetic
HHAYG

SEQ ID NO: 299 - protein, 9 aa, Artificial sequence
  synthetic
HGDIIIGHK

SEQ ID NO: 300 - protein, 9 aa, Artificial sequence
  synthetic
KADIIIGHK

SEQ ID NO: 301 - protein, 9 aa, Artificial sequence
  synthetic
EWKIIIGHI

SEQ ID NO: 302 - protein, 9 aa, Artificial sequence
  synthetic
KYTIIIGHL

SEQ ID NO: 303 - protein, 9 aa, Artificial sequence
  synthetic
NVEIIIGHR

SEQ ID NO: 304 - protein, 9 aa, Artificial sequence
  synthetic
YNAIIIGHK

SEQ ID NO: 305 - protein, 9 aa, Artificial sequence
  synthetic
ARPIIIGHN

SEQ ID NO: 306 - protein, 9 aa, Artificial sequence
  synthetic
KKMIIIGHA

SEQ ID NO: 307 - protein, 9 aa, Artificial sequence
  synthetic
QSDIIGHHR

SEQ ID NO: 308 - protein, 9 aa, Artificial sequence
  synthetic
NGDIIGHHS

SEQ ID NO: 309 - protein, 9 aa, Artificial sequence
  synthetic
NGQIIGHHI

SEQ ID NO: 310 - protein, 9 aa, Artificial sequence
  synthetic
RGNIIGHHR

SEQ ID NO: 311 - protein, 9 aa, Artificial sequence
  synthetic
YSDIGHHAK

SEQ ID NO: 312 - protein, 9 aa, Artificial sequence
  synthetic
KGDIGHHAL

SEQ ID NO: 313 - protein, 9 aa, Artificial sequence
  synthetic
EYLIGHHAF

SEQ ID NO: 314 - protein, 9 aa, Artificial sequence
  synthetic
DWRIGHHAF

SEQ ID NO: 315 - protein, 9 aa, Artificial sequence
  synthetic
EVLIGHHAP

SEQ ID NO: 316 - protein, 9 aa, Artificial sequence
  synthetic
QPFIGHHAY

SEQ ID NO: 317 - protein, 9 aa, Artificial sequence
  synthetic
EINIGHHAK

SEQ ID NO: 318 - protein, 9 aa, Artificial sequence
  synthetic
IADIGHHAK

SEQ ID NO: 319 - protein, 9 aa, Artificial sequence
  synthetic
QRKIGHHAT

SEQ ID NO: 320 - protein, 9 aa, Artificial sequence
  synthetic
SRIIGHHAS

SEQ ID NO: 321 - protein, 9 aa, Artificial sequence
  synthetic
AELIGHHAN

SEQ ID NO: 322 - protein, 9 aa, Artificial sequence
  synthetic
GEYIGHHAE

SEQ ID NO: 323 - protein, 9 aa, Artificial sequence
  synthetic
QAKIGHHAY

SEQ ID NO: 324 - protein, 9 aa, Artificial sequence
  synthetic
EQSIGHHAW

SEQ ID NO: 325 - protein, 9 aa, Artificial sequence
  synthetic
LDDGHHAYA

SEQ ID NO: 326 - protein, 9 aa, Artificial sequence
  synthetic
RADGHHAYA

SEQ ID NO: 327 - protein, 9 aa, Artificial sequence
  synthetic
AVTGHHAYP

SEQ ID NO: 328 - protein, 9 aa, Artificial sequence
  synthetic
NPGGHHAYF

SEQ ID NO: 329 - protein, 9 aa, Artificial sequence
  synthetic
NIRGHHAYV

SEQ ID NO: 330 - protein, 9 aa, Artificial sequence
  synthetic
VPRGHHAYL

SEQ ID NO: 331 - protein, 9 aa, Artificial sequence
  synthetic
FTDHHAYGT

SEQ ID NO: 332 - protein, 9 aa, Artificial sequence
  synthetic
DLQHHAYGY

SEQ ID NO: 333 - protein, 9 aa, Artificial sequence
  synthetic
KRAHHAYGW

SEQ ID NO: 334 - protein, 9 aa, Artificial sequence
  synthetic
RRTHHAYGW

SEQ ID NO: 335 - protein, 9 aa, Artificial sequence
  synthetic
QGMHHAYGR

SEQ ID NO: 336 - protein, 9 aa, Artificial sequence
  synthetic
DGIHHAYGR

SEQ ID NO: 337 - protein, 9 aa, Artificial sequence
  synthetic
EEGHHAYGK

SEQ ID NO: 338 - protein, 9 aa, Artificial sequence
  synthetic
FEIHHAYGT

SEQ ID NO: 339 - protein, 15 aa, Artificial sequence
  synthetic
SGWVKPIIIGHHAYG

SEQ ID NO: 340 - protein, 15 aa, Artificial sequence
  synthetic
GWVKPIIIGHHAYGD

SEQ ID NO: 341 - protein, 15 aa, Artificial sequence
  synthetic
VKPIIIGHHAYGDQY

SEQ ID NO: 342 - protein, 15 aa, Artificial sequence
  synthetic
PIIIGHHAYGDQYRA

SEQ ID NO: 343 - protein, 15 aa, Artificial sequence
  synthetic
IIIGHHAYGDQYRAT

SEQ ID NO: 344 - protein, 5 aa, Artificial sequence
  synthetic
KPIGH

SEQ ID NO: 345 - protein, 5 aa, Artificial sequence
  synthetic
PIIHH

SEQ ID NO: 346 - protein, 5 aa, Artificial sequence
  synthetic
IIHAY

SEQ ID NO: 347 - protein, 5 aa, Artificial sequence
  synthetic
GHAGD

SEQ ID NO: 348 - protein, 5 aa, Artificial sequence
  synthetic
HHYDQ

SEQ ID NO: 349 - protein, 15 aa, Artificial sequence
  synthetic
PMKFKPIIIGHPKGN

SEQ ID NO: 350 - protein, 15 aa, Artificial sequence
  synthetic
LLLRKPIIIGHAKRS

SEQ ID NO: 351 - protein, 15 aa, Artificial sequence
  synthetic
IVWFKPIIIGHKRDA

SEQ ID NO: 352 - protein, 15 aa, Artificial sequence
  synthetic
WVLWKPIIIGHKEQA

SEQ ID NO: 353 - protein, 15 aa, Artificial sequence
  synthetic
YQPWKPIIIGHGEKH

SEQ ID NO: 354 - protein, 15 aa, Artificial sequence
  synthetic
SMIPKPIIIGHNKWT

SEQ ID NO: 355 - protein, 15 aa, Artificial sequence
  synthetic
NLTIKPIIIGHLTRN

SEQ ID NO: 356 - protein, 15 aa, Artificial sequence
  synthetic
PFRIKPIIIGHRIQT

SEQ ID NO: 357 - protein, 15 aa, Artificial sequence
  synthetic
TDLMPIIIGHHDKIH

SEQ ID NO: 358 - protein, 15 aa, Artificial sequence
  synthetic
KGIYPIIIGHHHGQL

SEQ ID NO: 359 - protein, 15 aa, Artificial sequence
  synthetic
LNIFPIIIGHHKNNK

SEQ ID NO: 360 - protein, 15 aa, Artificial sequence
  synthetic
YIKQPIIIGHHAKTF

SEQ ID NO: 361 - protein, 15 aa, Artificial sequence
  synthetic
ATFQPIIIGHHPKQP

SEQ ID NO: 362 - protein, 15 aa, Artificial sequence
  synthetic
GWIEPIIIGHHQQNM

SEQ ID NO: 363 - protein, 15 aa, Artificial sequence
  synthetic
YHILPIIIGHHSQNN

SEQ ID NO: 364 - protein, 15 aa, Artificial sequence
  synthetic
GGIIPIIIGHHEDRS

SEQ ID NO: 365 - protein, 15 aa, Artificial sequence
  synthetic
PKVIIIGHHAYAKGK

SEQ ID NO: 366 - protein, 15 aa, Artificial sequence
  synthetic
QYQIIIGHHAYEQGA

SEQ ID NO: 367 - protein, 15 aa, Artificial sequence
  synthetic
GPLIIIGHHAYKEQF

SEQ ID NO: 368 - protein, 15 aa, Artificial sequence
  synthetic
HFLMIIGHHAYEDNA

SEQ ID NO: 369 - protein, 15 aa, Artificial sequence
  synthetic
NIWAIIGHHAYWDEG

SEQ ID NO: 370 - protein, 15 aa, Artificial sequence
  synthetic
KNYMIIGHHAYYEKS

SEQ ID NO: 371 - protein, 15 aa, Artificial sequence
  synthetic
KQTRIIGHHAYILLQ

SEQ ID NO: 372 - protein, 15 aa, Artificial sequence
  synthetic
KPDIIIGHHAYKDFE

SEQ ID NO: 373 - protein, 15 aa, Artificial sequence
  synthetic
PWIFGHHAYGDKSGL

SEQ ID NO: 374 - protein, 15 aa, Artificial sequence
  synthetic
FLILGHHAYGDNKSM

SEQ ID NO: 375 - protein, 15 aa, Artificial sequence
  synthetic
LMFWGHHAYGDEIYF

SEQ ID NO: 376 - protein, 15 aa, Artificial sequence
  synthetic
IFFWGHHAYGDIKKD

SEQ ID NO: 377 - protein, 15 aa, Artificial sequence
  synthetic
AKFWGHHAYGDLSRP

SEQ ID NO: 378 - protein, 15 aa, Artificial sequence
  synthetic
KFIFGHHAYGDYDKK

SEQ ID NO: 379 - protein, 15 aa, Artificial sequence
  synthetic
TAYVGHHAYGDWYYI

SEQ ID NO: 380 - protein, 15 aa, Artificial sequence
  synthetic
ELIFGHHAYGDWFLI

SEQ ID NO: 381 - protein, 15 aa, Artificial sequence
  synthetic
KRVFHHAYGDQAITL

SEQ ID NO: 382 - protein, 15 aa, Artificial sequence
  synthetic
GRLFHHAYGDQLLDL

SEQ ID NO: 383 - protein, 15 aa, Artificial sequence
  synthetic
MFFYHHAYGDQIKDN

SEQ ID NO: 384 - protein, 15 aa, Artificial sequence
  synthetic
LYYFHHAYGDQQATD

SEQ ID NO: 385 - protein, 15 aa, Artificial sequence
  synthetic
TVLRHHAYGDQWYKS

SEQ ID NO: 386 - protein, 15 aa, Artificial sequence
  synthetic
IKMLHHAYGDQLDDT

SEQ ID NO: 387 - protein, 9 aa, Artificial sequence
  synthetic
LATKAARMS

SEQ ID NO: 388 - protein, 9 aa, Artificial sequence
  synthetic
ATKAARMSA

SEQ ID NO: 389 - protein, 9 aa, Artificial sequence
  synthetic
TKAARMSAP

SEQ ID NO: 390 - protein, 9 aa, Artificial sequence
  synthetic
KAARMSAPS

SEQ ID NO: 391 - protein, 9 aa, Artificial sequence
  synthetic
AARMSAPST

SEQ ID NO: 392 - protein, 5 aa, Artificial sequence
  synthetic
KAARM

SEQ ID NO: 393 - protein, 5 aa, Artificial sequence
  synthetic
AARMS

SEQ ID NO: 394 - protein, 5 aa, Artificial sequence
  synthetic
ARMSA

SEQ ID NO: 395 - protein, 5 aa, Artificial sequence
  synthetic
RMSAP

SEQ ID NO: 396 - protein, 5 aa, Artificial sequence
  synthetic
MSAPS

SEQ ID NO: 397 - protein, 9 aa, Artificial sequence
  synthetic
KATKAARMY

SEQ ID NO: 398 - protein, 9 aa, Artificial sequence
  synthetic
FAQKAARMR

SEQ ID NO: 399 - protein, 9 aa, Artificial sequence
  synthetic
TQRKAARMY

SEQ ID NO: 400 - protein, 9 aa, Artificial sequence
  synthetic
LKKKAARMY

SEQ ID NO: 401 - protein, 9 aa, Artificial sequence
  synthetic
PVEAARMSR

SEQ ID NO: 402 - protein, 9 aa, Artificial sequence
  synthetic
RPDAARMSR

SEQ ID NO: 403 - protein, 9 aa, Artificial sequence
  synthetic
KAKAARMSL

SEQ ID NO: 404 - protein, 9 aa, Artificial sequence
  synthetic
RGKAARMSA

SEQ ID NO: 405 - protein, 9 aa, Artificial sequence
  synthetic
EQEAARMSY

SEQ ID NO: 406 - protein, 9 aa, Artificial sequence
  synthetic
GTRAARMSY

SEQ ID NO: 407 - protein, 9 aa, Artificial sequence
  synthetic
SSEARMSAR

SEQ ID NO: 408 - protein, 9 aa, Artificial sequence
  synthetic
EPDARMSAQ

SEQ ID NO: 409 - protein, 9 aa, Artificial sequence
  synthetic
KSRARMSAL

SEQ ID NO: 410 - protein, 9 aa, Artificial sequence
  synthetic
RTRARMSAV

SEQ ID NO: 411 - protein, 9 aa, Artificial sequence
  synthetic
QRDARMSAL

SEQ ID NO: 412 - protein, 9 aa, Artificial sequence
  synthetic
KPGARMSAA

SEQ ID NO: 413 - protein, 9 aa, Artificial sequence
  synthetic
RLDRMSAPQ

SEQ ID NO: 414 - protein, 9 aa, Artificial sequence
  synthetic
FQERMSAPR

SEQ ID NO: 415 - protein, 9 aa, Artificial sequence
  synthetic
KLTRMSAPT

SEQ ID NO: 416 - protein, 9 aa, Artificial sequence
  synthetic
RQIRMSAPA

SEQ ID NO: 417 - protein, 9 aa, Artificial sequence
  synthetic
NLIRMSAPR

SEQ ID NO: 418 - protein, 9 aa, Artificial sequence
  synthetic
RQYRMSAPK

SEQ ID NO: 419 - protein, 9 aa, Artificial sequence
  synthetic
RPRRMSAPD

SEQ ID NO: 420 - protein, 9 aa, Artificial sequence
  synthetic
KPSRMSAPG

SEQ ID NO: 421 - protein, 9 aa, Artificial sequence
  synthetic
QTRRMSAPA

SEQ ID NO: 422 - protein, 9 aa, Artificial sequence
  synthetic
RDRRMSAPI

SEQ ID NO: 423 - protein, 9 aa, Artificial sequence
  synthetic
DDARMSAPY

SEQ ID NO: 424 - protein, 9 aa, Artificial sequence
  synthetic
KERRMSAPF

SEQ ID NO: 425 - protein, 9 aa, Artificial sequence
  synthetic
LEEMSAPSP

SEQ ID NO: 426 - protein, 9 aa, Artificial sequence
  synthetic
KAAMSAPSY

SEQ ID NO: 427 - protein, 9 aa, Artificial sequence
  synthetic
DTGMSAPSR

SEQ ID NO: 428 - protein, 9 aa, Artificial sequence
  synthetic
ESTMSAPSR

SEQ ID NO: 429 - protein, 9 aa, Artificial sequence
  synthetic
RPSMSAPSS

SEQ ID NO: 430 - protein, 9 aa, Artificial sequence
  synthetic
DPKMSAPSA

SEQ ID NO: 431 - protein, 9 aa, Artificial sequence
  synthetic
KTKMSAPSM

SEQ ID NO: 432 - protein, 9 aa, Artificial sequence
  synthetic
KAQMSAPSY

SEQ ID NO: 433 - protein, 9 aa, Artificial sequence
  synthetic
ASVMSAPSF

SEQ ID NO: 434 - protein, 9 aa, Artificial sequence
  synthetic
RAQMSAPSY

SEQ ID NO: 435 - protein, 15 aa, Artificial sequence
  synthetic
RKQLATKAARMSAPS

SEQ ID NO: 436 - protein, 15 aa, Artificial sequence
  synthetic
KQLATKAARMSAPST

SEQ ID NO: 437 - protein, 15 aa, Artificial sequence
  synthetic
LATKAARMSAPSTGG

SEQ ID NO: 438 - protein, 15 aa, Artificial sequence
  synthetic
TKAARMSAPSTGGVK

SEQ ID NO: 439 - protein, 15 aa, Artificial sequence
  synthetic
KAARMSAPSTGGVKK

SEQ ID NO: 440 - protein, 15 aa, Artificial sequence
  synthetic
AARMSAPSTGGVKKP

SEQ ID NO: 441 - protein, 5 aa, Artificial sequence
  synthetic
ATARM

SEQ ID NO: 442 - protein, 5 aa, Artificial sequence
  synthetic
TKAMS

SEQ ID NO: 443 - protein, 5 aa, Artificial sequence
  synthetic
AAMAP

SEQ ID NO: 444 - protein, 5 aa, Artificial sequence
  synthetic
RMAST

SEQ ID NO: 445 - protein, 5 aa, Artificial sequence
  synthetic
MSPTG

SEQ ID NO: 446 - protein, 5 aa, Artificial sequence
  synthetic
SASGG

SEQ ID NO: 447 - protein, 15 aa, Artificial sequence
  synthetic
QLLIATKAARMSVDV

SEQ ID NO: 448 - protein, 15 aa, Artificial sequence
  synthetic
TMIIATKAARMFARK

SEQ ID NO: 449 - protein, 15 aa, Artificial sequence
  synthetic
DRLRATKAARMGEAA

SEQ ID NO: 450 - protein, 15 aa, Artificial sequence
  synthetic
GRITATKAARMEMQI

SEQ ID NO: 451 - protein, 15 aa, Artificial sequence
  synthetic
KFLRTKAARMSTYDA

SEQ ID NO: 452 - protein, 15 aa, Artificial sequence
  synthetic
PKFRTKAARMSTFPR

SEQ ID NO: 453 - protein, 15 aa, Artificial sequence
  synthetic
AKKYAARMSAPTRKG

SEQ ID NO: 454 - protein, 15 aa, Artificial sequence
  synthetic
KRATAARMSAPCATH

SEQ ID NO: 455 - protein, 15 aa, Artificial sequence
  synthetic
RARLRMSAPSTIAQG

SEQ ID NO: 456 - protein, 15 aa, Artificial sequence
  synthetic
TRAFRMSAPSTLADQ

SEQ ID NO: 457 - protein, 15 aa, Artificial sequence
  synthetic
EAILMSAPSTGIASE

SEQ ID NO: 458 - protein, 15 aa, Artificial sequence
  synthetic
RPRIMSAPSTGLETA

SEQ ID NO: 459 - protein, 15 aa, Artificial sequence
  synthetic
AGRRMSAPSTGQGYC

SEQ ID NO: 460 - protein, 15 aa, Artificial sequence
  synthetic
KRMRMSAPSTGGDLI

SEQ ID NO: 461 - protein, 15 aa, Artificial sequence
  synthetic
ALFFSAPSTGGVRQQ

SEQ ID NO: 462 - protein, 15 aa, Artificial sequence
  synthetic
FIFLSAPSTGGAKLQ

SEQ ID NO: 463 - protein, 9 aa, Artificial sequence
  synthetic
WSGSHQFEQ

SEQ ID NO: 464 - protein, 9 aa, Artificial sequence
  synthetic
SGSHQFEQL

SEQ ID NO: 465 - protein, 9 aa, Artificial sequence
  synthetic
GSHQFEQLS

SEQ ID NO: 466 - protein, 9 aa, Artificial sequence
  synthetic
SHQFEQLSG

SEQ ID NO: 467 - protein, 9 aa, Artificial sequence
  synthetic
HQFEQLSGS

SEQ ID NO: 468 - protein, 5 aa, Artificial sequence
  synthetic
SHQFE

SEQ ID NO: 469 - protein, 5 aa, Artificial sequence
  synthetic
HQFEQ

SEQ ID NO: 470 - protein, 5 aa, Artificial sequence
  synthetic
QFEQL

SEQ ID NO: 471 - protein, 5 aa, Artificial sequence
  synthetic
FEQLS

SEQ ID NO: 472 - protein, 5 aa, Artificial sequence
  synthetic
EQLSG

SEQ ID NO: 473 - protein, 9 aa, Artificial sequence
  synthetic
KLVSHQFEY

SEQ ID NO: 474 - protein, 9 aa, Artificial sequence
  synthetic
SIDSHQFER

SEQ ID NO: 475 - protein, 9 aa, Artificial sequence
  synthetic
AAASHQFEF

SEQ ID NO: 476 - protein, 9 aa, Artificial sequence
  synthetic
SSKSHQFEF

SEQ ID NO: 477 - protein, 9 aa, Artificial sequence
  synthetic
GAISHQFER

SEQ ID NO: 478 - protein, 9 aa, Artificial sequence
  synthetic
HSPSHQFEL

SEQ ID NO: 479 - protein, 9 aa, Artificial sequence
  synthetic
KLYHQFEQS

SEQ ID NO: 480 - protein, 9 aa, Artificial sequence
  synthetic
AMDHQFEQA

SEQ ID NO: 481 - protein, 9 aa, Artificial sequence
  synthetic
FPQHQFEQP

SEQ ID NO: 482 - protein, 9 aa, Artificial sequence
  synthetic
IPLHQFEQY

SEQ ID NO: 483 - protein, 9 aa, Artificial sequence
  synthetic
SSTHQFEQF

SEQ ID NO: 484 - protein, 9 aa, Artificial sequence
  synthetic
KVPHQFEQY

SEQ ID NO: 485 - protein, 9 aa, Artificial sequence
  synthetic
LSSHQFEQF

SEQ ID NO: 486 - protein, 9 aa, Artificial sequence
  synthetic
DSFHQFEQL

SEQ ID NO: 487 - protein, 9 aa, Artificial sequence
  synthetic
PIHHQFEQC

SEQ ID NO: 488 - protein, 9 aa, Artificial sequence
  synthetic
ELAHQFEQG

SEQ ID NO: 489 - protein, 9 aa, Artificial sequence
  synthetic
LALHQFEQM

SEQ ID NO: 490 - protein, 9 aa, Artificial sequence
  synthetic
GEEHQFEQW

SEQ ID NO: 491 - protein, 9 aa, Artificial sequence
  synthetic
SAKQFEQLR

SEQ ID NO: 492 - protein, 9 aa, Artificial sequence
  synthetic
GEAQFEQLK

SEQ ID NO: 493 - protein, 9 aa, Artificial sequence
  synthetic
EVKQFEQLK

SEQ ID NO: 494 - protein, 9 aa, Artificial sequence
  synthetic
RVDQFEQLG

SEQ ID NO: 495 - protein, 9 aa, Artificial sequence
  synthetic
DFKFEQLSA

SEQ ID NO: 496 - protein, 9 aa, Artificial sequence
  synthetic
GGRFEQLSN

SEQ ID NO: 497 - protein, 9 aa, Artificial sequence
  synthetic
YKLFEQLSI

SEQ ID NO: 498 - protein, 9 aa, Artificial sequence
  synthetic
YLGFEQLSA

SEQ ID NO: 499 - protein, 9 aa, Artificial sequence
  synthetic
IMHEQLSGS

SEQ ID NO: 500 - protein, 9 aa, Artificial sequence
  synthetic
KIEEQLSGL

SEQ ID NO: 501 - protein, 9 aa, Artificial sequence
  synthetic
DEAEQLSGF

SEQ ID NO: 502 - protein, 9 aa, Artificial sequence
  synthetic
FYLEQLSGF

SEQ ID NO: 503 - protein, 9 aa, Artificial sequence
  synthetic
TTKEQLSGS

SEQ ID NO: 504 - protein, 9 aa, Artificial sequence
  synthetic
RSQEQLSGA

SEQ ID NO: 505 - protein, 9 aa, Artificial sequence
  synthetic
EAVEQLSGS

SEQ ID NO: 506 - protein, 9 aa, Artificial sequence
  synthetic
NQREQLSGY

SEQ ID NO: 507 - protein, 9 aa, Artificial sequence
  synthetic
SLGEQLSGA

SEQ ID NO: 508 - protein, 9 aa, Artificial sequence
  synthetic
MNVEQLSGL

SEQ ID NO: 509 - protein, 15 aa, Artificial sequence
  synthetic
WSGSHQFEQLSGSIL

SEQ ID NO: 510 - protein, 15 aa, Artificial sequence
  synthetic
GSHQFEQLSGSILWM

SEQ ID NO: 511 - protein, 15 aa, Artificial sequence
  synthetic
SHQFEQLSGSILWMA

SEQ ID NO: 512 - protein, 5 aa, Artificial sequence
  synthetic
HQELS

SEQ ID NO: 513 - protein, 5 aa, Artificial sequence
  synthetic
FELGS

SEQ ID NO: 514 - protein, 5 aa, Artificial sequence
  synthetic
EQSSI

SEQ ID NO: 515 - protein, 15 aa, Artificial sequence
  synthetic
IHKFHQFEQLSPPSQ

SEQ ID NO: 516 - protein, 15 aa, Artificial sequence
  synthetic
PHFIHQFEQLSGFAM

SEQ ID NO: 517 - protein, 15 aa, Artificial sequence
  synthetic
YRYYHQFEQLSMPQV

SEQ ID NO: 518 - protein, 15 aa, Artificial sequence
  synthetic
HMMFHQFEQLSKLQL

SEQ ID NO: 519 - protein, 15 aa, Artificial sequence
  synthetic
WLPYHQFEQLSSPIP

SEQ ID NO: 520 - protein, 15 aa, Artificial sequence
  synthetic
FFECHQFEQLSWLSV

SEQ ID NO: 521 - protein, 15 aa, Artificial sequence
  synthetic
SSWPFEQLSGSTGDN

SEQ ID NO: 522 - protein, 15 aa, Artificial sequence
  synthetic
GPGMFEQLSGSTELF

SEQ ID NO: 523 - protein, 15 aa, Artificial sequence
  synthetic
LEVFFEQLSGSSLAN

SEQ ID NO: 524 - protein, 15 aa, Artificial sequence
  synthetic
RRILFEQLSGSCVGL

SEQ ID NO: 525 - protein, 15 aa, Artificial sequence
  synthetic
WYDIFEQLSGSNAPS

SEQ ID NO: 526 - protein, 15 aa, Artificial sequence
  synthetic
LVFIFEQLSGSNWRA

SEQ ID NO: 527 - protein, 15 aa, Artificial sequence
  synthetic
RGAYFEQLSGSVMFS

SEQ ID NO: 528 - protein, 15 aa, Artificial sequence
  synthetic
FYFYFEQLSGSLDSG

SEQ ID NO: 529 - protein, 15 aa, Artificial sequence
  synthetic
PPLFEQLSGSIPICM

SEQ ID NO: 530 - protein, 15 aa, Artificial sequence
  synthetic
YPRFEQLSGSIKIIG

SEQ ID NO: 531 - protein, 15 aa, Artificial sequence
  synthetic
PTGQEQLSGSIIVIF

SEQ ID NO: 532 - protein, 15 aa, Artificial sequence
  synthetic
PKVLEQLSGSIFVGF

SEQ ID NO: 533 - protein, 15 aa, Artificial sequence
  synthetic
IFHFEQLSGSIFILI

SEQ ID NO: 534 - protein, 15 aa, Artificial sequence
  synthetic
RFRFEQLSGSILSLT

SEQ ID NO: 535 - protein, 15 aa, Artificial sequence
  synthetic
SGSILWMAPEVIRMQ

SEQ ID NO: 536 - protein, 15 aa, Artificial sequence
  synthetic
GSILWMAPEVIRMQD

SEQ ID NO: 537 - protein, 15 aa, Artificial sequence
  synthetic
SILWMAPEVIRMQDK